(19) United States
(12) Patent Application Publication
(10) Pub. No.: US 2013/0097725 A1
(43) Pub. Date: Apr. 18, 2013

(54) SOYBEAN NODULATION FACTOR RECEPTOR PROTEINS, ENCODING NUCLEIC ACIDS AND USES THEREFOR

(71) Applicants: Arief Indrasumunar, Jawa Barat, ID (US); Attila Kereszt, Szeged (HU); Michael Peter Gresshoff, Queensland (AU)

(72) Inventors: Arief Indrasumunar, Jawa Barat, ID (US); Attila Kereszt, Szeged (HU); Michael Peter Gresshoff, Queensland (AU)

(73) Assignee: The University of Queensland, St. Lucia (AU)

(21) Appl. No.: 13/607,517

(22) Filed: Sep. 7, 2012

Related U.S. Application Data
(63) Continuation of application No. 12/158,300, filed on Nov. 25, 2008, now abandoned.

(30) Foreign Application Priority Data
Dec. 23, 2005 (AU) 2005907281

(51) Int. Cl. C12N 15/82

(52) U.S. Cl. CPC C12N 15/8262
USPC 800/278; 530/378; 536/23.6; 435/320.1; 800/298; 435/419; 435/468; 530/387.9

(57) ABSTRACT

The invention provides GmNFR1α, GmNFR1β, GmNFR5α, and GmNFR5β soybean nodulation factor receptor proteins, a receptor complex, and encoding nucleic acids. Also provided are GmNFR1α, GmNFR1β, GmNFR5α, and GmNFR5β promoters, which may be useful for expressing autologous or heterologous sequences in plants, such as soybean. Variant proteins and nucleic acids including RNA splice variants, mis-sense mutants, and non-sense mutants are also described. Also provided are genetically-modified plants and methods of producing genetically-modified plants. Over-expression of soybean nodulation factor receptor proteins by genetically-modified plants may lead to enhanced and/or otherwise facilitated nodulation and/or nitrogen fixation. Genetically-modified plants with down-regulated nodulation factor receptor expression, such as by RNAi or antisense constructs, may exhibit inhibited, diminished, or otherwise reduced nodulation and/or nitrogen fixation.

### CROSS-REFERENCE TO RELATED INVENTIONS

This application is a continuation of U.S. patent application Ser. No. 12/158,300, filed Nov. 25, 2008, which claims benefit of PCT/AU2006/001963, filed Dec. 23, 2006, which claims priority from Australia Patent Application 2005907281, filed Dec. 23, 2005, each of which are hereby incorporated by reference in their respective entireties.

### FIELD OF THE INVENTION

THIS INVENTION relates to plant proteins and encoding nucleic acids. More particularly, this invention relates to isolated nodulation receptor proteins and nucleic acids that may be useful in enhancing nodulation and/or nitrogen fixation in crop plants such as soybean (Glycine max L.).

### BACKGROUND OF THE INVENTION

Nodulation and symbiotic nitrogen fixation in legumes provide a major conduit for nitrogen into the earth's biosphere, capable of replacing synthetic fossil-fuel based fertilizer augmentation of high input food production (Gresshoff, 2003, Genome Biology 4, 201; Caetano-Anollés & Gresshoff, 1991, Annu. Rev. Microbiol. 45, 345).

The understanding and concomitant optimization of this symbiotic process of plant-bacterium interaction is gaining renewed emphasis with ever-increasing crude oil costs (above US $60 per barrel in late 2006).

Nodule ontogeny in legumes requires the reception of a Rhizobium-derived ‘Nodulation Factor’ (NF, a lipo-chito-oligosaccharide) presumably by a LysM-type receptor kinase complex comprised of NFR1 and NFR5 (Radutoiu et al., 2003, Nature 425, 585; Madsen et al., 2003, Nature 425, 637; Limpens. et al., 2003, Science 302, 630). “Rhizobium” refers to the generic term of root colonizing and nodulating bacteria. Soybean specifically is nodulated by Bradyrhizobium japonicum, Rhizobium fredii and Sinorhizobium strain NGR234.

NF perception leads to induction of cortical cell divisions (CCD), and in parallel, the deformation, curling and eventual invasion of root hairs permitting the entry of Rhizobium bacteria, and enrichment of NF signalling (Gresshoff, 2003, supra; Caetano-Anollés & Gresshoff, 1991, supra; Oldroyd, 2001, Annals of Botany 87, 709).

The NF receptor genes of soybean, a major legume for food, industry and medical application, remained hitherto undefined.

### SUMMARY OF THE INVENTION

The invention is therefore broadly directed to isolated plant nodulation factor receptor proteins and encoding isolated nucleic acids and/or their use in improving, enhancing and/or otherwise facilitating nodulation in plants.

In one preferred form the invention provides a soybean nodulation factor receptor protein and encoding isolated nucleic acid.

In a first aspect, the invention provides an isolated protein comprising an amino acid sequence set forth in SEQ ID NO:1, SEQ ID NO:2, SEQ ID NO:3, or SEQ ID NO:4.

This aspect also includes fragments, variants and derivatives of said isolated protein.

In a second aspect, the invention provides an isolated nodulation factor receptor complex comprising a plurality of nodulation factor receptor proteins.

In a third aspect, the invention provides an isolated nucleic acid that encodes the isolated protein of the first aspect.

In particular embodiments, the isolated nucleic acid comprises a nucleotide sequence set forth in any one of SEQ ID NOS:5-12.

This aspect also includes fragments and variants of said isolated nucleic acid.

Furthermore, this aspect of the invention extends to an isolated nodulation factor gene and/or genetic components thereof including but not limited to one or more introns, one or more exons, a promoters, a 5′ untranslated region and a 3′ untranslated region.

In a fourth aspect, the invention provides an isolated nucleic acid comprising a promoter-active fragment of a nodulation factor receptor gene.

Preferably, the promoter-active fragment is a fragment of a nucleotide sequence set forth in any one of set forth in any one of SEQ ID NOS:5-8.

In particular embodiments, the promoter-active fragment comprises a nucleotide sequence set forth in any one of set forth in any one of SEQ ID NOS:13-16.

In a fifth aspect, the invention provides a chimeric gene comprising the promoter-active fragment of the fourth aspect and a heterologous nucleic acid.

In a sixth aspect, the invention provides a genetic construct comprising the isolated nucleic acid of the third aspect or the chimeric gene of the fourth aspect.

Preferably, the genetic construct is an expression construct, wherein the isolated nucleic acid or the chimeric gene is operably linked or connected to one or more regulatory sequences in an expression vector.

In a seventh aspect, the invention provides a genetically-modified plant comprising the genetic construct of the sixth aspect.

In an eighth aspect, the invention provides a method of producing genetically-modified plant, plant cell or tissue including the step of introducing the genetic construct of the sixth aspect into a plant cell or tissue to thereby genetically-modify said plant cell or tissue.

In one embodiment, the genetically-modified plant, plant cell or tissue stably expresses a recombinant nodulation factor receptor protein.

Preferably, the genetically-modified plant, plant cell or tissue displays relatively improved, enhanced and/or otherwise facilitated nodulation and/or nitrogen fixation.

In another embodiment, the genetically-modified plant, plant cell or tissue expresses a nodulation factor receptor RNAi or antisense construct.

Preferably, the genetically-modified plant tissue displays relatively inhibited, diminished or otherwise reduced nodulation and/or nitrogen fixation.

In a ninth aspect, the invention provides a method of modulating nodulation in a plant including the step of introducing the genetic construct of the sixth aspect into a plant.

In one embodiment, the genetically-modified plant, plant cell or tissue displays relatively improved, enhanced and/or otherwise facilitated nodulation and/or nitrogen fixation.

In an alternative embodiment, the genetically-modified plant, plant cell or tissue displays relatively inhibited, diminished or otherwise reduced nodulation and/or nitrogen fixation.

In a tenth aspect, the invention provides a host cell comprising the genetic construct of the sixth aspect.

In one embodiment, the host cell is derived, isolated or otherwise obtained from a genetically modified plant.

In another embodiment, the host cell is a cell into which the genetic construct has been introduced in vitro.

In an eleventh aspect, the invention provides an antibody which binds the isolated protein of the first aspect.

The antibody may be a monoclonal antibody or a polyclonal antibody.

Throughout this specification, unless otherwise indicated, “comprise”, “comprises” and “comprising” are used inclusively rather than exclusively, so that a stated integer or group of integers may include one or more other non-stated integers or groups of integers.

### BRIEF DESCRIPTION OF THE FIGURES

FIG. 1: Symbiotic phenotypes of soybean non-nodulation mutants nod49 and rj1

A) Eight week-old plants grown without added nitrogen fertilizer, and inoculated with B. japonicum CB1809 showing the growth and nitrogen deficiency related phenotype caused by the absence of nodulation in mutants rj1, nod49, and nod139. rj1 is a naturally occurring non-nodulation mutant of soybean often used for the evaluation of nitrogen input into soybean cropping systems (6). Bragg and Clark are wild types. rj1/Clark and Bragg/nod49 are near-isogenic pairs; nod139 is an independent non-nodulation locus (15) mutated in GmNFR1α and GmNFR1β.

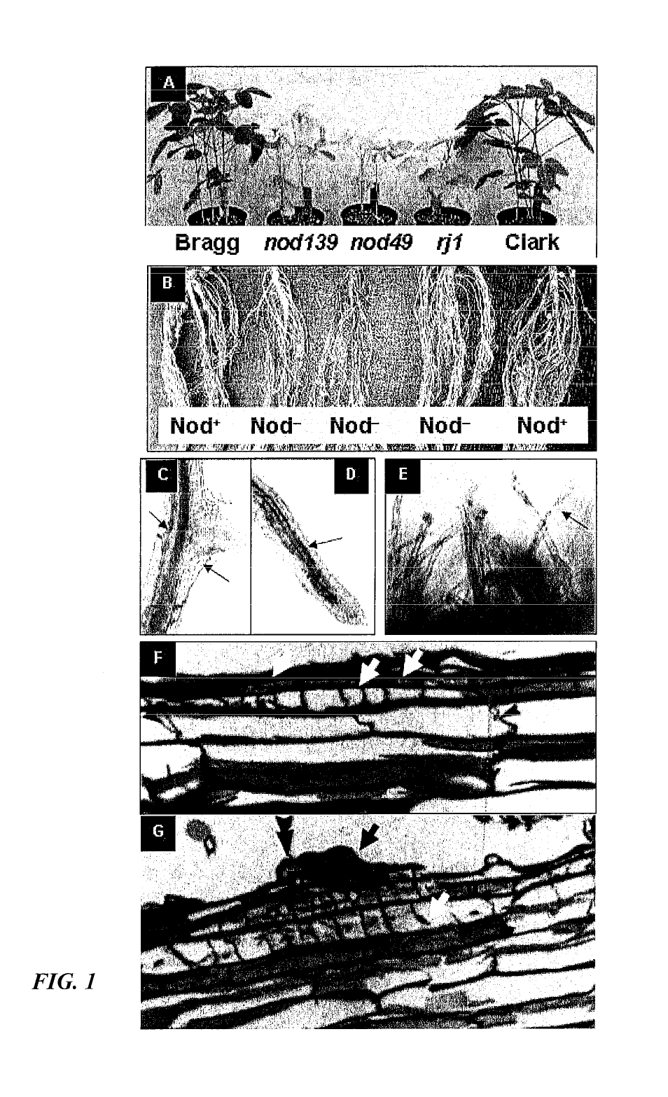

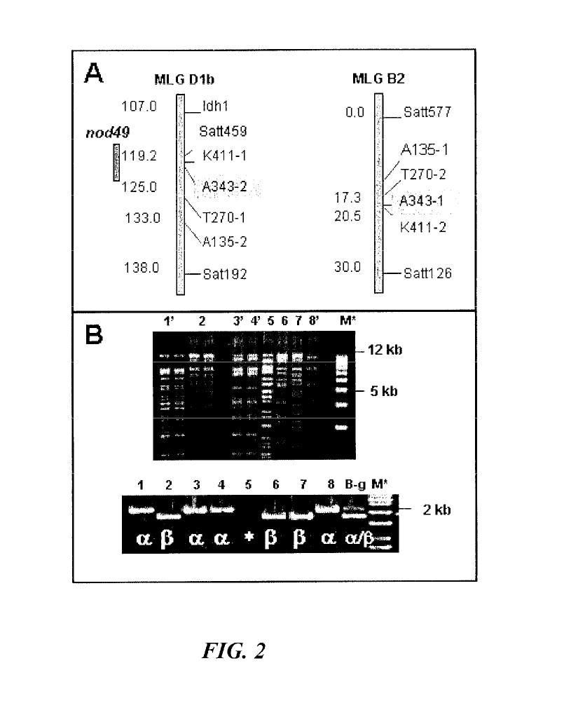

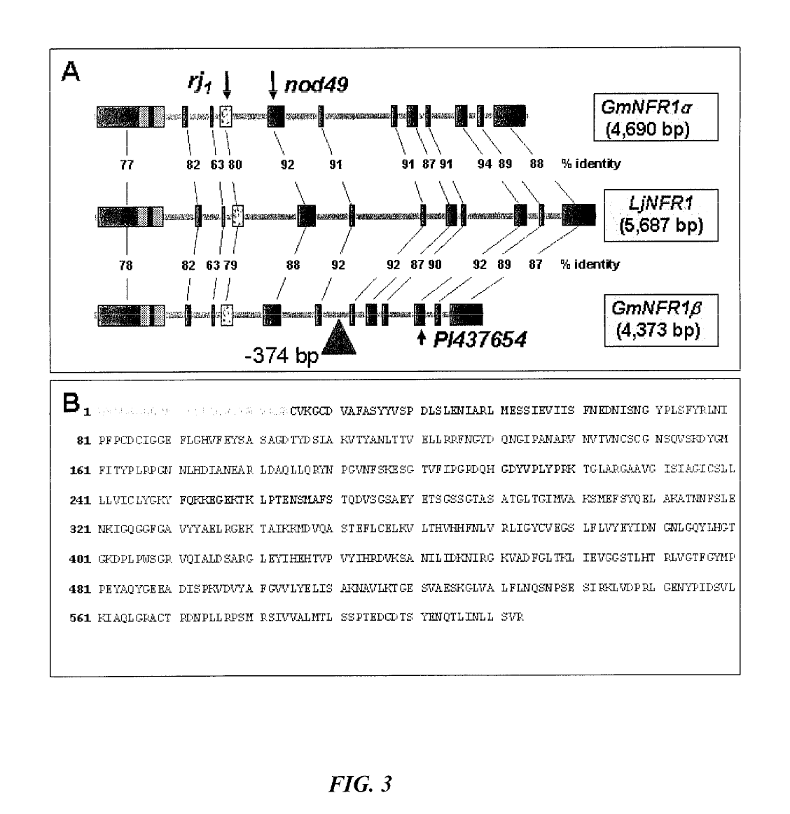

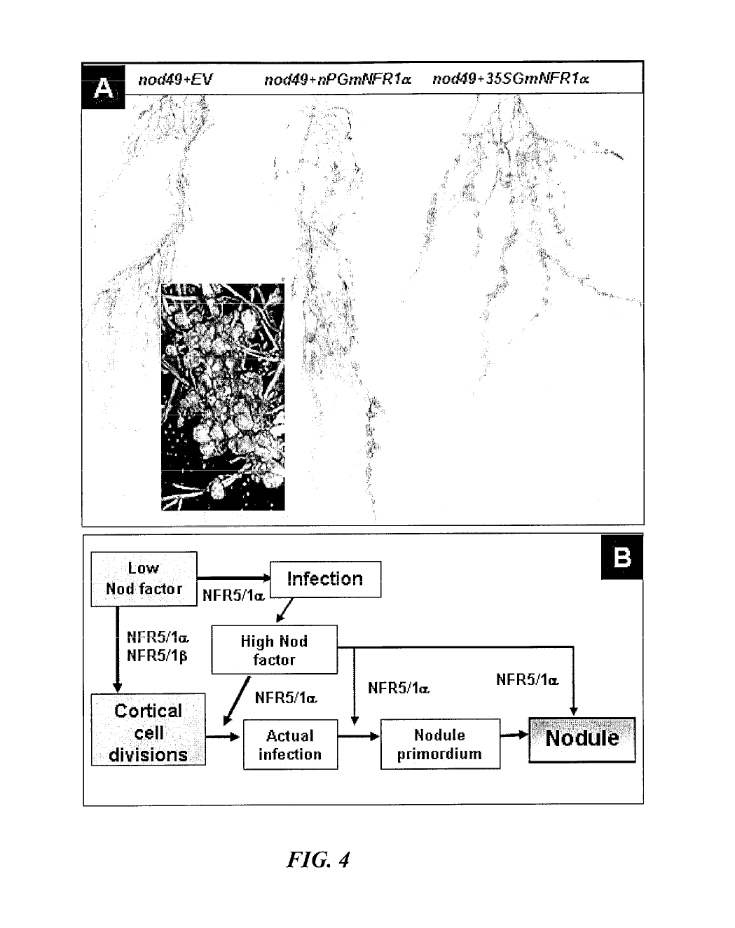

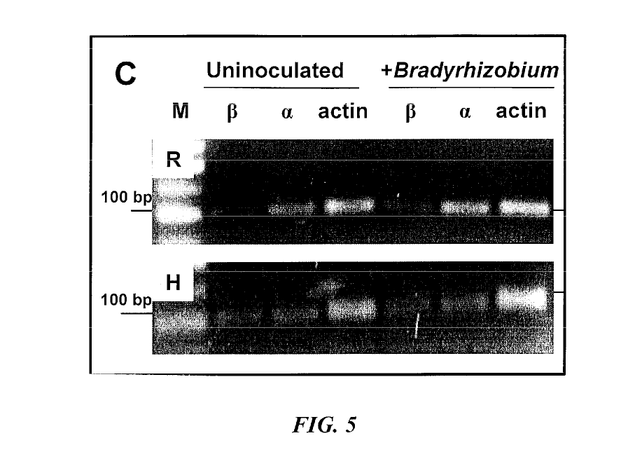

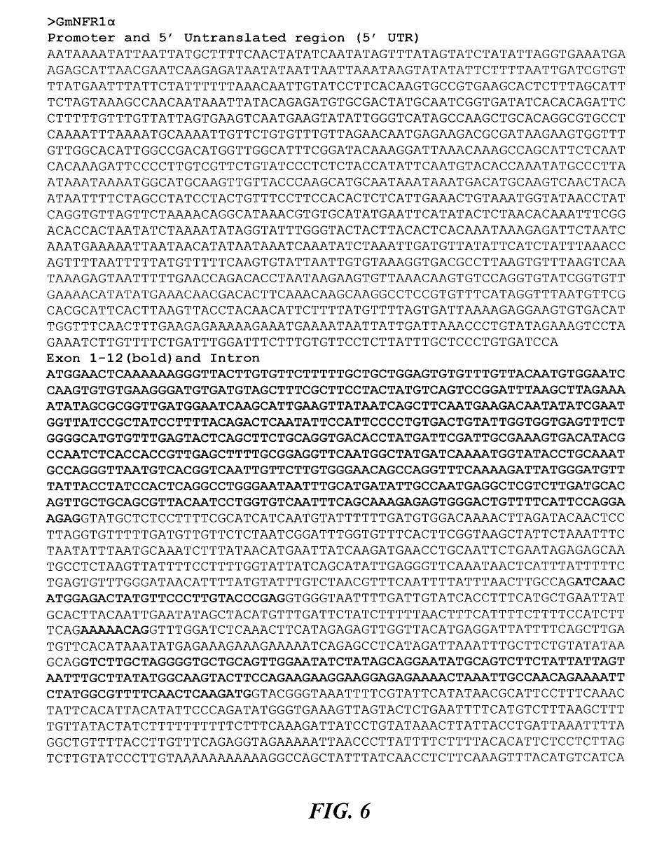

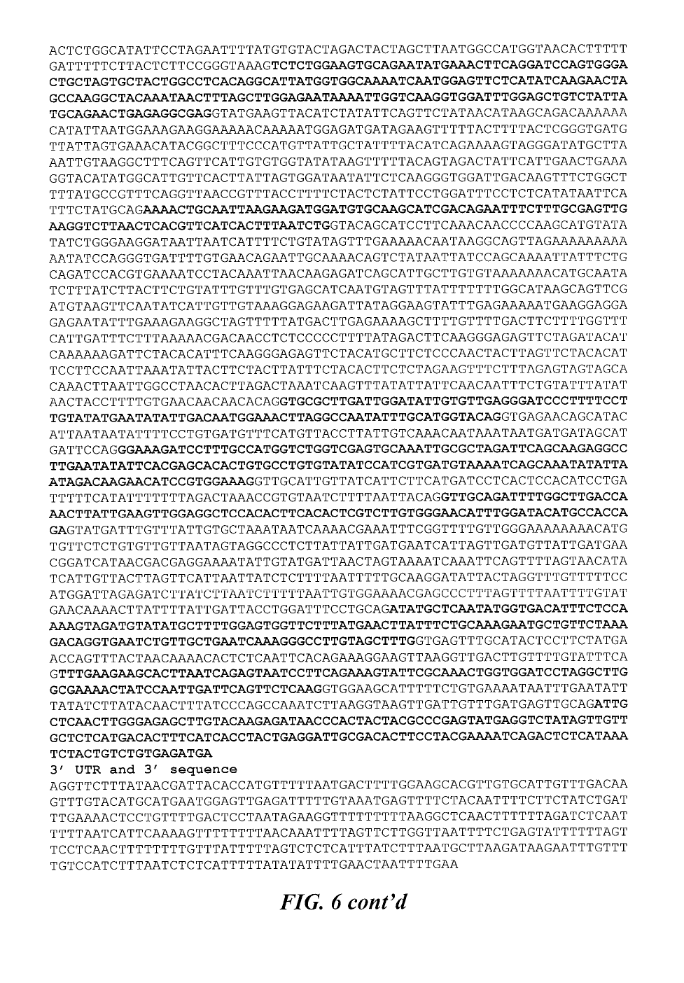

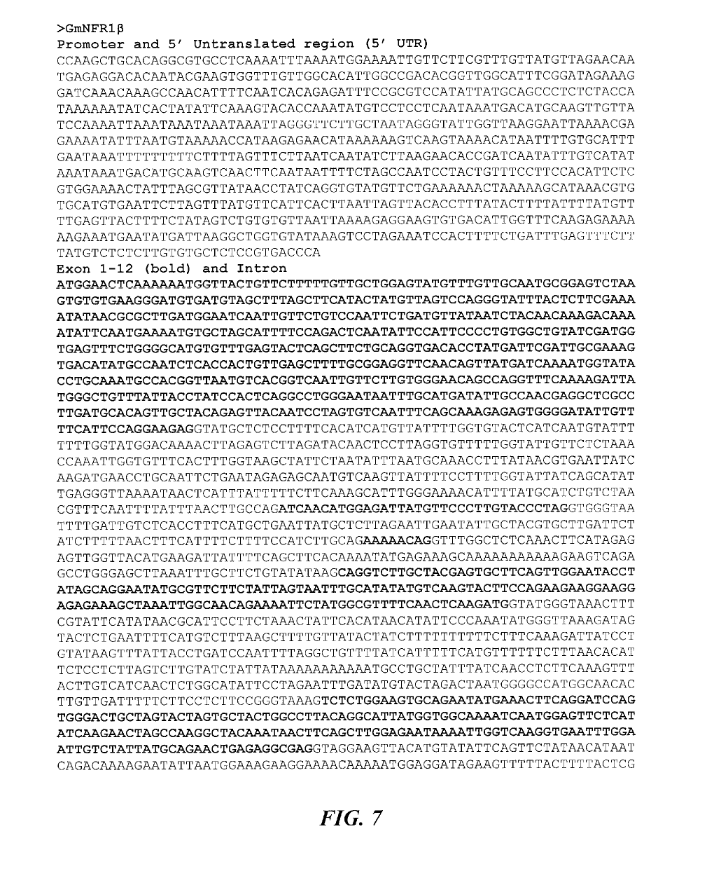

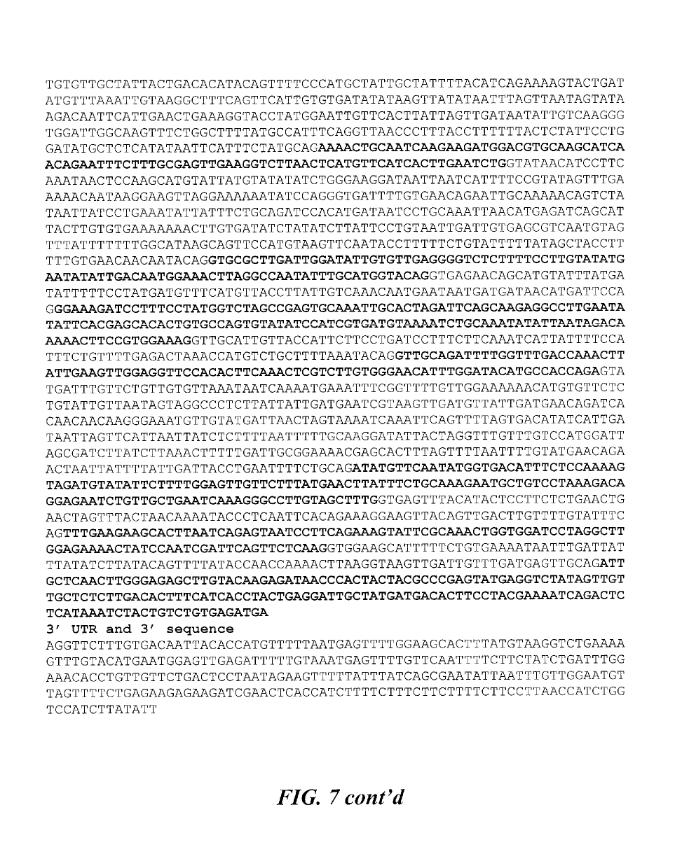

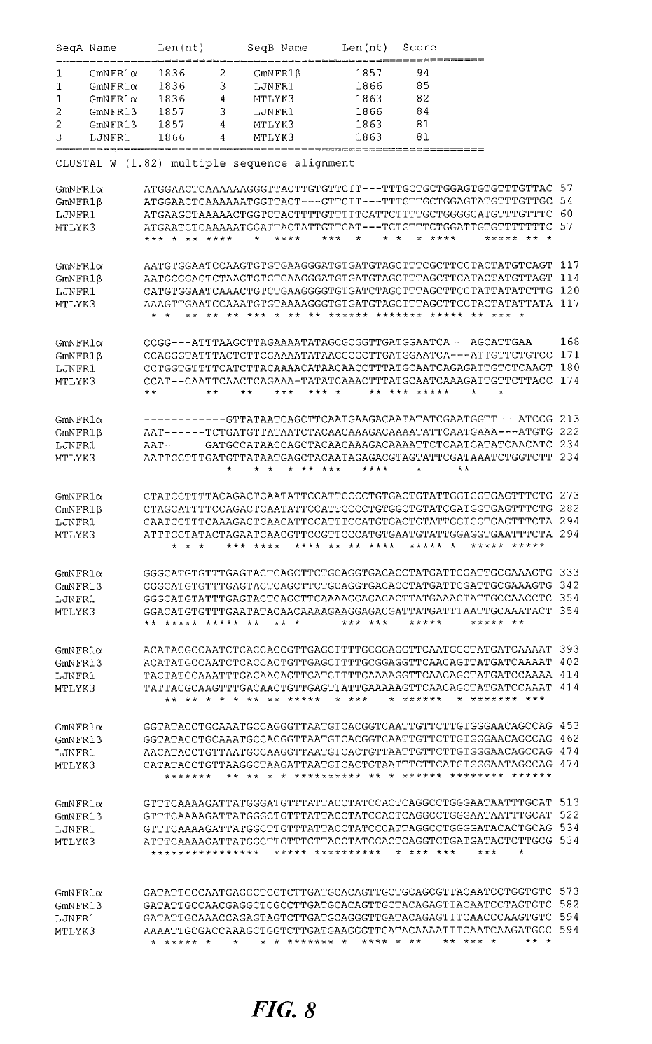

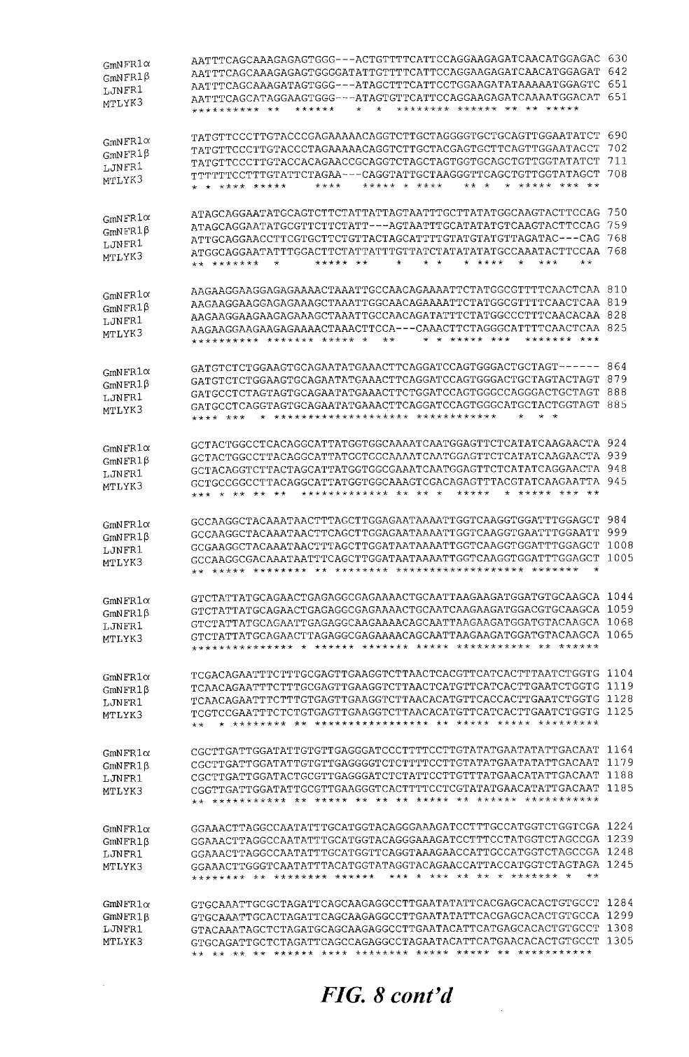

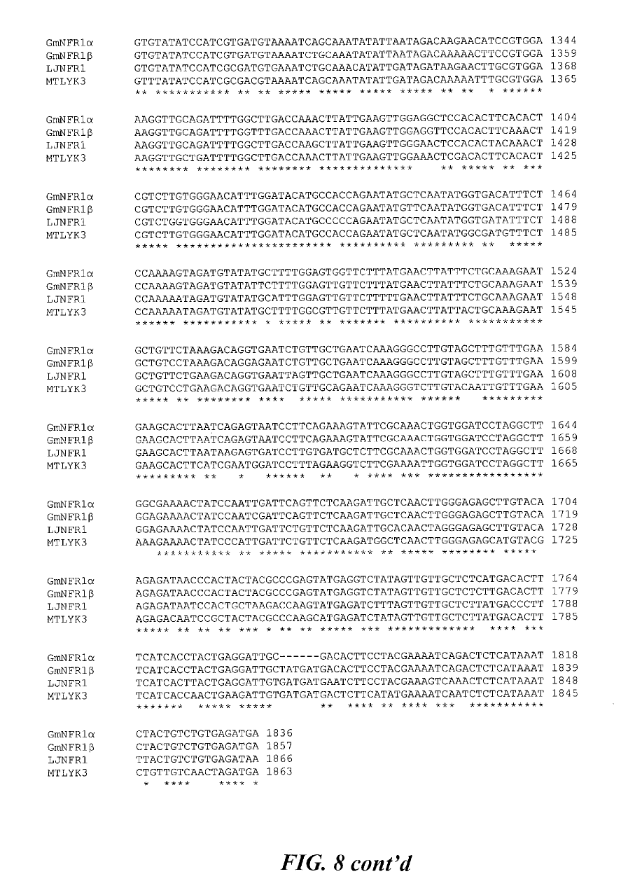

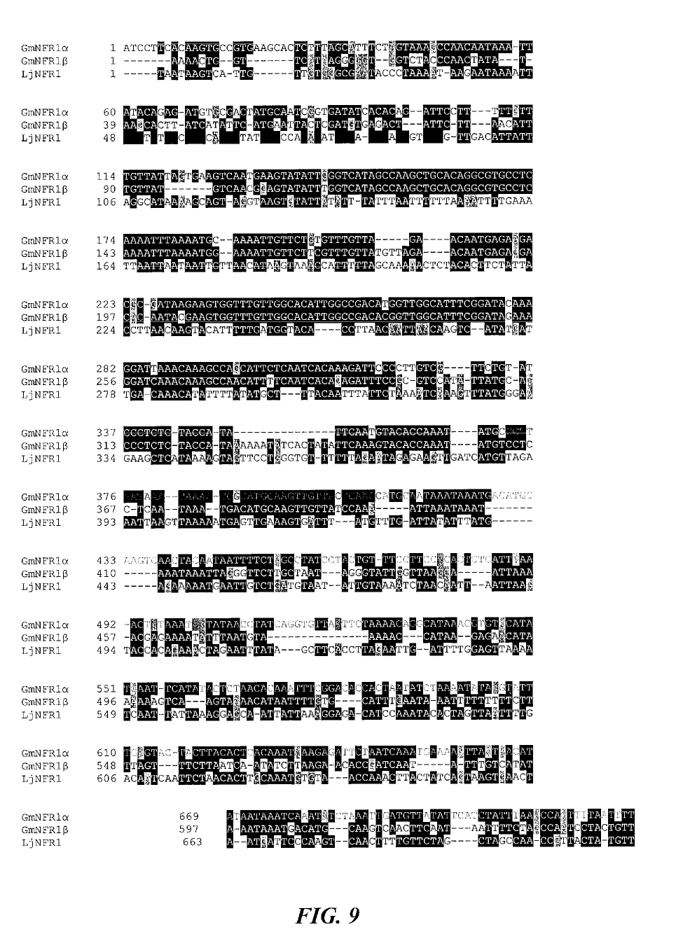

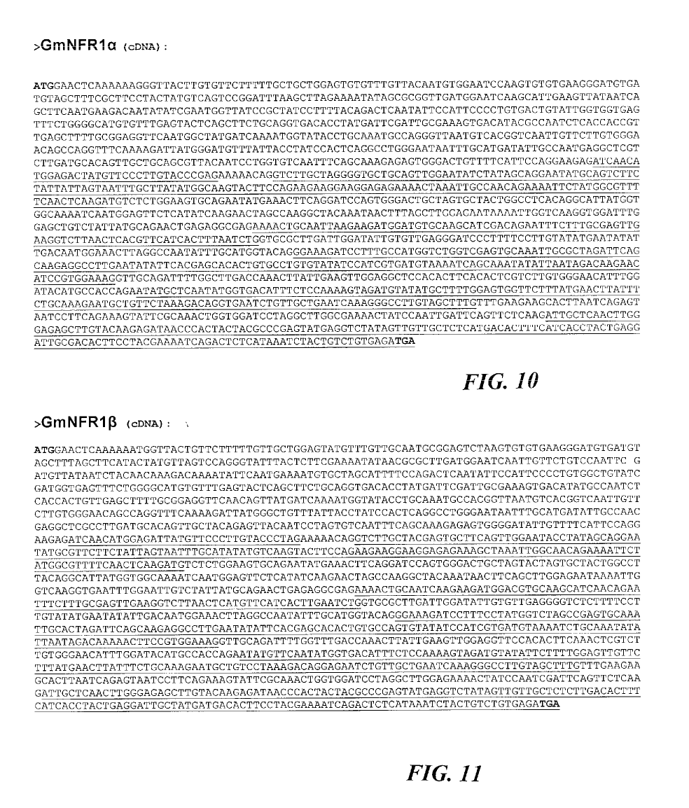

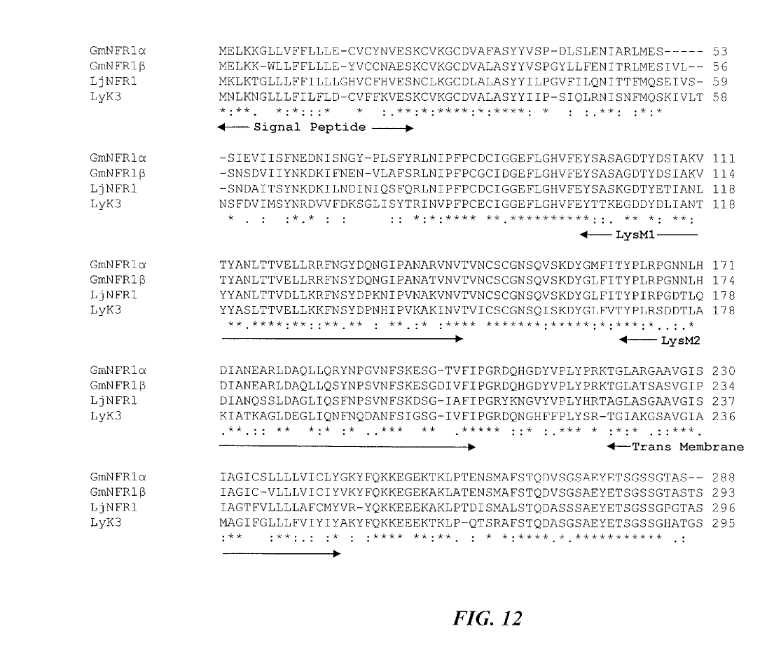

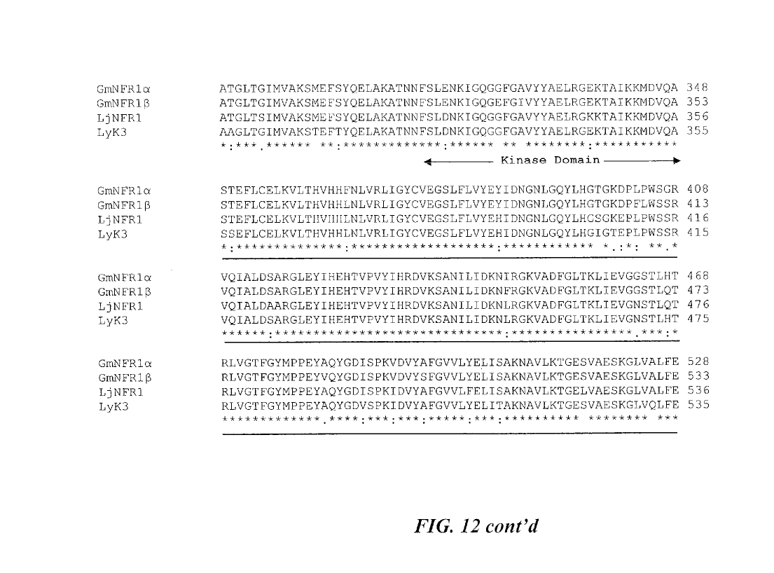

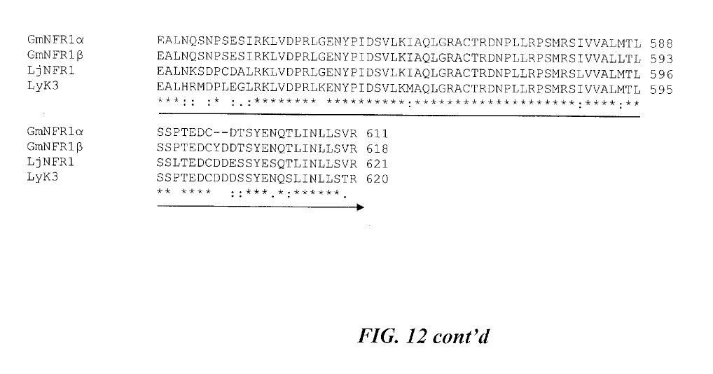

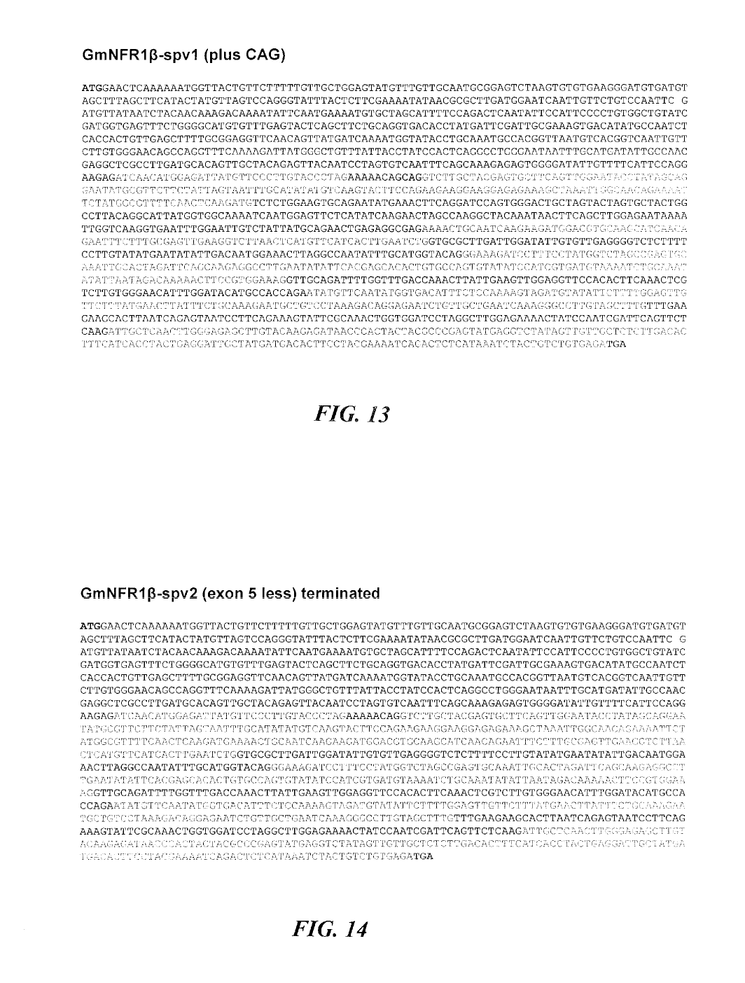

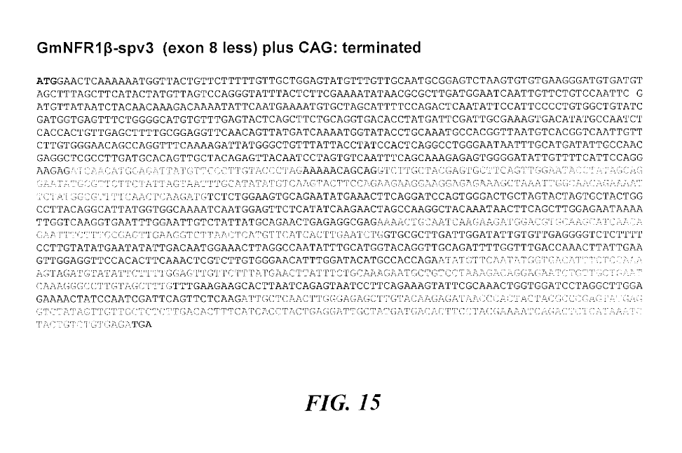

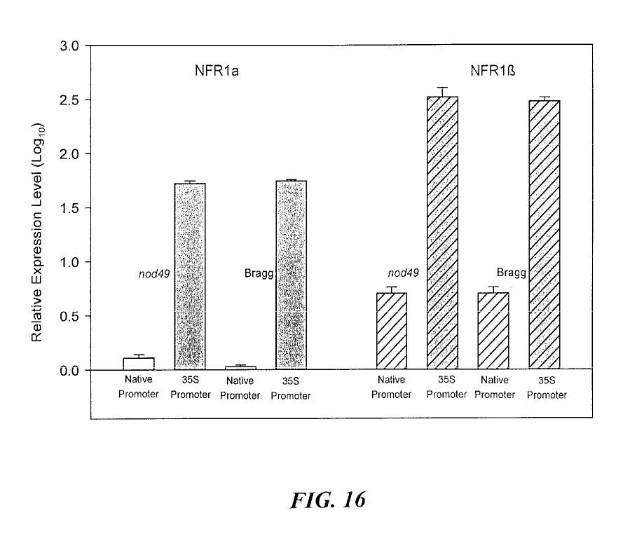

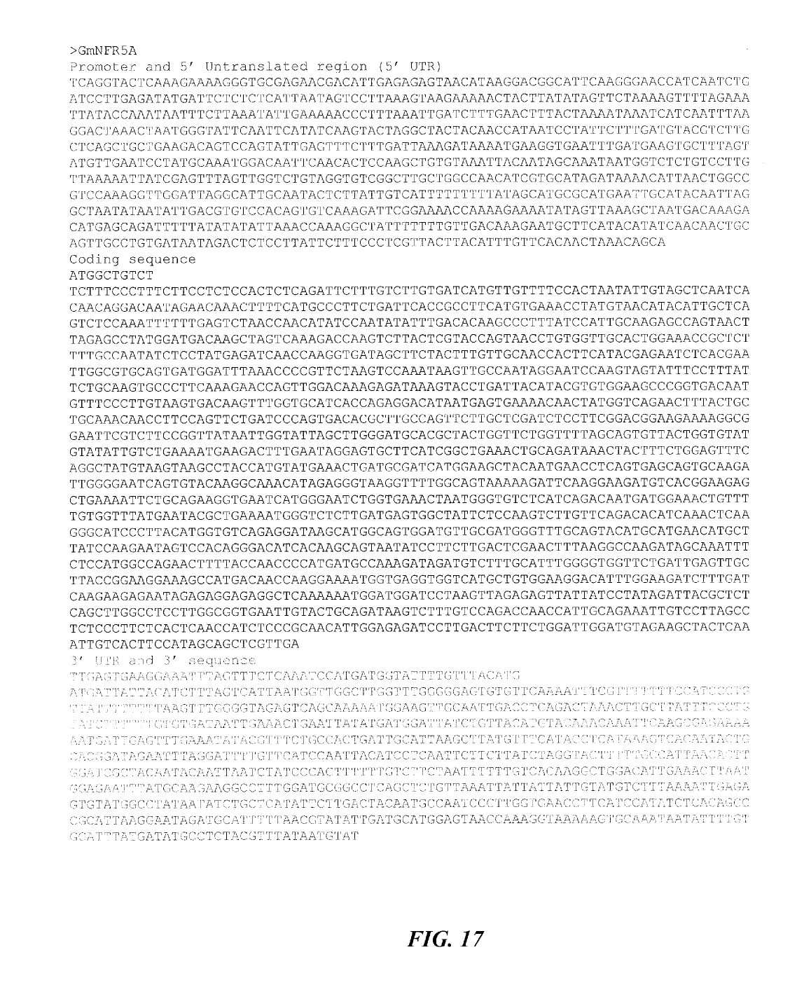

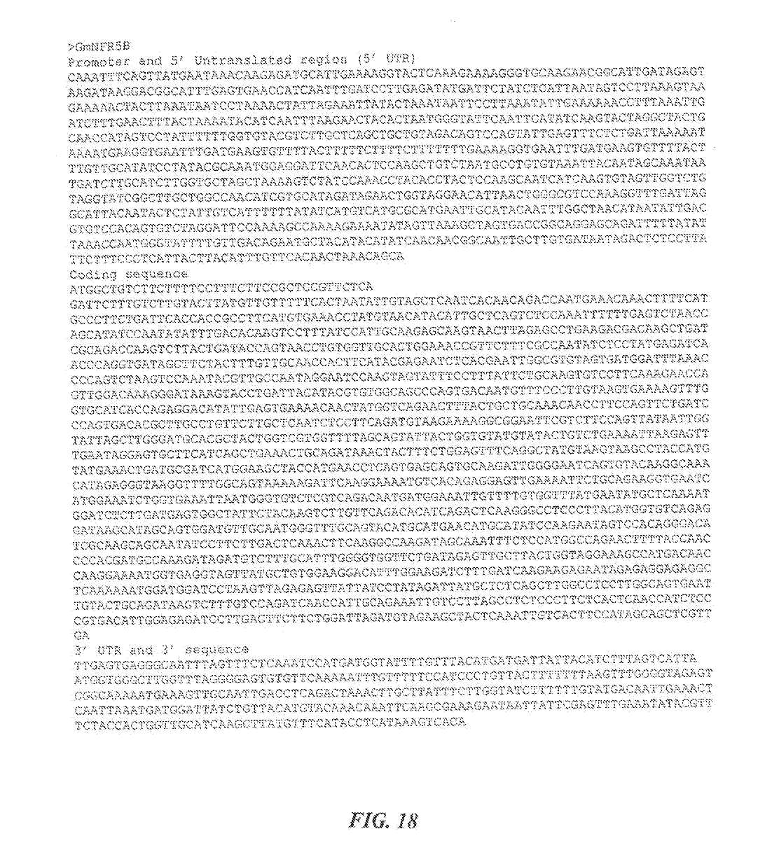

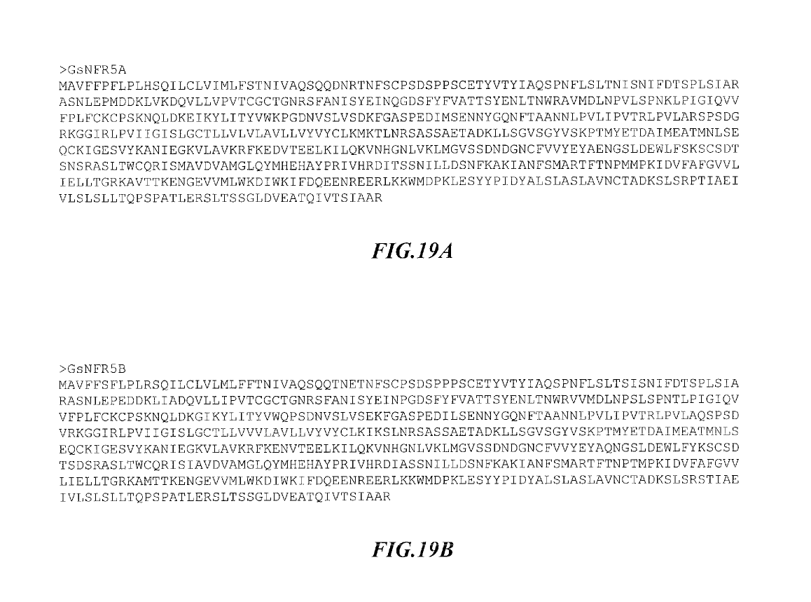

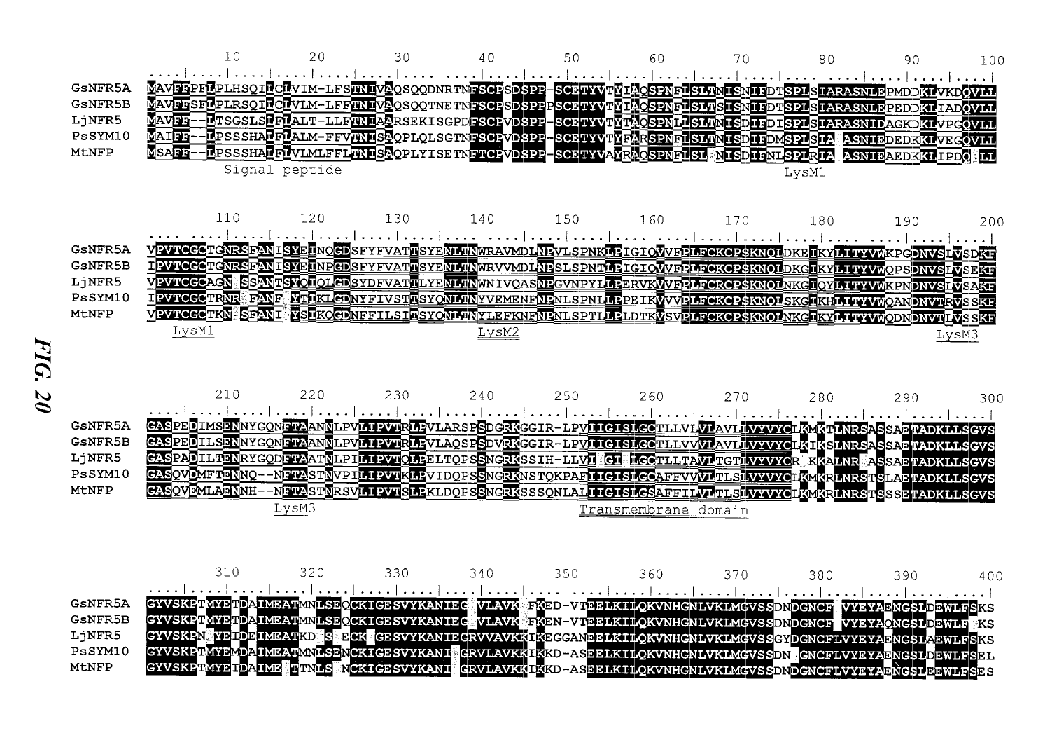

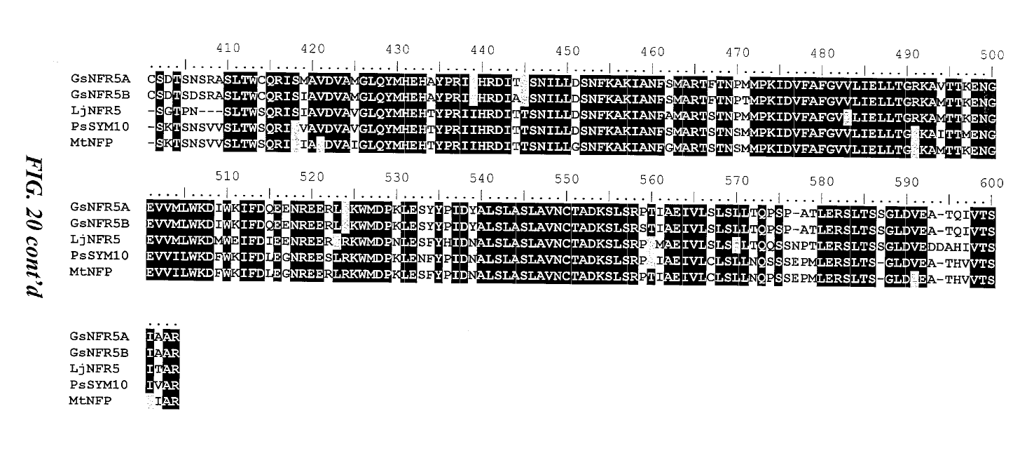

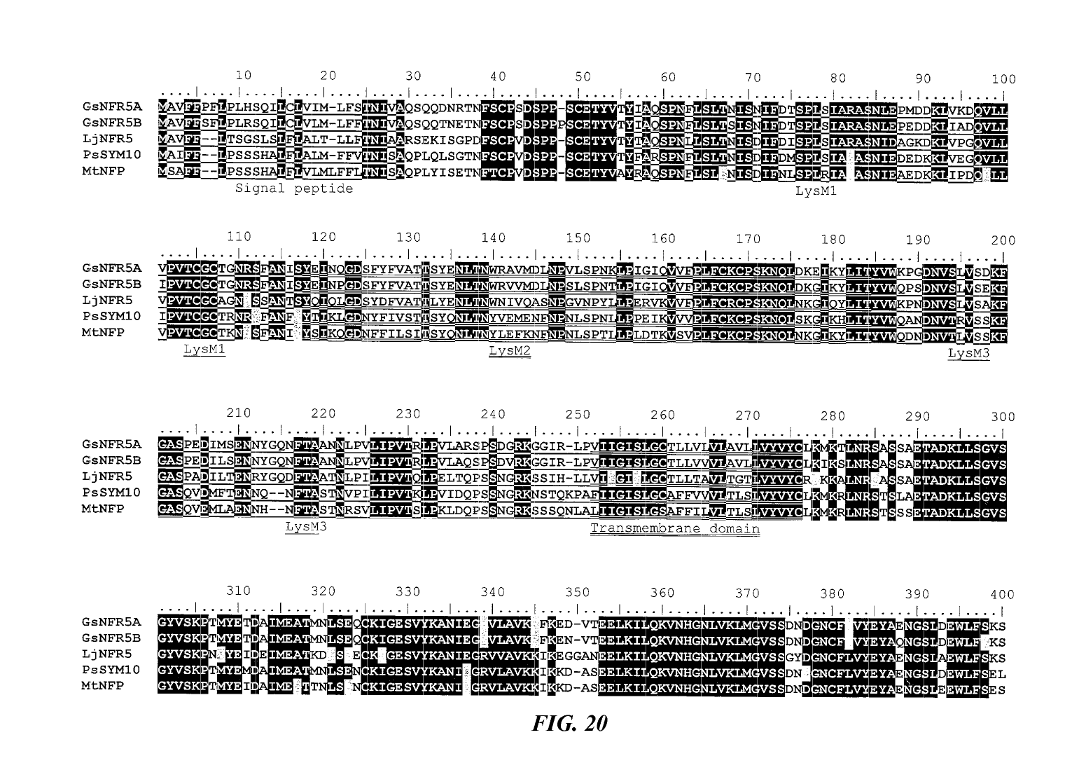

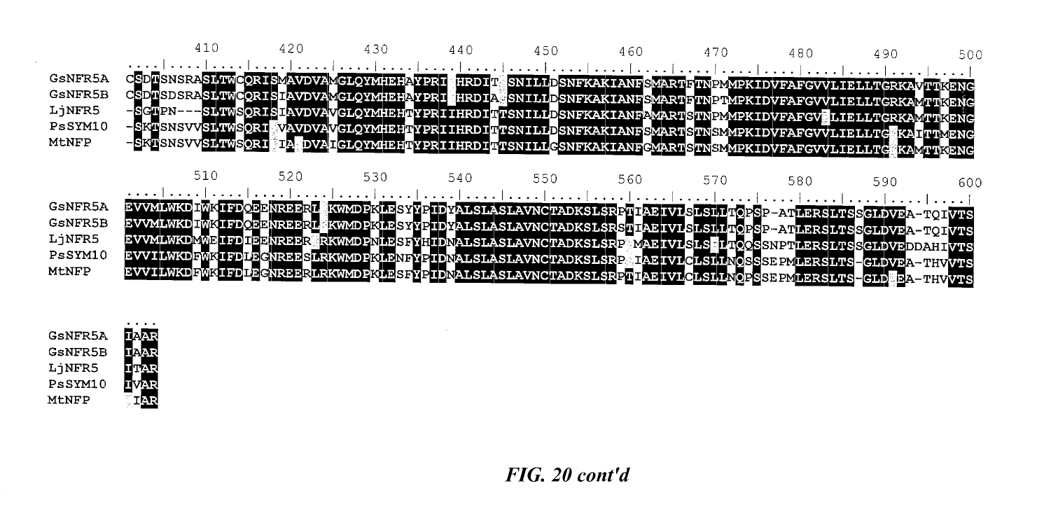

B) Root systems of plants shown in FIG. 1A illustrating mutant non-nodulating phenotypes.

C) Mycorrhizal root of nod49 (arrow shows external hyphae and internally infected cells).

D) Mycorrhizal root of rj1 (note that the outer cortex and root tip region are not infected).

E) Absence of root hair curling and deformation in nod49 inoculated with a total of 10⁸ cells of B. japonicum USDA110 per seedling.

F) Section of a wild-type Bragg root inoculated with B. japonicum USDA110 showing sub-epidermal cortical cell division (CCD; see arrow; also referred to as ‘pseudoinfections’ (13). Mutants nod49 and rj1 achieve this stage but fail to precede further (12). nod139 does not achieve this stage.

G) Section of a soybean Bragg root inoculated with B. japonicum showing an early cell division cluster associated with a successful infection event (a markedly curled and infected root hair; see arrow; labeled ‘actual infections’ (13)). This stage is not observed in nod49 or rj1.

FIG. 2: Isolation of the GmNFR1 genes

A) Map position of the nod49 mutation. Marker Satt459 cosegregated with the non-nodulation phenotype in a G. max nod49×Glycine soja CI 111070 F2 population. DNA sequences of closely linked RFLP markers K411-1 and A343-2 had high identity to LjNFR1. A syntenic region involving at least four markers was found on MLG b2.

B) Fingerprinting of eight selected BAC clones from G. max PI437.654 (Clemson University Genomics Institute) identified with filter hybridization to a GmNFR1α probe (anchored by K411-1 and A343-2).

upper B panel: HindIII BAC fingerprinting of positive clones. BACs 1, 3, 4 and 8 are part of one contig; BACs 2, 6, and 7 from another contig. BAC 5 was a false positive. BACs 1 (BAC54B21) and 2 (BAC55N1) were run as duplicate lanes.

lower B panel: Verification of LysM type RK probe, used to isolate BAC clones as two differently sized PCR products (α and β) correlates with separate BAC contigs. B-g=Bragg genomic DNA.

FIG. 3: Structure of the soybean GmNFR1 genes and the gene product

A) Genomic organization of the GmNFR1α and β genes compared to that of LjNFR1 (2). Numbers indicate the nucleotide sequence identity between exons. Locations of nucleotide changes in nod49, rj1 and PI437.654 are indicated; a 374 bp deletion in intron 6 of GmNFR1β did not affect the ORF and presence of its mRNA.

B) The predicted amino acid sequence of GmNFR1α; key regions are highlighted (blue=LysM domains; green=signal peptide (SP); red=transmembrane domain (TMD); purple=protein kinase domain (PKD). Note: charged domains on either side of the TMD. Multiple Sequence Alignment of GmNFR1α, GmNFR1β, MtLYK3, and LjNFR1 proteins is shown in Supplementary Material. Cleavage of the signal peptide is between the ESK and CV residues according to the Signal P program.

FIG. 4:

A) Complementation of nod49 non-nodulation phenotype by wild-type GmNFR1α using hairy root transformation

Transformed root systems were scored 35 days after inoculation with B. japonicum CB 1809. Left: Transgenic roots of nod49 transformed with Agrobacterium rhizogenes strain K599 carrying the empty vector pCAMBIA1305.1 (in which case all roots were scored); Middle: root system of nod49 transformed with K599 carrying full length GmNFR1α cDNA behind its own 3.4 kb native promoter. Full length cDNA was obtained by PCR from a root cDNA library of Bragg. For nodulated test, only nodulated roots (average 40% of all developed roots) were scored as many roots were deemed to be escapes, incomplete transfers, or silenced roots; Right: root system of nod49 transformed with K599 carrying full length GmNFR1α cDNA driven by the 35S promoter of CaMV. Note the extended nodulation interval as most parts of the roots are nodulated and the clustered nodules along upper root regions or rootlets (see insert).

B) Model of nodulation factor (NF) perception in soybean: NF perception is required at several stages of the nodule ontogeny with early infection events responding differently than cortical and presumably pericycle cell divisions. GmNFR1α, presumably in partnership with GmNFR5, is capable of fulfilling all functions and is thus similar to LjNFR1. GmNFR1β lacks the ability to perceive NF at low Bradyrhizobium titers, yet suffices for the induction of cortical cell divisions (CCDs; c.f., FIG. 1F). Actual infections are combinations of successful infection threads and CCDs (c.f., FIG. 1G). Infections mediated by GmNFR1α allow the enrichment of rhizobia and NF leading to subsequent maintenance of CCDs and concomitant pericycle cell divisions. ‘Low’ and ‘High Nod factor’ refers to presumed local concentrations. Grey shaded boxes are the terminal symbiotic stages achieved in mutants nod49 and rj1 (12), whereas wild type or complemented plants progress.

FIG. 5:

RT-PCR determination of transcription activity of GmNFR1α/β in both root and hypocotyls of either inoculated or uninoculated wild type Bragg soybean plants (14 days after inoculation with Bradyrhizobium japonicum CB1809). Transcript levels in mutant nod49 are equivalent. Soybean Actin 2/7 was used as control.

FIG. 6:

GmNFR1α nucleotide sequence including 5′ UTR comprising a promoter region, a coding sequence and a 3′ UTR. Exons are bolded.

FIG. 7:

GmNFR1β nucleotide sequence including 5′ UTR comprising a promoter region, a coding sequence and a 3′ UTR. Exons are bolded.

FIG. 8:

GmNFR1α and GmNFR1β nucleotide sequence homology. ClustalW alignment of GmNFR1α and GmNFR1β coding sequences with LjNFR1 and MtLyK3 coding sequences.

FIG. 9:

Promoter sequence alignment of GmNFR1α, GmNFR1β and LjNFR1

FIG. 10:

Exon boundaries of GmNFR1α coding sequence. Exon sequences are bolded.

FIG. 11:

Exon boundaries of GmNFR1β coding sequence. Exon sequences are bolded.

FIG. 12:

Alignment of GmNFR1α and GmNFR1β amino acid sequences. GmNFR1α and GmNFR1β amino acid sequence are aligned with LjNFR1 and MtLYK3 amino acid sequences.

FIG. 13:

GmNFR1β-spv1 splice variant (plus CAG). The additional CAG codon is derived from the 5′ end of intron 3 utilising the nearby AG splice site. The small size of exon 3 may be the cause of instability.

FIG. 14:

GmNFR1β-spv2 splice variant (exon 5 less) terminated.

FIG. 15:

GmNFR1β-spv3 splice variant (exon 8 less) terminated.

FIG. 16:

Relative expression level of the GmNFR1 genes in the transgenic roots The expression level achieved by the different constructs is compared to that of roots transformed with the empty vector.

FIG. 17:

GmNFR5α nucleotide sequence including 5′ UTR comprising a promoter region, a coding sequence and a 3′ UTR.

FIG. 18:

GmNFR5β nucleotide sequence including 5′ UTR comprising a promoter region, a coding sequence and a 3′ UTR.

FIG. 19:

A) Amino acid sequence of GmNFR5α protein and

B) Amino acid sequence of GmNFR5β protein.

FIG. 20:

Amino acid sequence alignment of GmNFR5α, GmNFR5β, LjNFR1, and MtLYK3 proteins.

### BRIEF DESCRIPTION OF THE SEQUENCE LISTING

SEQ ID NO:1 GmNFR1α protein amino acid sequence.
SEQ ID NO:2 GmNFR1β protein amino acid sequence.
SEQ ID NO:3 GmNFR5α protein amino acid sequence.
SEQ ID NO:4 GmNFR5β protein amino acid sequence.
SEQ ID NO:5 GmNFR1α nucleotide sequence comprising 5′ untranslated, coding sequence, and 3′ untranslated sequence.
SEQ ID NO:6 GmNFR1β nucleotide sequence comprising 5′ untranslated, coding sequence, and 3′ untranslated sequence.
SEQ ID NO:7 GmNFR5α nucleotide sequence comprising 5′ untranslated, coding sequence, and 3′ untranslated sequence.
SEQ ID NO:8 GmNFR5β nucleotide sequence comprising 5′ untranslated, coding sequence, and 3′ untranslated sequence.
SEQ ID NO:9 GmNFR1α coding sequence.
SEQ ID NO:10 GmNFR1β coding sequence.
SEQ ID NO:11 GmNFR5α coding sequence.
SEQ ID NO:12 GmNFR5β coding sequence.
SEQ ID NO:13 GmNFR1α 5′ untranslated sequence comprising promoter-active region.
SEQ ID NO:14 GmNFR1β 5′ untranslated sequence comprising promoter-active region sequence.
SEQ ID NO:15 GmNFR5α 5′ untranslated sequence comprising promoter-active region.
SEQ ID NO:16 GmNFR5β 5′ untranslated sequence comprising promoter-active region.
SEQ ID NO:17 GmNFR1β-spv1 splice variant (plus CAG)
SEQ ID NO:18 GmNFR1β-spv2 splice variant (exon 5 less) terminated.
SEQ ID NO:19 GmNFR1β-spv3 splice variant (exon 8 less) terminated.
SEQ ID NOS:20-53 Miscellaneous GmNFR1α and GmNFR1β primer sequences.
SEQ ID NOS:54-75 Miscellaneous GmNFR5α and GmNFR5β primer sequences.

### DETAILED DESCRIPTION OF THE PREFERRED EMBODIMENT

Increased abundance of Nod factor in normal soybean decreases the effect of environmental stress agents such as high temperature, soil nitrate, and acidity (but not salinity). This suggests that these stresses act by decreasing the plant's ability to transmit the Nod factor signal. Similarly, Nod factor treatment of soybean induces disease resistance in some cases.

The present invention is predicated on the discovery of Nod factor receptor genes (GmNFR1α and ft GmNFR5α and β) and their respective native promoters in soybean and demonstration that increased nodulation coupled with nitrogen gain (and potential yield) occurs after over-expression of the receptor protein GmNFR1α in soybean. It is also contemplated that over-expressing both GmNFR1α and GmNFR5α proteins together may further increase nodulation and nitrogen fixation of soybean plants.

The invention therefore provides means for increasing soybean nitrogen fixation, increasing seed and oil production, assisting establishment in low Bradyrhizobium soils, nodulation under environmental stress situations, optimization of bacterial host range and associated alleviation of bacterial competition for nodulation sites on soybean roots and increased resistance to pathogenic bacteria and fungi.

Control of specific ligand (i.e., nod factor) perception to control cell division initiation in a plant provides a unique tool, particularly with regard to major grain legumes of importance in countries such as USA, Brazil, China, Argentina, and India.

It is also contemplated that in light of nodulation factor receptor genes being involved in bacterial signal recognition that they may also play a role in plant pathogen interactions and that knowledge of the soybean components may lead to improved plant health through manipulation of LysM type receptor proteins.

As used herein, nodulation factor receptor proteins of Glycine max are generically referred to as “GmNFR” proteins.

Accordingly, nodulation factor receptor genes and nucleic acids of Glycine max are generically referred to as “GmNFR” genes or nucleic acids.

By “gene” is meant a structural unit of a genome, which comprises one or more genetic elements such as a protein-coding nucleotide sequence, translation start and stop codons, exons, introns, a promoter, a 5′ unstranslated region (5′UTR), a 3′ unstranslated region (3′UTR), and a polyadenylation (polyA) sequence, although without limitation thereto. It will also be appreciated that not all of these genetic elements are necessarily present in a particular gene.

Accordingly, isolated GmNFR nucleic acids of the invention comprise a nucleotide sequence of, or complementary to, a GmNFR gene sequence or genetic element thereof.

In one embodiment, the invention provides an isolated protein comprising an amino acid sequence set forth in SEQ ID NO: 1, referred to herein as a GmNFR1α protein.

The invention also provides an isolated GmNFR1α nucleic acid (SEQ ID NO:5) which comprises:
(i) a nucleotide sequence encoding said GmNFR1α protein (SEQ ID NO:9); and
(ii) a 5′ untranslated nucleotide sequence comprising a promoter-active region (SEQ ID NO:13).

The GmNFR1α nucleic acid also comprises a 3′ untranslated region.

In another embodiment, the invention provides an isolated protein comprising an amino acid sequence set forth in SEQ ID NO: 2, referred to herein as a GmNFR1β protein.

The invention also provides an isolated GmNFR1β nucleic acid (SEQ ID NO:6) which comprises:
(i) a nucleotide sequence encoding said GmNFR1β protein (SEQ ID NO:10); and
(ii) a 5′ untranslated nucleotide sequence comprising a promoter-active region (SEQ ID NO:14).

The GmNFR1β nucleic acid also comprises a 3′ untranslated region.

In yet another embodiment, the invention provides an isolated protein comprising an amino acid sequence set forth in SEQ ID NO: 3, referred to herein as a GmNFR5α protein.

The invention also provides an isolated GmNFR5α nucleic acid (SEQ ID NO:7) which comprises:
(i) a nucleotide sequence encoding said GmNFR1β protein (SEQ ID NO:11); and
(ii) a 5′ untranslated nucleotide sequence comprising a promoter-active region (SEQ ID NO:15).

The GmNFR5α nucleic acid also comprises a 3′ untranslated region.

In yet another embodiment, the invention provides an isolated protein comprising an amino acid sequence set forth in SEQ ID NO: 4, referred to herein as a GmNFR5β protein.

The invention also provides an isolated GmNFR5β nucleic acid (SEQ ID NO:8) which comprises:
(i) a nucleotide sequence encoding said GmNFR1β protein (SEQ ID NO:12); and
(ii) a 5′ untranslated nucleotide sequence comprising a promoter-active region (SEQ ID NO:16).

The GmNFR5β nucleic acid also comprises a 3′ untranslated region.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material includes material in native and recombinant form.

The term “nucleic acid” as used herein designates single or double stranded mRNA, RNA, cRNA, RNAi and DNA, said DNA inclusive of cDNA and genomic DNA. A nucleic acid may be native or recombinant and may comprise one or more artificial nucleotides, e.g., nucleotides not normally found in nature. Nucleic acids may include modified purines (for example, inosine, methylinosine, and methyladenosine) and modified pyrimidines (thiouridine and methylcytosine).

The terms “mRNA”, “RNA” and “transcript” are used interchangeably when referring to a transcribed copy of a transcribable nucleic acid.

A “polynucleotide” is a nucleic acid having eighty (80) or more contiguous nucleotides, while an “oligonucleotide” has less than eighty (80) contiguous nucleotides.

A “probe” may be a single or double-stranded oligonucleotide or polynucleotide, suitably labeled for the purpose of detecting complementary sequences in Northern blotting, Southern blotting or microarray analysis, for example.

A “primer” is usually a single-stranded oligonucleotide, preferably having 20-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™

### GmNF Receptor Proteins

In one aspect, the invention provides a soybean nodulation factor (NF) receptor protein.

In particular embodiments, the GmNF receptor protein is selected from the group consisting of a GmNFR1α protein, a GmNFR1β protein, GmNFR5α protein and a GmNFR5β protein.

Although not wishing to be bound by any particular theory, it is proposed that one or more of these proteins may be a component of a high-affinity receptor for the NF ligand.

Accordingly, in another aspect the invention provides an isolated nodulation factor receptor complex comprising at least one GmNF receptor protein selected from the group consisting of a GmNFR1α protein, a GmNFR1β protein, GmNFR5α protein and a GmNFR5β protein.

In one non-limiting embodiment, the invention contemplates a heterodimeric NF receptor complex comprising a GmNFR1 protein and a Gm NFR5 protein having a stoichiometry of 1:1.

The GmNFR1 protein may be a GmNFR1α protein or a GmNFR1β protein.

The GmNFR5 protein may be a GmNFR5α protein or a GmNFR5β protein.

By “protein” is also meant an amino acid polymer, comprising natural and/or non-natural amino acids, including L- and D-isomeric forms as are well understood in the art.

A “peptide” is a protein having no more than fifty (50) contiguous amino acids.

A “polypeptide” is a protein having more than fifty (50) contiguous amino acids.

In one embodiment, a protein “fragment” includes an amino acid sequence which constitutes less than 100%, but at least 20%, preferably at least 30%, more preferably at least 80% or even more preferably at least 90%, 95%, 96%, 97%, 98%, or 99% of a GmNF receptor protein.

The protein fragment may also be a “biologically active fragment” which retains biological activity of said protein.

The biologically active fragment of GmNFR1α or GmNFR1α protein preferably has greater than 10%, preferably greater than 20%, more preferably greater than 50% and even more preferably greater than 75%, 80%, 85%, 90%, 95%, 96%, 97%, 98%, or 99% of the biological activity of the entire protein.

Non-limiting examples of biological activities include NF ligand binding, protein kinase activity and/or an ability to associate with other GmNF receptor subunits to form a GmNF receptor complex.

Accordingly, GmNFR protein fragments may be in the form of isolated protein domains such as an extracellular domain, a LysM domain, a transmembrane domain, an intracellular domain and/or a protein kinase domain.

Another example of a biologically-active fragment is an N-terminal signal peptide of GmNFR1α protein as shown in FIG. 3B.

Other protein fragments contemplated by the present invention are encoded by one or more GmNFR gene exons.

In another embodiment, a “fragment” is a small peptide, for example of at least 6, preferably at least 10 and more preferably 15, 20, or 25 amino acids in length. Larger fragments comprising more than one peptide are also contemplated, and may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard, which is included in a publication entitled “Synthetic Vaccines” edited by

Nicholson and published by Blackwell Scientific Publications. Alternatively, peptides can be produced by digestion of a protein of the invention with suitable proteinases. The digested fragments can be purified by, for example, by high performance liquid chromatographic (HPLC) techniques.

As used herein, a “variant” protein is a GmNF receptor protein of the invention in which one or more amino acids have been deleted or substituted by different amino acids.

Variants include naturally occurring (e.g., allelic) variants, orthologs (i.e., from species other than Glycine max) and synthetic variants, such as produced in vitro using mutagenesis techniques.

Preferably, orthologs and paralogs are obtainable from plants such as peanut, bean, clovers, tomato, maize, rice, wheat, and the model crucifer Arabidopsis.

Variants may retain the biological activity of a corresponding wild type protein (e.g. allelic variants, paralogs and orthologs) or may lack, or have a substantially reduced, biological activity compared to a corresponding wild type protein.

In one particular embodiment, a GmNFR1α protein variant arises from a mis-sense mutant, which in exon 5 of GmNFR1α through a T deletion (1986Δ of the coding sequence) leads to a reading frame shift and protein termination within 5 amino acids. The encoded mutant protein would constitute a fragment lacking the entire protein kinase domain and presumably any biological activity.

In another particular embodiment, a GmNFR1α protein variant arises from a mutation in exon 4 by an A deletion (a769Δ) of GmNFR1α leading to protein termination within 51 amino acids. The encoded mutant protein would constitute a fragment lacking the entire protein kinase domain and presumably any biological activity.

In one particular embodiment, a GmNFR1β protein variant arises from a SNP in exon 10 that leads to a nonsense mutation at Q513.

In another particular embodiment, GmNFR1β protein variants are encoded by GmNFR1β gene splice variants such as set forth in FIGS. 13-15.

As will be appreciated from the foregoing, GmNFR protein variants may also be fragments of GmNFR proteins that may act to block, inhibit or otherwise affect GmNFR complex formation.

In other embodiments, variants include proteins having at least 75%, 80%, 85%, 90% or 95%, 96%, 97%, 98%, or 99% amino acid sequence identity to a GmNF receptor protein.

Terms used herein to describe sequence relationships between respective nucleic acids and proteins include “comparison window”, “sequence identity”, “percentage of sequence identity”, and “substantial identity”. Because respective nucleic acids/proteins may each comprise (1) only one or more portions of a complete nucleic acid/protein sequence that are shared by the nucleic acids/polypeptides, and (2) one or more portions which are divergent between the nucleic acids/proteins, sequence comparisons are typically performed by comparing sequences over a “comparison window” to identify and compare local regions of sequence similarity. A “comparison window” refers to a conceptual segment of typically at least 6, 8, 10, or 12 contiguous residues that is compared to a reference sequence. The comparison window may comprise additions or deletions (i.e., gaps) of about 20% or less as compared to the reference sequence (which does not comprise additions or deletions) for optimal alignment of the respective sequences. Optimal alignment of sequences for aligning a comparison window may be conducted by computerised implementations of algorithms (for example ECLUSTALW and BESTFIT provided by WebAngis GCG, 2D Angis, GCG, and GeneDoc programs, incorporated herein by reference) or by inspection and the best alignment (i.e., resulting in the highest percentage similarity or identity over the comparison window) generated by any of the various methods selected.

The ECLUSTALW program can be used to align multiple sequences. This program calculates a multiple alignment of nucleotide or amino acid sequences according to a method by Thompson, J. D., Higgins, D. G. and Gibson, T. J. (1994). This is part of the original ClustalW distribution, modified for inclusion in EGCG. The BESTFIT program aligns forward and reverse sequences and sequence repeats. This program makes an optimal alignment of a best segment of similarity between two sequences. Optimal alignments are determined by inserting gaps to maximize the number of matches using the local homology algorithm of Smith and Waterman. ECLUSTALW and BESTFIT alignment packages are offered in WebANGIS GCG (The Australian Genomic Information Centre, Building JO3, The University of Sydney, N.S.W 2006, Australia).

Reference also may be made to the BLAST family of programs as for example disclosed by Altschul et al., 1997, Nucl. Acids Res. 25, 3389, which is incorporated herein by reference.

A detailed discussion of sequence analysis can be found in Chapter 19.3 of Ausubel et al, supra.

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, U) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software Engineering Co., Ltd., South San Francisco, Calif., USA).

With regard to protein variants, these can be created by mutagenizing a protein or an encoding nucleic acid, such as by random mutagenesis or site-directed mutagenesis. Examples of nucleic acid mutagenesis methods are provided in Chapter 9 of CURRENT PROTOCOLS IN MOLECULAR BIOLOGY, Ausubel et al., supra which is incorporated herein by reference.

It will be appreciated by the skilled person that site-directed mutagenesis is best performed where knowledge of the amino acid residues that contribute to biological activity is available.

In cases where this information is not available, or can only be inferred by molecular modeling approximations, for example, random mutagenesis is contemplated. Random mutagenesis methods include chemical modification of proteins by hydroxylamine (Ruan et al., 1997, Gene 188, 35), incorporation of dNTP analogs into nucleic acids (Zaccolo et al., 1996, J. Mol. Biol. 255, 589) and PCR-based random mutagenesis such as described in Stemmer, 1994, Proc. Natl. Acad. Sci. USA 91, 10747 or Shafikhani et al., 1997, Biotechniques 23, 304, each of which references is incorporated herein. It is also noted that PCR-based random mutagenesis kits are commercially available, such as the Diversify™ kit (Clontech).

Mutagenesis may also be induced by chemical means, such as ethyl methane sulphonate (EMS) and/or irradiation means, such as fast neutron irradiation of seeds as known in the art and in particular relation to soybean (Carroll et al, 1985, Proc. Natl. Acad. Sci. USA 82, 4162; Carroll et al, 1985, Plant Physiol. 78, 34; Men et al., 2002, Genome Letters 3, 147).

As used herein, “derivative” proteins are proteins of the invention that have been altered, for example by conjugation or complexing with other chemical moieties or by post-translational modification techniques as would be understood in the art. Such derivatives include amino acid deletions and/or additions to polypeptides of the invention, or variants thereof.

“Additions” of amino acids may include fusion of the peptide or polypeptides of the invention, or variants thereof, with other peptides or polypeptides. Particular examples of such peptides include amino (N) and carboxyl (C) terminal amino acids added for use as fusion partners or “tags”.

Well-known examples of fusion partners include hexahistidine (6×-HIS)-tag, N-Flag, Fc portion of human IgG, glutathione-S-transferase (GST) and maltose binding protein (MBP), which are particularly useful for isolation of the fusion polypeptide by affinity chromatography. For the purposes of fusion polypeptide purification by affinity chromatography, relevant matrices for affinity chromatography may include nickel-conjugated or cobalt-conjugated resins, fusion polypeptide specific antibodies, glutathione-conjugated resins, and amylose-conjugated resins respectively. Some matrices are available in “kit” form, such as the ProBond™ Purification System (Invitrogene Corp.) which incorporates a 6×-His fusion vector and purification using ProBond™ resin.

The fusion partners may also have protease cleavage sites, for example enterokinase (available from Invitrogen Corp. as EnterokinaseMax™), Factor Xₐ, or Thrombin, which allow the relevant protease to digest the fusion polypeptide of the invention and thereby liberate the recombinant polypeptide of the invention therefrom. The liberated polypeptide can then be isolated from the fusion partner by subsequent chromatographic separation.

Fusion partners may also include within their scope “epitope tags”, which are usually short peptide sequences for which a specific antibody is available.

Other derivatives contemplated by the invention include, chemical modification to side chains, incorporation of unnatural amino acids and/or their derivatives during peptide or polypeptide synthesis and the use of cross linkers and other methods which impose conformational constraints on the polypeptides, fragments and variants of the invention.

Non-limiting examples of side chain modifications contemplated by the present invention include chemical modifications of amino groups, carboxyl groups, guanidine groups of arginine residues, sulphydryl groups, tryptophan residues, tyrosine residues, and/or the imidazole ring of histidine residues, as are well understood in the art.

Non-limiting examples of incorporating unnatural amino acids and derivatives during peptide synthesis include, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine, and/or D-isomers of amino acids.

Recombinant GmNF receptor proteins may be conveniently expressed and purified by a person skilled in the art using commercially available kits, for example.

Recombinant proteins may be produced, as for example described in Sambrook, et al., MOLECULAR CLONING. A Laboratory Manual (Cold Spring Harbor Press, 1989), incorporated herein by reference, in particular Sections 16 and 17; CURRENT PROTOCOLS IN MOLECULAR BIOLOGY Eds. Ausubel et al., (John Wiley & Sons, Inc. 1995-1999), incorporated herein by reference, in particular Chapters 10 and 16; and CURRENT PROTOCOLS IN PROTEIN SCIENCE Eds. Coligan et al., (John Wiley & Sons, Inc. 1995-1999) which is incorporated by reference herein, in particular Chapters 1, 5, 6, and 7.

### Isolated GmNF Receptor Nucleic Acids, Promoters and Chimeric Genes

The invention provides isolated GmNF receptor genes and structural components thereof, such as protein coding regions or open reading frames (ORFs), promoters and promoter active fragments, exons, introns and their respective splice sequences, 5′ and 3′ untranslated sequences, although without limitation thereto.

In one particular embodiment, the invention provides an isolated GmNFR1α nucleic acid (SEQ ID NO:5), which comprises:
(i) a nucleotide sequence encoding a GmNFR1α protein (SEQ ID NO:9);
(ii) a promoter-active nucleotide sequence (SEQ ID NO:13); and
(iii) a 3′ untranslated sequence.

In another particular embodiment, the invention provides an isolated GmNFR1β nucleic acid (SEQ ID NO:6), which comprises:
(i) a nucleotide sequence encoding a GmNFR1β protein (SEQ ID NO:10);
(ii) a promoter-active nucleotide sequence (SEQ ID NO:14); and
(iii) a 3′ untranslated sequence.

In yet another particular embodiment, the invention provides an isolated GmNFR5α nucleic acid (SEQ ID NO:7), which comprises:
(i) a nucleotide sequence encoding a GmNFR5α protein (SEQ ID NO:11);
(ii) a promoter-active nucleotide sequence (SEQ ID NO:15); and
(iv) a 3′ untranslated sequence.

In still yet another particular embodiment, the invention provides an isolated GmNFR5β nucleic acid (SEQ ID NO:8), which comprises:
(i) a nucleotide sequence encoding a GmNFR5β protein (SEQ ID NO:12);
(ii) a promoter-active nucleotide sequence (SEQ ID NO:16); and
(iii) a 3′ untranslated sequence.

The isolated nucleic acids of the invention may be particularly advantageous when expressed in a genetically modified plant, to thereby enhance, improve or otherwise facilitate plant nodulation.

As will be described in more detail hereinafter, increased nodulation coupled with nitrogen gain (and potential yield) has been demonstrated after over-expression of the modulation receptor component GmNFR1α.

Alternatively, isolated nucleic acids may be expressed as RNAi or anti-sense constructs to facilitate down-regulation of GmNFR1α, GmNFR1β, GmNFR5α, and/or GmNFR5β expression in plants.

The invention also contemplates fragments of isolated nucleic acids of the invention such as may be useful for recombinant protein expression or as probes, primers and the like.

A particular example of a nucleic acid fragment is a protein-coding or open reading frame sequence set forth in SEQ ID NO:5, SEQ ID NO:6, SEQ ID NO:7, SEQ ID NO:8, which respectively encode SEQ ID NO:1, SEQ ID NO:2, SEQ ID NO:3, and SEQ ID NO:4.

Another particular example is a 3′UTR fragment which may be useful for diagnostics and in RNAi methods.

Yet another particular example of a nucleic acid fragment is an exon or intron fragment of a GmNFR nucleic acid.

Still yet another particular example of a nucleic acid fragment is a “promoter” or “promoter-active fragment” of a GmNFR nucleic acid.

In particular embodiments, said promoter or promoter-active fragment comprises a nucleotide sequence present or contained in the 5′UTR sequences set forth in SEQ ID NOS: 13-16.

A promoter-active fragment comprises a nucleotide sequence, typically 5′ of a protein coding sequence, which is capable of initiating, directing, controlling or otherwise facilitating RNA transcription of the protein coding sequence.

This promoter activity may be manifested by the transcription of an autologous protein coding sequence (e.g., a GmNF receptor protein) or a by the transcription of heterologous protein coding sequence, such as in the context of a chimeric gene construct.

Thus, promoters of the invention may be particularly useful for facilitating expression of GmNF receptor protein, or heterologous sequences of interest (e.g., bio-pharmaceutical proteins) in plants, including but not limited to, soybean.

Heterologous sequences may be any sequence of interest inclusive of sequences that facilitate plant disease resistance, drought resistance, pest resistance, salt tolerance or other desirable traits, production of bio-pharmaceutical proteins and/or enzymes that direct or otherwise enable production of bioplastics or other biopolymers, although without limitation thereto.

The invention also contemplates variant nucleic acids of the invention.

As used herein, the term “variant”, in relation to an isolated nucleic acid, includes naturally-occurring allelic variants.

For example, the invention provides a GmNFR1α nucleic acid variant in the form of a mis-sense mutant, which in exon 5 of GmNFR1α through a T deletion (T986Δ of the coding sequence) leads to a reading frame shift and protein termination within 5 amino acids; a GmNFR1α nucleic acid variant mutated in exon 4 by an A deletion (A769Δ) of GmNFR1α leading to protein termination within 51 amino acids; and an SNP in exon 10 that leads to a nonsense mutation at Q513* in a GmNFR1β protein.

Other examples of nucleic acid variants include splice variants of a GmNFR1β nucleic acid such as:
(i) an extra CAG sequence at the exon 3-4 junction presumably derived from the 3′ end of intron 3 (FIG. 13);
(ii) complete loss of exon 5 (which created an earlier stop codon (TGA) in exon 7; FIG. 14); and
(iii) the complete loss of exon 8 together with a CAG exon 3-4 addition (which created a termination codon (TGA) in exon 9; FIG. 15).

Variants also include nucleic acids that have been mutagenized or otherwise altered so as to encode a protein having the same amino acid sequence (e.g., through degeneracy), or a modified amino acid sequence.

In the context of promoters, a “variant” nucleic acid may be mutagenized or otherwise altered to have little or no effect upon promoter activity, for example in cases where more convenient restriction endonuclease cleavage and/or recognition sites are introduced without substantially affecting the encoded protein or promoter activity. Other nucleotide sequence alterations may be introduced so as to modify promoter activity. These alterations may include deletion, substitution or addition of one or more nucleotides in a promoter. The alteration may either increase or decrease activity as required. In this regard, nucleic acid mutagenesis may be performed in a random fashion or by site-directed mutagenesis in a more “rational” manner. Standard mutagenesis techniques are well known in the art, and examples are provided in Chapter 9 of CURRENT PROTOCOLS IN MOLECULAR BIOLOGY, Eds. Ausubel et al. (John Wiley & Sons NY, 1995), which is incorporated herein by reference. Mutagenesis also includes mutagenesis using chemical and/or irradiation methods such as EMS and fast neutron mutagenesis of plant seeds.

In another embodiment, nucleic acid variant are nucleic acids having one or more codon sequences altered by taking advantage of codon sequence redundancy.

A particular example of this embodiment is optimization of a nucleic acid sequence according to codon usage as is well known in the art. This can effectively “tailor” a nucleic acid for optimal expression in a particular organism, or cells thereof, where preferential codon usage has been established.

Nucleic acid variants also include within their scope “homologs”, “orthologs”, and “paralogs”.

Nucleic acid orthologs may encode orthologs of a GmNF receptor protein of the invention that may be isolated, derived or otherwise obtained from plants other than Glycine max.

Preferably, orthologs are obtainable from plants such as peanut, bean, clovers, tomato, maize, and the model crucifer Arabidopsis.

In another embodiment, nucleic acid homologs share at least 65%, preferably at least 70%, more preferably at least 80% or 85% and even more preferably 90%, 95%, 96%, 97%, 98%, or 99%, sequence identity with a GmNF receptor nucleic acid of the invention.

In yet another embodiment, nucleic acid homologs hybridize to nucleic acids of the invention under high stringency conditions.

“Hybridise and Hybridisation” is used herein to denote the pairing of at least partly complementary nucleotide sequences to produce a DNA-DNA, RNA-RNA, or DNA-RNA hybrid. Hybrid sequences comprising complementary nucleotide sequences occur through base-pairing.

Modified purines (for example, inosine, methylinosine, and methyladenosine) and modified pyrimidines (thiouridine and methylcytosine) may also engage in base pairing.

“Stringency” as used herein, refers to temperature and ionic strength conditions, and presence or absence of certain organic solvents and/or detergents during hybridisation. The higher the stringency, the higher will be the required level of complementarity between hybridizing nucleotide sequences.

“Stringent conditions” designates those conditions under which only nucleic acid having a high frequency of complementary bases will hybridize.

Reference herein to high stringency conditions include and encompasses:—

(i) from at least about 31% v/v to at least about 50% v/v formamide and from at least about 0.01 M to at least about 0.15 M NaCl for hybridisation at 42° C., and at least about 0.01 M to at least about 0.15 M salt for washing at 42° C.;

(ii) 1% BSA, 1 mM EDTA, 0.5 M NaHPO₄ (pH 7.2), 7% SDS for hybridization at 65° C.; and (a) 0.1×SSC, 0.1% SDS, or (b) 0.5% BSA, 1 mM EDTA, 40 mM NaHPO₄ (pH 7.2), 1% SDS for washing at a temperature in excess of 65° C. for about one hour; and

(iii) 0.2×SSC, 0.1% SDS for washing at or above 68° C. for about 20 minutes.

Notwithstanding the above, stringent conditions are well-known in the art, such as described in Chapters 2.9 and 2.10 of Ausubel et al., supra, which are herein incorporated by reference. A skilled addressee will also recognize that various factors can be manipulated to optimize the specificity of the hybridization. Optimization of the stringency of the final washes can serve to ensure a high degree of hybridization.

Typically, complementary nucleotide sequences are identified by blotting techniques that include a step whereby nucleotides are immobilized on a matrix (preferably a synthetic membrane such as nitrocellulose), a hybridization step, and a detection step.

In light of the foregoing, it will be appreciated that variants, homologs and orthologs may be isolated by means such as nucleic acid sequence amplification techniques, (including but not limited to PCR, strand displacement amplification, rolling circle amplification, helicase-dependent amplification and the like) and techniques which employ nucleic acid hybridization (e.g., plaque/colony hybridization).

### Genetic Constructs and GmNF Receptor Protein Expression

A “genetic construct” comprises a nucleic acid of the invention or a chimeric gene, together with one or more other elements that facilitate manipulation, propagation, homologous recombination and/or expression of said nucleic acid or chimeric gene.

In a preferred form, the genetic construct is an expression construct, which is suitable for the expression of a nucleic acid or a chimeric gene of the invention.

The expression construct may be particularly advantageous when expressed in a genetically modified plant, to enhance, improve or otherwise facilitate plant nodulation.

Alternatively, expression constructs may be RNAi or anti-sense constructs that facilitate down-regulation of GmNF receptor expression in plants.

Typically, an expression construct comprises one or more regulatory sequences present in an expression vector, operably linked or operably connected to the nucleic acid of the invention or the chimeric gene, to thereby assist, control or otherwise facilitate transcription and/or translation of the nucleic acid or the chimeric gene of the invention.

By “operably linked” or “operably connected” is meant that said regulatory nucleotide sequence(s) is/are positioned relative to the nucleic acid or chimeric gene of the invention to initiate, regulate, or otherwise control transcription and/or translation

Regulatory nucleotide sequences will generally be appropriate for the host cell used for expression. Numerous types of appropriate expression vectors and suitable regulatory sequences are known in the art for a variety of host cells.

Typically, said one or more regulatory nucleotide sequences may include, promoter sequences, leader or signal sequences, ribosomal binding sites, transcriptional start and termination sequences, translational start and termination sequences, and enhancer or activator sequences.

A host cell or organism for nucleic acid and/or protein expression may be prokaryotic or eukaryotic.

In embodiments where a GmNFR protein coding sequence is to be expressed in a bacterial cell (e.g., E. coli DH5α or BL21), such as for recombinant protein production, an inducible promoter may be utilized, such as the IPTG-inducible lacZ promoter.

Other regulatory elements that may assist recombinant protein expression in bacteria include bacterial origins of replication (e.g., as in plasmids pBR322, pUC19, and the ColE1 replicon, which function in many E. coli. strains) and bacterial selection marker genes (amp^{r}, tet^{r}, and kan^{r}, for example).

In embodiments where a chimeric gene is to be expressed in a plant cell a promoter-active fragment of a GmNFR nucleic acid may be used as a promoter to facilitate expression of a heterologous sequence.

In embodiments where a GmNFR protein is to be expressed in a plant cell, the promoter-active fragment of a corresponding GmNFR nucleic acid may effectively act as an autologous promoter.

In alternative embodiments where a GmNFR protein is to be expressed in a plant cell, the expression construct may alternatively comprise a heterologous promoter operable in a plant.

Non-limiting examples of suitable heterologous promoters include the CaMV35S promoter, Emu promoter (Last et al., 1991, Theor. Appl. Genet. 81, 581), or the maize ubiquitin promoter Ubi (Christensen & Quail, 1996, Transgenic Research 5, 213).

A preferred heterologous promoter is the CaMV35S promoter.

Usually, when transgenic expression of a protein is required, a correct orientation of the encoding nucleic acid transgene is in the sense or 5′ to 3′ direction relative to the promoter. However, where antisense expression is required, the transcribable nucleic acid is oriented 3′ to 5′. Both possibilities are contemplated by the expression construct of the present invention, and directional cloning for these purposes may be assisted by the presence of a polylinker.

An expression vector may further comprise viral and/or plant pathogen nucleotide sequences. A plant pathogen nucleic acid includes T-DNA plasmid, modified (including for example a recombinant nucleic acid) or otherwise, from Agrobacterium.

The expression vector may further comprise a selectable marker nucleic acid to allow the selection of transformed cells.

In embodiments relating to expression in plants, suitable selection markers include, but are not limited to: neomycin phosphotransferase II, which confers kanamycin and geneticin/G418 resistance (nptII; Raynaerts et al., In: Plant Molecular Biology Manual A9:1-16, Gelvin & Schilperoort, Eds. (Kluwer, Dordrecht, 1988); bialophos/phosphinothricin resistance (bar; Thompson et al., 1987, EMBO J. 6, 1589); streptomycin resistance (aadA; Jones et al., 1987, Mol. Gen. Genet. 210, 86); paromomycin resistance (Mauro et al., 1995, Plant Sci. 112, 97); β-glucuronidase (gus; Vancanneyt et al., 1990, Mol. Gen. Genet. 220, 245); and hygromycin resistance (hmr or hpt; Waldron et al., 1985, Plant Mol. Biol. 5, 103; Perl et al., 1996, Nature Biotechnol. 14, 624).

Selection markers such as described above may facilitate selection of transformed plant cells or tissue by addition of an appropriate selection agent post-transformation, or by allowing detection of plant tissue which expresses the selection marker by an appropriate assay. In that regard, a reporter gene such as gfp, nptII, luc, or gusA may function as a selection marker.

Positive selection is also contemplated such as by the phosphomannine isomerase (PMI) system described by Wang et al., 2000, Plant Cell Rep. 19, 654 and Wright et al., 2001, Plant Cell Rep. 20, 429 or by the system described by Endo et al., 2001, Plant Cell Rep. 20, 60, for example.

The expression construct of the present invention may also comprise other gene regulatory elements, such as a 3′ non-translated sequence. A 3′ non-translated sequence refers to that portion of a gene that contains a polyadenylation signal and any other regulatory signals capable of effecting mRNA processing or gene expression. The polyadenylation signal is characterized by effecting the addition of polyadenylic acid tracts to the 3′ end of the mRNA precursor. Polyadenylation signals are commonly recognized by the presence of homology to the canonical form 5′ AATAAA-3′, although variations are not uncommon.

The 3′ non-translated regulatory DNA sequence preferably includes from about 300 to 1,000 nucleotide base pairs and contains plant transcriptional and translational termination sequences. Examples of suitable 3′ non-translated sequences are the 3′ transcribed non-translated regions containing a polyadenylation signal from the nopaline synthase (nos) gene of Agrobacterium tumefaciens (Bevan et al., 1983, Nucl. Acid Res., 11, 369) and the terminator for the T7 transcript from the octopine synthase (ocs) gene of Agrobacterium tumefaciens.

Tanscriptional enhancer elements include elements from the CaMV 35S promoter and octopine synthase (ocs) genes, as, for example, described in U.S. Pat. No. 5,290,924, which is incorporated herein by reference. It is proposed that the use of an enhancer element such as the ocs element, and particularly multiple copies of the element, may act to increase the level of transcription from adjacent promoters when applied in the context of plant transformation.

Additionally, targeting sequences may be employed to target a protein product of the transcribable nucleic acid to an intracellular compartment within plant cells or to the extracellular environment. For example, a DNA sequence encoding a transit or signal peptide sequence may be operably linked to a sequence encoding a desired protein such that, when translated, the transit or signal peptide can transport the protein to a particular intracellular or extracellular destination, respectively, and can then be post-translationally removed. Transit or signal peptides act by facilitating the transport of proteins through intracellular membranes, e.g., vacuole, vesicle, plastid, and mitochondrial membranes, whereas signal peptides direct proteins through the extracellular membrane. For example, the transit or signal peptide can direct a desired protein to a particular organelle such as a plastid (e.g., a chloroplast), rather than to the cytoplasm. Thus, the expression construct can further comprise a plastid transit peptide encoding DNA sequence operably linked between a promoter region or promoter variant according to the invention and transcribable nucleic acid. For example, reference may be made to Heijne et al., 1989, Eur. J. Biochem. 180, 535, and Keegstra et al., 1989, Ann. Rev. Plant Physiol. Plant Mol. Biol. 40, 471, which are incorporated herein by reference.

A genetic construct or vector may also include an element(s) that permits stable integration of the vector into the host cell genome or autonomous replication of the vector in the cell independent of the genome of the cell. The vector may be integrated into the host cell genome when introduced into a host cell. For integration, the vector may rely on the foreign or endogenous DNA sequence or any other element of the vector for stable integration of the vector into the genome by homologous recombination. Alternatively, the vector may contain additional nucleic acid sequences for directing integration by homologous recombination into the genome of the host cell. The additional nucleic acid sequences enable the vector to be integrated into the host cell genome at a precise location in the chromosome. To increase the likelihood of integration at a precise location, the integrational elements should preferably contain a sufficient number of nucleic acids, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences.

The expression construct, whether for expression in plant, bacterial or other host cells, may also include a fusion partner (typically provided by the expression vector) so that a recombinant GmNFR protein is expressed as a fusion protein with the fusion partner, as hereinbefore described. An advantage of fusion partners is that they assist identification and/or purification of the fusion protein. Identification and/or purification may include using a monoclonal antibody or substrate specific for the fusion partner.

### Plant Transformation and Genetically Modified Plants

Other aspects of the present invention relate to genetically-modified or “transgenic” plants, plant tissues, and/or plant cells, and a method of producing transgenic plants.

The identification and cloning of GmNF receptor genes opens up a possibility of beneficially manipulating plant nodulation and plant root systems. Plants, including crops, forests, pasture and garden plants, are completely dependent on a healthy root system for absorption of water and nutrients from soil. It is now possible that transgenic over-expression of one or more GmNF receptor genes (e.g., GmNFR1α in particular) may improve an ability of a plant to absorb water and nutrients from soil. Such transgenic plants may have increased water and nutrient absorption thereby improving crop yields.

Enhanced or increased nodulation (e.g., super- or hypernodulation) can increase nitrogen fixation. Transgenic plants made in accordance with the present invention may be engineered to increase nodulation and nitrogen fixation in legumes including soybean, Phaseolus beans, azukibeans, Faba beans, peas, peanuts, clovers, lentils, chickpea, pigeonpea, black eyed pea (cowpea), siratro, acacias, and non-legume crops like tomato, potato, cotton, canola, grapes, sorghum, wheat, rice, and maize, thereby decreasing a requirement for nitrogen fertilizers. Enhanced or increased nodulation may also be useful when using nodules as bio-factories to produce a desired compound, such as a bio-active compound or biologically active protein for use in a pharmaceutical composition. Increasing the number and/or frequency of nodules may improve yield and ease of harvesting of the bio-active compound that may be recombinantly expressed or endogenous to the nodule and/or symbiotic organism of the nodule.

Non-limiting examples of bio-active compounds include phytoestrogens, isoflavones, flavones and iron complexing molecules.

Alternatively, down-regulation of GmNF receptor expression (such as by RNAi or antisense expression) in plants may be advantageous where reduced nodulation or nitrogen fixation is required.

It will be appreciated that “relatively” increased or reduced nodulation and/or nitrogen fixation is typically determined by comparison of nodulation and/or nitrogen fixation in a plant without genetic modification, preferably of the same plant species.

In one embodiment, the method of producing a transgenic plant, plant cell or tissue, includes the steps of:
(i) transforming a plant cell or tissue with a genetic construct comprising an isolated GmNFR nucleic acid; and
(ii) selectively propagating a transgenic plant from the plant cell or tissue transformed in step (i).

Suitably, the plant cell or tissue used at step (i) may be a leaf disk, callus, meristem, hypocotyls, root, leaf spindle or whorl, leaf blade, stem, shoot, petiole, axillary bud, shoot apex, internode, cotyledonary-node, flower stalk, or inflorescence tissue.

Preferably, the plant tissue is a leaf or part thereof, including a leaf disk, hypocotyl, or cotyledonary-node.

The plant cell or tissue may be obtained from any plant species including monocotyledon, dicotyledon, ferns, and gymnosperms, such as conifers, without being limited thereto.

Preferably, the plant is a dicotyledon or a monocotyledon, inclusive of crop plants such as legumes and cereals.

The plant may be, for example, wheat, maize, rice, tobacco, Arabidopsis, legumes, such as soybean, Glycine max, Glycine soja L., pea, cowpea, Phaseolus bean, broadbean, lentils, chickpea, peanuts, acacia trees, clovers, siratro, alfalfa, Lotus japonicus, Lotus corniculatus, or Medicago truncatula.

Persons skilled in the art will be aware that a variety of transformation methods are applicable to the method of the invention, such as Agrobacterium tumefaciens-mediated (Gartland & Davey, 1995, Agrobacterium Protocols (Humana Press Inc. NJ USA); U.S. Pat. No. 6,037,522; WO99/36637), microprojectile bombardment (Franks & Birch, 1991, Aust. J. Plant. Physiol., 18, 471; Bower et al., 1996, Molecular Breeding, 2, 239; Nutt et al., 1999, Proc. Aust. Soc. Sugar Cane Technol. 21, 171), liposome-mediated (Ahokas et al., 1987, Heriditas 106, 129), laser-mediated (Guo et al., 1995, Physiologia Plantarum 93, 19), silicon carbide or tungsten whiskers (U.S. Pat. No. 5,302,523; Kaeppler et al., 1992, Theor. Appl. Genet. 84, 560), virus-mediated (Brisson et al., 1987, Nature 310, 511), polyethylene-glycol-mediated (Paszkowski et al., 1984, EMBO J. 3, 2717), as well as transformation by microinjection (Neuhaus et al., 1987, Theor. Appl. Genet. 75, 30) and electroporation of protoplasts (Fromm et al., 1986, Nature 319, 791), all of which references are incorporated herein.

Agrobacterium-mediated transformation may utilize A. tumefaciens or A. rhizogenes.

As will be described in more detail hereinafter, expression of GmNFR1α protein was achieved in plants by a method employing Agrobacterium rhizogenes cucumapine strain K599 carrying the GmNFR1α cDNA driven by either its own 3.5 kb native promoter or the constitutive 35S CaMV promoter in binary vector pCAMBIA1305.1.

It is also contemplated that co-expression of GmNFR1α protein and GmNFR5α protein may further enhance, improve, enhance and/or otherwise facilitate nodulation and/or nitrogen fixation.

Preferably, selective propagation at step (ii) is performed in a selection medium comprising geneticin as selection agent.

In one embodiment, the expression construct may further comprise a selection marker nucleic acid as hereinbefore described.

In another embodiment, a separate selection construct may be included at step (i), which comprises a selection marker nucleic acid.

The transformed plant material may be cultured in shoot induction medium followed by shoot elongation media as is well known in the art. Shoots may be cut and inserted into root induction media to induce root formation as is known in the art.

It will be appreciated that as discussed hereinbefore, there are a number of different selection agents useful according to the invention, the choice of selection agent being determined by the selection marker nucleic acid used in the expression construct or provided by a separate selection construct.

### Detection of Transgene Expression

The “transgenic” status of genetically-modified plants of the invention may be ascertained by measuring expression of a GmNF receptor protein or nucleic acid.

In one embodiment, transgene expression can be detected by an antibody specific for a GmNF receptor protein:
(i) in an ELISA such as described in Chapter 11.2 of CURRENT PROTOCOLS IN MOLECULAR BIOLOGY Eds. Ausubel et al. (John Wiley & Sons Inc. NY, 1995), which is herein incorporated by reference; or
(ii) by Western blotting and/or immunoprecipitation such as described in Chapter 12 of CURRENT PROTOCOLS IN PROTEIN SCIENCE Eds. Coligan et al. (John Wiley & Sons Inc. NY, 1997), which is herein incorporated by reference.

Protein-based techniques such as mentioned above may also be found in Chapter 4.2 of PLANT MOLECULAR BIOLOGY: A Laboratory Manual, supra, which is herein incorporated by reference.

It will also be appreciated that transgenic plants of the invention may be screened for the presence of mRNA corresponding to a transcribable nucleic acid and/or a selection marker nucleic acid. This may be performed by RT-PCR (including quantitative RT-PCR), Northern hybridization, and/or microarray analysis. Southern hybridization and/or PCR may be employed to detect DNA (the GmNFR1α or fi promoters, GmNFR1α or β mutants, transcribable nucleic acid and/or selection marker) in the transgenic plant genome using primers such as described herein in the Examples.

For examples of RNA isolation and Northern hybridization methods, the skilled person is referred to Chapter 3 of PLANT MOLECULAR BIOLOGY: A Laboratory Manual, supra, which is herein incorporated by reference. Southern hybridization is described, for example, in Chapter 1 of PLANT MOLECULAR BIOLOGY: A Laboratory Manual, supra, which is herein incorporated by reference.

A selectable marker as described herein is typically used to increase the number of positive transformants before assaying for transgene expression. However, positive transformants identified by PCR and other high throughput type systems (e.g., microarrays) enable selection of transformants without use of a selectable marker due to a large number of samples that may be easily tested. It may be preferred to avoid use of selectable markers in transgenic plants because of environmental concerns in relation to perceived accidentally release of the selectable marker nucleic acid into the environment. Herbicide resistance markers, e.g., against BASTA, and antibiotic resistance markers, e.g., against ampicillin, are a few selectable markers that may be of concern. PCR may be performed on thousands of samples using primers specific for the transgene or part thereof, the amplified PCR product may be separate by gel electrophoresis, coated onto multi-well plates and/or dot blotting onto a membrane and hybridized with a suitable probe, for example probes described herein including radioactive and fluorescent probes to identify the transformant.

Anti-GmNF receptor protein antibodies of the invention may be polyclonal or monoclonal. Well-known protocols applicable to antibody production, purification and use may be found, for example, in Chapter 2 of Coligan et al., CURRENT PROTOCOLS IN IMMUNOLOGY (John Wiley & Sons NY, 1991-1994) and Harlow, E. & Lane, D. Antibodies: A Laboratory Manual, Cold Spring Harbor, Cold Spring Harbor Laboratory, 1988, which are both herein incorporated by reference.

Generally, antibodies of the invention bind to or conjugate with a polypeptide, fragment, variant or derivative of the invention. For example, the antibodies may comprise polyclonal antibodies. Such antibodies may be prepared for example by injecting a polypeptide, fragment, variant or derivative of the invention into a production species, which may include mice, rabbits or goats, to obtain polyclonal antisera. Methods of producing polyclonal antibodies are well known to those skilled in the art. Exemplary protocols that may be used are described for example in Coligan et al., CURRENT PROTOCOLS IN IMMUNOLOGY, supra, and in Harlow & Lane, 1988, supra.

In lieu of the polyclonal antisera obtained in the production species, monoclonal antibodies may be produced using the standard method as for example, described in an article by Köhler & Milstein, 1975, Nature 256, 495, which is herein incorporated by reference, or by more recent modifications thereof as for example, described in Coligan et al., CURRENT PROTOCOLS IN IMMUNOLOGY, supra, by immortalizing spleen or other antibody producing cells derived from a production species which has been inoculated with one or more of the polypeptides, fragments, variants, or derivatives of the invention.

The invention also includes within its scope antibodies that comprise Fc or Fab fragments of the polyclonal or monoclonal antibodies referred to above. Alternatively, the antibodies may comprise single chain Fv antibodies (scFvs) against the peptides of the invention. Such scFvs may be prepared, for example, in accordance with the methods described respectively in U.S. Pat. No. 5,091,513, European Patent 239,400 or the article by Winter & Milstein, 1991, Nature 349, 293, which are incorporated herein by reference.

In order that the invention may be readily understood and put into practical effect, particular preferred embodiments will now be described by way of the following non-limiting examples.

### EXAMPLES

### Example 1

### GmNFR1α and GmNFR1β

### Materials and Methods

Hairy Root Transformation.

For hairy root complementation, the GmNFR1α cDNA driven by either its own (3.4 kb) or the CaMV 35S promoter were constructed in the binary vector pCAMBIA1305.1. The constructs were introduced into A. rhizogenes strain K599 by electroporation. For the transformation experiments bacteria grown for overnight at 28° C. were collected from four LB plates containing 50 μg/mL kanamycin and suspended in 5 mL of sterile water.

Soybean seeds were surface-sterilized by soaking in 0.5% (v/v) hydrogen peroxide in 70% ethanol for 5 min and then rinsed 10 times in sterile distilled water.

Sterilized seeds were germinated in sterile vermiculite under 16 h light at 28° C.

Five days old seedlings with unfolded cotyledons were inoculated by piercing three times the hypocotyls through the vascular bundles with a needle and delivering 3-4 drops of inoculum into the wound. Inoculated plants were watered with B&D solution (Broughton & Dilworth, 1971, Biochem. J. 125, 1075) containing 2 mM KNO₃.

Hairy-roots appeared from the wounded nodal region about 2 weeks after inoculation. One week later the primary roots were removed about 2 cm below the cotyledonary node prior to transferring the plants into new pots filled with vermiculite. Five days later, the plants were inoculated with 3 mL of 10⁷ cells of Bradyrhizobium japonicum CB1809 and nodulation were scored 3-5 weeks after the inoculation.

Nitrogen Determination.

Composite plants were grown in nitrogen-free conditions and inoculated with CB1809. Five weeks after inoculation, plants were sacrificed and ground to a powder prior to elemental analysis at the Natural Resources, Agriculture and Veterinary Science (NRAVS) University of Queensland facility.

Nucleic Acid Isolation.

The genomic DNA from soybean plants was isolated with the help of the DNAeasy Plant Mini Kit of Qiagen. For the purification of the plasmid and BAC clones the QIAprep Spin Miniprep Kit (Qiagen) and the PSI Clone BigBAC DNA isolation kit (Princeton Separations), respectively, were used according to the instructions of the suppliers.

For Reverse Transcription (RT) PCR total RNA was isolated from root and hypocotyl tissues of uninoculated or inoculated plants followed by DNaseI treatment using the NucleoSpin RNA Plant kit (Macherey-Nagel). For quantitative real time PCR, total RNA was extracted from inoculated hairy root of nod49 and Bragg transformed either with empty vector, native promoter+GmNFR1α or β, or 35S promoter+GmNFR1α or β using similar kit as for Reverse Transcriptase PCR. Each RNA preparation was reverse transcribed with oligo dT, i.e., specifically TTTTTTTTTTTTTTTTTTTTV [SEQ ID NO:86], wherein V=A, G, or C, and Superscript III (Invitrogen Australia Pty. Ltd.).

Primers.

Primers for amplifying GmNFR1 probes and testing BAC clones are identified in Table 5.

Primers for RT-PCR are identified as SEQ ID NOS: in Table 5.

Primers used for sequencing BAC clones are identified in Table 5.

PCR Methods.

DNA fragments were amplified by Tag (GIBCO BRL) or Pfu (FERMENTAS) DNA polymerases in a PTC-200™ Programmable Thermal Controller (MJ Research, Inc.) using specific primers shown in Table 5 and 150 ng of soybean genomic DNA or 4.0 μl of plant cDNA or, 25 ng of BAC DNA as template. Samples were heated to 95° C. for 2 min, followed by 35 cycles of denaturation at 94° C. for 60 seconds, annealing for 30 seconds, elongation at 72° C. for 60-120 seconds, and a final extension at 72° C. for 5 minutes. Amplified products were separated by electrophoresis in 1% or 2% agarose gels in 1×TAE buffer and were detected by fluorescence under UV light (302 nm).

Quantitative Real-Time PCR.

cDNA was subjected to real-time PCR with specific primer pairs (Table 5) and 1×SYBR Green according to the manufacturer's instruction (PE Applied Biosystems) using an ABIM prism thermocycler. Real time PCR was carried out in a total volume of 25 μL and contained 5 μL (˜200 ng) cDNA, 0.2 μM of each primer pair and 1×SYBR green PCR master mix (PE Applied Biosystem). The reaction mixture was heated at 95° C. for 10 min and then subjected to 45 PCR cycles of 95° C. for 15 s and 60° C. for 60 s, the resulting fluorescent being monitored. Heat dissociation curves were performed at 95° C. for 2 min, 60° C. for 15 s, and 95° C. for 15 s.

Sequencing of BAC Clones.

Sequencing reaction was performed in the same PCR engine using DNA isolated from BAC clones 55N1 and 54B21. Sequencing mixture consisted of 4.0 μl of 200 ng/mL BAC DNA, 1.0 μl of Ready reaction premix (MBI Fermentas), 3.0 μl of BigDye sequencing buffer, 2.0 μl of 2 μM primer (Table 5), and 5 μL of distilled water. Samples were heated to 94° C. for 5 min, followed by 40 cycles at 96° C. (30 s), 50° C. (15 s) and 60° C. (240 s).

Statistical Methods.

Analysis of variance (ANOVA) was used to identify if there were significant effects of the treatments (empty vector, native promoter, and 35S promoter) on the variables: nodule number/plant, nitrogen percentage, and total nitrogen. Where significant effects were found, the Least Significant Difference Separation Procedure was used to separate the differences.

### Results

To aid their elucidation, allelic non-nodulation (nod) mutants nod49 (Carroll et al., 1986, Plant Sci 47, 109; Mathews et al., 1989, J. Hered. 80, 357) and rj1 (Weber 1966, Agron. J. 46, 28) were isolated from either EMS-mutagenized or natural populations (FIG. 1B). Non-nodulation and associated nitrogen deficiency of such mutants, reminiscent of nodulation failures produced by environmental stresses, lead to growth retardation and subsequent yield losses in the absence of mineralized nitrogen (FIG. 1A; Carroll et al., supra).

Nodule development is tightly controlled by the inoculation process itself as well as a systemic feedback process called ‘Autoregulation of Nodulation’ (AON), which, if mutated leads to hyper- or supernodulation (Kinkema et al., 2006, Funct. Plant Biol., 31, 707; Searle et al., 2003, Science 299, 108; Carroll et al., 1985, Proc. Natl. Acad. Sci. USA 82, 4162; Wopereis et al., 2000, Plant J., 23, 97; Krusell et al., 2002, Nature 420, 422; Nishimura et al., 2002, Nature 420, 426; Sagan et al., Plant Sci. 1996, 117, 167; Schnabel et al., 2005, Plant Physiol. 58, 809). AON mutants are characterised by increased nodule number, earlier nodulation onset, partial insensitivity to the inhibitory effects of nitrate and acid soils, decreased main root growth, and an increased proportion of the primary root with nodules (the so-called ‘nodulation interval’). Penetrance of symbiotic effects of AON receptor kinase mutants varies among species, so that Ljhar1 mutants have severe root retardation while soybean GmNARK mutants are predominantly affected in nodule and not root development.

The mutation of nod49, chemically induced in soybean cultivar Bragg segregates as a single Mendelian recessive allele; it is allelic to the naturally occurring rj1 mutation. Its phenotype includes: (i) root control of nodulation block (Delves et al., 1986, Plant Physiol. 82, 588), (ii) normal root exudation for Bradyrhizobium nod gene induction (Sutherland et al., 1990, Mol. Plant Microbe Interact. 3, 122; Mathews et al., 1989, Mol. Plant Microbe Interact. 2, 283), (iii) lack of root hair deformation (Had; FIG. 1E), curling (Hac) and infection thread growth (Inf) (Mathews et al., 1990, Theor. Appl. Genet. 79, 125), and (iv) wild-type ability of mycorrhizal associations (Myc⁺; FIGS. 1C,D). Histology of nod49, rj1 and wild type Bragg (Mathews et al., 1990, supra) showed that despite the absence of infection-related events (e.g., Had, Hac, and Inf), the nod mutants developed subepidermal CCDs; FIG. 1F) that failed to develop further. In wild-type soybean such ‘pseudo-infections’ if associated with a successful root hair infection event, develop into ‘actual infections’ (Mathews et al., 1987, Plant Physiol. 131, 349; FIG. 1G) and eventually nodules (Mathews et al., 1990, supra). Significantly, mutants nod49 and rj1, inoculated with ultra-high B. japonicum titers (greater than 10⁸ cells per mL), occasionally formed 1 to 5 fully functional nodules per plant through a wild-type Had/Hac/Inf pathway (Mathews et al., 1990, supra; Mathews et al., 1987, supra; Mathews et al., 1989, Protoplasma 150, 40). This biological result already suggested that the nod49/rj1 mutants are altered at an early perception stage.

Many symbiosis-controlling genes of soybean have been mapped (e.g., Landau-Ellis et al., 1991, Mol. Gen. Genet. 228, 221) but only one, GmNARK has been map-based cloned (encoding the nodule autoregulation receptor kinase; Searle et al., 2003, supra; Carroll et al., 1985, supra; Wopereis et al., 2000, supra; Krusell et al., 2002, supra; Nishimura et al., 2002, supra; Sagan et al., 1996, supra; Schnabel et al., 2005, supra). Mutant nod49 was crossed with Glycine soja CPI 100070 (a polymorphic wild-type relative), and analysis of resultant F₂ plants, segregating at a 3:1 wild type-to-mutant ratio, positioned the nod49 locus within 3 cM of SSR marker Satt459 on Molecular Linkage Group (MLG) D1B (FIG. 2A). Interrogation of several molecular linkage maps detected RFLP markers K411, A343, T270 and A135 in the vicinity of Satt459, but these were not mapped in the mapping population. As Satt459 was the only marker mapped directly to nod49, its map distance of 3 cM was too large for a ‘chromosome walk’.

Reflecting an ancestral duplication of the soybean genome (Song et al., 2004, Theor. Appl. Genet. 109, 122), the region around Satt459 is duplicated on MLG B2, maintaining the approximate map order and distances of several RFLP markers (FIG. 2A). Fortuitously the translated DNA sequence of the probes for two linked RFLP markers, namely K411-1 and A343-2, shared high amino acid identity with the C and N termini of LysM type receptor kinases. As mutations in genes coding for LysM type receptor kinases LjNFR1, LjNFR5, and MtNFP1 (and partially MtLYK3) resulted in a similar Nod⁻ Myc⁺phenotype (Radutoiu et al., 2003, Nature 425, 585; Madsen et al., 2003, Nature 425, 637; Limpens et al., 2003, Science 302, 630; Amor et al., 2003, Plant J. 34, 495), we progressed with a candidate gene approach involving allele sequencing, complementation, and over-expression analysis.

PCR primers designed from the sequences of K411 and A343 and genomic DNA of Bragg as template were used to amplify a PCR product, which was cloned and its sequence proved to be collinear to LjNFR1, the NF receptor component gene of the model legume Lotus japonicus. This PCR product was then used to screen a Bacterial Artificial Chromosome (BAC) library of wild-type soybean variety PI437.654 (Tomkins et al., 1999, Plant Mol. Biol. 41, 25). Eight positively hybridizing BAC clones were characterized by fingerprinting following HindIII digestion (FIG. 2B). Three distinct HindIII digestion patterns were detected, one shown later to be a false positive (lane 5), one characterized by BAC54B21 (lane 3), the other by BAC55N1 (lane 6). Reflecting duplication found in the molecular map (c.f. FIG. 2A), this finding suggested the existence of two separate homeologous regions containing DNA sequences of the putative NFR1 receptor genes in the soybean genome.

Isolated BAC DNA from the two regions was used as template in PCR reactions to verify the presence of the probed sequence (FIG. 2C), and produced products of two sizes (referred to as α and β fragments), differing by 374 bp (FIG. 2B); Bragg genomic DNA amplified both α and β fragments. Sequencing of these products revealed two highly related DNA stretches similar to the LysM receptor kinase gene family. As RFLP markers K411 and A343 exist in two soybean linkage groups, the two regions defined by the BACs were assumed to represent these loci, and were considered to be good candidates for the location of the nod49/rj1 mutations.

It was necessary to discern which of these regions harbored the nod49/rj1 mutations, and to reveal the function, if any, of the duplicated region. The Nod⁻ trait in mutants nod49 and rj1 behaves as classical monogenic, recessive loss-of-function mutation with a leaky phenotype suggesting that the duplicated region lacks or could not fulfill the same symbiotic function. The regions of BAC54B21 and BAC55N1 related to the LysM receptor kinase were sequenced to reveal the entire gene sequences and the putative promoters of GmNFR1α (3.4 kb) and GmNFR1β(1.0 kb; accession number: DQ219806, DQ219809). Both genes share high level of identity with LjNFR1 in exon-intron structure and DNA sequence (FIG. 3A).

A soybean cDNA library derived from uninoculated root of Bragg was screened, then 3′ anchored clones with 100% homology to the ORFs defined in the genomic PCR products were extended by 5′RACE to give the full-length cDNAs of two related LysM receptor kinase genes with high homology (average 82% nucleotide identity) to LjNFR1. RT-PCR demonstrated that both genes are expressed in soybean root and hypocotyl tissue independent of the inoculation status with B. japonicum (FIG. 5). However, quantitative RT-PCR suggests that GmNFR1α mRNA levels are about 90 fold higher than those of GmNFR1β. Entire cDNA sequences for GmNFR1α and GmNFR1β are shown in FIG. 6 and FIG. 7, respectively. These sequences include 5′ UTR comprising a promoter region, a coding sequence and a 3′ UTR.

An alignment between the coding sequences of GmNFR1α, GmNFR1β, LjNFR1 and MTLYK3 is shown in FIG. 8.

An alignment between the promoters of GmNFR1α and GmNFR1β and the LjNFR1 promoter is shown in FIG. 9.

Exon sequences of both GmNFR1α and GmNFR1β are shown in FIG. 10 and FIG. 11.

Aligned amino acid sequences of GmNFR1α and GmNFR1β proteins are shown in FIG. 12.

Alternative splicing of GmNFR1β, but not GmNFR1α, was observed when sequencing full length cDNA clones. Radutoiu et al. (2) already observed the addition of two codons (GTA-ATG), presumably derived from the 5′ end of intron 4 at the 3′ end of exon 4 in an alternative transcript of LjNFR1. We observed the addition of an extra CAG sequence at the exon 3-4 junction presumably derived from the 3′ end of intron 3 (FIG. 13). We also detected other alternative transcripts with either (i) the complete loss of exon 5 (which created an earlier stop codon (TGA) in exon 7; FIG. 14), or (ii) the complete loss of exon 8 together with a CAG exon 3-4 addition (which created a termination codon (TGA) in exon 9; FIG. 15). GmNFR1β thus appears to have unstable transcription, perhaps resulting in decreased mRNA level. The aberrant polypeptides, if stable, could compete with full length gene products in receptor complex formation.

The 3.4 kb GmNFR1α promoter was delineated at its 5′ border by another ORF, representing a kinase domain of another LysM receptor gene. This was confirmed by full BAC sequencing. Microsynteny to a Medicago truncatula BAC clone furthermore showed that GmNFR1α was located in an equivalent position to MtLYK3, suggesting functional similarities.

The exon-intron structures of GmNFR1α and β are similar and showed high sequence identity (92% at nucleotide and 89% at amino acid level). Intron 6 of GmNFR1β is 374 bp shorter than that of GmNFR1α (FIG. 3A). Both soybean NFR1 genes are closely related to the LjNFR1, and MtLYK3 genes (FIG. 8 & FIG. 12) with amino acid identity of 79% and 75%, respectively. As expected, homology in the kinase domain was the highest, but notable sequence divergence was observed in the extracellular part containing possible Nod factor ligand binding sites, and thus controlling host range.

Genomic PCR products (at least 10 independent amplifications per genotype) of nod49, rj1, Clark (the wild-type near-isogenic parent of rj1), nod139, wild-type PI437.654 and Bragg were sequenced to determine accurately the site of mutation causing non-nodulation. Mutant nod49 is mutated in exon 5 of GmNFR1α through a T deletion (T986Δ of the coding sequence) leading to a shift in reading frame and subsequent protein termination within 5 amino acids (Acc. No.: DQ219807). The resultant protein, if stable, would lack the entire protein kinase domain and presumably any biological activity. Though unusual, the mutagen EMS was previously shown to induce single base pair deletions and subsequent ORF termination in the Arabidopsis pad3-1 mutation (Zhou et al., 1999, Plant Cell 11, 2419). Mutant rj1 is mutated in exon 4 of GmNFR1α by an A deletion (A769Δ) leading to protein termination within 51 amino acids (DQ219808). As for nod49, most of the kinase domain would be absent (FIG. 3B). Wild-types Bragg and Clark as well as mutants nod49, nod139 and rj1 contain identical wild-type GmNFR1β. Conversely, EMS mutant nod139 that lacks all symbiotic responses with B. japonicum (Mathews et al., 1990, supra) and was mapped to another location in the soybean genome has a wild-type GmNFR1α sequence. Reference wild-type cultivar PI437.654, used for BAC library construction (Tomkins et al., 1999, supra), also had wild-type GmNFR1α sequence (DQ219805).

GmNFR1β of Bragg, Clark, nod49 and rj1 are identical but differ from that of BAC54B21 through a SNP in exon 10 that leads to a nonsense mutation (Q513*; DQ219810) in PI437.654. Thus critical C-terminal portions are abolished, leading to complete loss of function similar to that seen in the nts382 (Q920*) mutation of the soybean NARK gene (6). The Q513* GmNFR1β mutation was confirmed in genomic DNA of PI437.654. Symbiosis competence tests show that PI437.654 is Nod⁺Myc⁺Fix⁺ indicating that the GmNFR1β mutation is completely complemented by a functional GmNFR1α.

To confirm that the sequenced alleles in GmNFR1α were causative for the non-nodulation phenotype of mutants nod49 and rj1, genetic complementation via high frequency hairy root transformation, followed by nodulation assays was conducted with Agrobacterium rhizogenes cucumopine strain K599 carrying the GmNFR1α cDNA driven by either its own 3.4 kb native promoter or the constitutive Cauliflower Mosaic Virus (CaMV) 35S promoter in binary vector pCAMBIA1305.1. Every plant (n>80) that formed roots (4-7 per plant) after transformation with K599 carrying the GmNFR1α gene developed nodules that were Nod⁺Fix⁺; as indicated by their red color, the healthy appearance of the plants (FIG. 4A), and total nitrogen gain compared to mutant or empty vector controls (Tables 1 & 2). In contrast, control roots formed with the empty vector failed to nodulate and resulted in yellow, nitrogen-deprived plants. Nodulation was variable, as about 40% of the roots formed on nod49 and rj1 plants failed to nodulate, presumably because of the lack of co-transformation of the Ri-plasmid and binary vector derived T-DNAs or gene silencing. Such roots were not considered in further quantitative characterization.

Transformed roots overexpressing the GmNFR1α gene from the 35S promoter possessed significantly higher nodule number, whether expressed per plant (Table 1A) or per unit root mass (Table 1B). Nodules were often clustered heavily in the upper root regions, suggesting that the success rate of nodulation is controlled by the strong expression of GmNFR1α. This phenotype did not occur when GmNFR1β was overexpressed. Overexpression of both GmNFR1α (40-45 fold) and β (70-80 fold) was confirmed by qRT-PCR (FIG. 16). The nodule-developing portion of the root (the nodulation interval) also increased slightly (54% compared to 45%) when composite nod49 and rj1 plants expressed 35SGmNFR1α. Overexpression of GmNFR1α in composite plants of wild type Clark or Bragg showed no statistically significant increase in nodulation per root, though a positive overall trend was seen.

As expected, soybean plants lacking the ability to nodulate and fix nitrogen (i.e., nod49) had a low nitrogen content (both in percentage and total terms) in contrast to the isogenic Bragg wild type (Table 2). When nod49 roots were transformed, vectors carrying the wild-type GmNFR1α gene complemented the nodulation and nitrogen fixation phenotype and led to increased nitrogen content. Complementation facilitated by the constitutive CaMV 35S promoter resulted in significantly higher plant nitrogen content compared to non-transformed wild type plants.

Reflecting an improved ability to interact with the Rhizobium-derived nodulation signal, soybean plants expressing the constitutive GmNFR1α gene construct formed increased numbers of nodules when inoculated with ultra-low Bradyrhizobium japonicum inoculation (10² cells per mL). Such conditions arise in soils suffering from abiotic stress (as seen in salt, moisture, or pH-stress conditions) or lacking prior history of compatible Bradyrhizobium cultivation (Table 3).

The here-described findings represent the first cloning, allele determination and functional complementation of a critical component for soybean NF reception. Ancestral genome duplication in soybean resulted in divergence of function for the two receptor kinases, although not to such a high extent as for the GmNARK/GmCLV1A genes (Searle et al., 2003, supra; Carroll et al., 1985, supra; Wopereis et al., 2000, supra; Krusell et al., 2002, supra; Nishimura et al., 2002, supra; Sagan et al., 1996, supra; Schnabel et al., 2005, supra). As shown by the nod49 and rj1 mutants, GmNFR1β alone does not facilitate the recognition of NF in epidermal and root hair cells to induce root hair deformation, curling and infection thread formation. In contrast, GmNFR1α alone (perhaps as seen in Lotus) does allow full symbiosis as shown by functional nodulation in the GmNFR1β Q513* mutant of PI437.654. However, GmNFR1β by itself (shown in the here-characterized GmNFR1α mutants) only sufficed to induce subepidermal CCDs in response to NF perception. Protein levels of GmNFR1β may be insufficient, based on reduced mRNA levels seen in qRT-PCR and caused by alternative splicing, leading to non-functional variants. Even 80 fold over-expression does not rectify this deficiency, suggesting that other evolutionary events forged the GmNFR1β protein to be a low efficiency receptor component. Irrespective of mechanism, we propose that GmNFR1α represents a higher efficiency NF receptor component than GmNFR1β.

If inoculated with high rhizobial titers (resulting in high localized NF concentration), GmNFR1α deficiency was partially suppressed as the GmNFR1β receptor component allowed normal infection and cell division stages, though sparingly. We tested this phenomenon by inoculating nod49 plants with different titers of B. japonicum CB1809 and observed increased partial nodulation success per plant with elevated rhizobial concentration. Addition of NF (NodV:MeFuc; 10 nM) to the nutrient medium significantly increased nodulation on nod49 mutant plants (Table 4). Since infection thread formation is essential for the progression of early CCDs (FIG. 4B), mutations in GmNFR1α result in non-nodulation. Thus GmNFR1α mutants, when exposed to high NF levels, form nodules via normal infection, showing that GmNFR1β suffices for all early nodule ontogeny steps.

The discovery of a critical NF receptor component of soybean opens new possibilities for optimizing this agriculturally important symbiosis. Many environmental conditions, such as water deficiency, nitrate, or soil acidity, and low bacterial inoculant number decrease nodulation and thus symbiotic nitrogen gain (Lawson et al., 1988, Plant & Soil 110, 123; Duzan et al., 2004, J. Exp. Bot. 55, 2641). Efforts to increase the amount of symbiotic plant tissue through alteration of autoregulation of nodulation have not yielded consistent agronomic advantages (Penmetsa et al., 2003, Plant Physiol. 131, 1), as supernodulated plants are commonly characterized by reduced root systems, especially when inoculated (Song et al., 1995, Soil & Environ. Biochem. 27, 563). Likewise improvements of commercial bacterial inoculants have been difficult to maintain in agronomic conditions because of competition from soil-adapted rhizobia. Since environmental stress effects on nodulation can be alleviated by increased NF levels (a seemingly unpractical agricultural procedure; see Lawson and Duzan referenced above), increased sensitivity to ‘natural’ NF concentrations, as described here, may lead to decreased stress effects on soybean nodulation and nitrogen gain. Discovery of a rate-limiting determinant of NF reception in soybean may also facilitate the construction of “exclusive symbioses”, comprising specifically designed bacterial-host combinations, and the manipulation of the host range for symbiotic nodulation.

### Example 2

### GmNFR5α and GmNFR5β

Isolation of the NFR5 Genes of Soybean.

The non-nodulating soybean mutants nod139 (Carroll et al, 1986, supra) and NN5 (Pracht et al., 1993) were not able to show the earliest morphological changes in response to rhizobial inoculation, such as the deformation and curling of root hairs, the initiation of subepidermal cell divisions and the formation of infection threads (Matthews et al., 1987, supra; Francisco et al., 1994). However, they established symbiotic interaction with mycorrhizal fungi indicating that the mutations affected an early, nodulation specific step of the symbiotic development (data not shown). Since mutations in the NFR1 and NFR5 genes coding for potential Nod Factor Receptors resulted in similar phenotypes (Ben Amor et al., 2003; Duc et al., 1989; Madsen et al., 2003, supra; Radutoiu et al., 2003, supra) we initiated a candidate gene approach instead of the more tedious map-based cloning. We designed an NFR5 specific primer pair (NFR5U/NFR5R in Table 6) to isolate and study the NFR5 gene of soybean. The amplified fragment of the soybean genome possesed high sequence similarity (84%) to the LjNFR5 gene and was used to screen filters containing a BAC library of soybean variety PI437.654 (Tomkins et al., 1999, supra). The HindIII fingerprinting of the isolated BAC clones, that had been confirmed by PCR to carry the NFR5 specific fragment, revealed two genomic environments and thus two copies of the gene in agreement with the duplicated nature of the soybean genome (Shoemaker et al., 1996). The nucleotide sequence of the two gene copies designated as GmNFR5α and GmNFR5β was determined by primer walking using the isolated BAC clones as template and proved to be 95% identical to each other.

FIG. 17 describes the GmNFR5α nucleotide sequence.

FIG. 18 describes the GmNFR5β nucleotide sequence.

FIG. 19 provides amino acid sequences of GmNFR5α protein and GmNFR5β protein while FIG. 20 provides an amino acid sequence alignment of GmNFR5α, GmNFR5β, LjNFR1 and MtLYK3 proteins.

Similarly to the orthologous sequences of other legumes, the GmNFR5 genes did not contain any intron and coded for receptor-like protein kinases possessing three extracellular LysM domains and lacking the conserved subdomain VIII of kinases. The NFR5 proteins of soybean shared 72-74% overall amino acid sequence identity with the Lotus, Pisum and Medicago sequences (FIG. 20). The sequence identity was higher (79-82%) in the transmembrane/kinase domains and lower (64-67%) in the extracellular domain which was believed to be responsible for the ligand binding and thus the determination of the host-range.

Widespread Distribution of a Retroelement Insertion in the NFR5β Gene of US Soybean Cultivars.

Genetic analysis of the mutants (Gresshoff and Landau-Ellis, 1994; Pracht et al., 1993) indicated that recessive alleles of two genes were responsible for the non-nodulation phenotype and one of the genes was non-functional in the parental lines.

Sequencing the alleles from the parental lines Bragg and Williams revealed that both of them carried a 1407 basepair long insertion sequence at the same position in the NFR5β gene. The insertion had the characteristics of a non-autonomous retroelement: it has long terminal repeats of 214 basepairs, a non-perfect duplication of the 11 basepair target-site and no footprint of protein coding sequence. Homology searches against public databases (GeneBank: non-redundant, htgs, gss, EST) revealed only limited similarity (80% identity over 300 nucleotides) to a genomic survey sequence of soybean. According to Allen and Bhardwaj (1987) cultivars Bragg and Williams were distantly related with two common ancestors, ancestral lines CNS and Illini which was an ancestor of S100.

To test the origin and distribution of the mutant allele a primer pair was designed to detect the insertion element in the NFR5β gene and an amplification experiment was performed using genomic DNA of ancestral, first and second generation soybean lines from the USA as well as DNA from cultivars from other countries as template. As expected, the fragment could be amplified from the parental lines and their mutants but was absent in the genome of G. soya and cultivar Harosoy63 which were shown to carry the wild type alleles of the two genes in the genetic experiments (Gresshoff and Landau-Ellis, 1994; Pracht et al., 1993). As for the ancestors of Bragg, we have genetic material from its parent Jackson and the sibling (Lee) of its other parent (D49-2491), as well as from S100 which was crossed with CNS to obtain Lee and D49-2491.

An amplification product of the same size as in Bragg, Williams and their mutants could be detected only in the case of Lee indicating that the origin of the mutant allele was line CNS. To our surprise, cultivar Wayne, the parent of Williams with CNS as an ancestor, did not carry the mutant allele, however, other ancestors like Clark and Richland, which have no known relation to CNS, posses the insertion sequence in the NFR5β gene. Analysis of the amplification results and the pedigree of the tested soybean cultivars revealed that at least five ancestral lines (CNS, Richland, Peking, Perry, and a parent or parents of Dorman: Dunfield and/or Arksoy) thought to be unrelated carry the same mutation. CNS, Richland, Peking and Dunfield are known to be of Chinese origin and thus might have common ancestors. Since these plants represent at least 20% of the genetic base of North American soybean lines (Gizlice et al., 1994) this result also means that the genetic diversity of these cultivars is even lower than predicted from the breeding data. Interestingly, although most of the non-US cultivars tested were devoid of the mutant allele, the Japanese cultivar Enrei of unknown pedigree also carried the mutation indicating common ancestors with the North-American lines.

Analysis and Complementation of the Mutants.

Sequencing the NFR5α gene from mutants nod139 and NN5 showed in both cases the presence of missense mutation in the coding sequence terminating the translation at the 338 and 502 amino acids, respectively, indicating that the lack of functional NFR5 proteins caused the mutant phenotype. To prove that the mutations in the NFR5 genes led to the nodulation failure we cloned the NFR5α and NFR5β genes of both G. max PI437.654 and G. soya into the binary vector pCAMBIA1305.1 and introduced them into the mutant plants via Agrobacterium rhizogenes mediated transformation. While transformation with the empty vector resulted in Nod⁻ phenotype (16 out of 20 plants carried transgenic roots based on GUS staining), the majority of the plants transformed with the gene constructs formed nodules on the hairy roots indicating successful complementation.

Throughout this specification, the aim has been to describe the preferred embodiments of the invention without limiting the invention to any one embodiment or specific collection of features. Various changes and modifications may be made to the embodiments described and illustrated herein without departing from the broad spirit and scope of the invention.

All patent and scientific literature, computer programs and algorithms referred to in this specification are incorporated herein by reference in their respective entireties.

TABLE 1
The effect of GmNFR1α gene expression on soybean nodulation.
(A) Average nodule number of composite soybean plants transformed
with the GmNFR1α gene. (B) Nodule number per root dry weight (mg⁻¹).
At least 20 replicates for each treatment were scored 35 days after
inoculation with Bradyrhizobium japonicum strain CB1809.

| | | GmNFR1α + | |
|---|---|---|---|
| | Empty vector | Native 3.4 kb | GmNFR1α + |
| | (no GmNFR1α) * | promoter | 35S promoter |
| A | | | |
| nod49 | 0.0 ^{a} | 139.4 ± 30.2 ^{c} | 278.5 ± 46.1 ^{d} |
| rj1 | 0.0 ^{a} | 87.8 ± 28.6 ^{b} | 211.7 ± 31.9 ^{c} |
| Bragg | 97.4 ± 25.1 ^{b} | 152.9 ± 36.6 ^{c} | 166.3 ± 29.5 ^{c} |
| Clark | 116.2 ± 8.8 ^{b} | 155.1 ± 20.1 ^{c} | 236.0 ± 37.7 ^{d} |
| B | | | |
| nod49 | 0 ^{a} | 5.0 ± 0.9 ^{c} | 10.3 ± 2.0 ^{d} |
| Bragg | 1.5 ± 0.2 ^{b} | 2.8 ± 0.3 ^{b} | 3.3 ± 0.3 ^{c} |

* numbers followed by the same letter for the same measured parameter are not significantly different at P = 0.05.

TABLE 2
Nitrogen status of the composite plants 48 days after
inoculation. (A) Relative (% of root dry weight) and
(B) total (mg/plant) nitrogen content of plants.

| | | GmNFR1α + | |
|---|---|---|---|
| | Empty vector | Native 3.4 kb | GmNFR1α + |
| | (no GmNFR1α) * | promoter | 35S promoter |
| A | | | |
| nod49 | 1.1 ± 0.0 ^{a} * | 2.5 ± 0.1 ^{c} | 2.8 ± 0.2 ^{d} |
| Bragg | 2.1 ± 0.1 ^{c} | 1.7 ± 0.1 ^{b} | 2.1 ± 0.1 ^{c} |
| B | | | |
| nod49 | 4.2 ± 0.4 ^{a} | 122.5 ± 7.9 ^{d} | 126.5 ± 8.2 ^{d} |
| Bragg | 54.5 ± 5.6 ^{b} | 85.0 ± 3.3 ^{c} | 74.2 ± 7.2 ^{c} |

* numbers followed by the same letter for the same measured parameter are not significantly different at P = 0.05.

TABLE 3
Overexpression of GmNFR1α permits nodule formation in
non-nodulation mutants nod49 and rj1 at low initial
Bradyrhizobium japonicum inoculum titers. Plants were
inoculated with B. japonicum CB1809 of different titers; values
are nodule number per plant. Plants were scored after 35 days, n = 8.

| | | | |
|---|---|---|---|
| B. japonicum | Empty vector | GmNFR1α + Native | GmNFR1α + |
| cfu · ml⁻¹ | (no GmNFR1α) * | 3.4 kb promoter | 35S promoter |

nod49

| | | | |
|---|---|---|---|
| 10² | 0.0 ^{a} * | 6.3 ± 3.6 ^{b} | 134.0 ± 25.4 ^{d} |
| 10⁵ | 0.0 ^{a} | 46.5 ± 9.5 ^{c} | 570.0 ± 40.1 ^{f} |
| 10⁷ | 0.0 ^{a} | 97.7 ± 25.4 ^{d} | 565.3 ± 54.6 ^{f} |

rj1

| | | | |
|---|---|---|---|
| 10² | 0.0 ^{a} * | 2.0 ± 1.2 ^{b} | 41.0± 15.3 ^{c} |
| 10⁵ | 0.0 ^{a} | 151.3 ± 34.2 ^{d} | 316.5 ± 28.5 ^{e} |
| 10⁷ | 0.0 ^{a} | 143.0 ± 27.0 ^{d} | 296.0 ± 35.8 ^{e} |

TABLE 4
Interaction of the initial Bradyrhizobium japonicum inoculum titer and the presence
of Nod Factor on nodule number. Plants were inoculated with B. japonicum strain
CB1809 of different titers, irrigated with NF (NodV-MeFuc) at 0 (No NF) or 10⁻⁸ M
(Plus NF) concentration. Plants were scored after 35 days. n = 10.

| B. japonicum | nod49 | | nod139 | | Bragg | |
|---|---|---|---|---|---|---|
| cfu · ml⁻¹ | No NF | Plus NF | No NF | Plus NF | No NF | Plus NF |
| 10³ | 0.0 ^{a} | 0.0 ^{a} | 0.0 ^{a} | 0.0 ^{a} | 79.2 ± 9.3 ^{d} | 82.5 ± 5.0 ^{d} |
| 10⁷ | 0.3 ± 0.2 ^{a} | 0.7 ± 0.2 ^{b} | 0.0 ^{a} | 0.0 ^{a} | 102.4 ± 8.8 ^{e} | 101.4 ± 8.5 ^{e} |
| 10¹⁰ | 0.8 ± 0.3 ^{b} | 2.3 ± 0.4 ^{c} | 0.0 ^{a} | 0.2 ± 0.2 ^{a} | 112.8 ± 9.5 ^{e} | 104.2 ± 6.9 ^{e} |

TABLE 5
Nucleotide sequence of GmNFR1 primers
(SEQ ID NOS: 20 to 54)

| | |
|---|---|
| Primer | Sequence |

Primer pairs to amplify probes and test BAC clones

| | |
|---|---|
| GmNFR1 Forw1 | GCTCTCCTTTTCGCATCATC |
| GmNFR1 Rev1 | CCAAGTTGAGCAATCTGCAA |
| GmNFR1 Forw2 | ATGCTTGGGGTTGTTTGAAG |
| GmNFR1 Rev2 | CAACGTGCTTCCAAAAGTCA |
| GmNFR1 Forw3 | CAGAAACTTGCCAATCCACC |
| GmNFR1 Rev3 | CCAAGTTGAGCAATCTGCAA |
| GmNFR1 Forw4 | GCCTTGATGCACAGTTGCTA |
| GmNFR1 Rev4 | CGTGCAAGCATCAACAGAAT |

Primer pairs for RT-PCR

| | |
|---|---|
| RT GmNFR1α-Forw | ATTCACGAGCACACTGTGCCT |
| RT GmNFR1α-Rev | GCCAAAATCTGCAACCTTTCC |
| RT GmNFR1β-Forw | ATTCACGAGCACACTGTGCCA |
| RT GmNFR1β-Rev | ACCAAAATCTGCAACCTTTCC |
| RT GmActin 2/7-Forw | GGTCGCACAACTGGTATTGTATTG |
| RT GmActin 2/7-Rev | CTCAGCAGAGGTGGTGAACA |

Primer to sequence BAC clones

| | |
|---|---|
| 55N1seq1 | AACACATGCCCCAGAAACTC |
| 55N1seq2 | TCAGGCCTGGGAATAATTTG |
| 55N1seq3 | TTGAACCCTCAATACGCTGA |
| 55N1seq4 | CTTTCAGAAAAACAGGTTTGG |
| 55N1seq5 | TCCGGGTAAAGTCTCTGGAA |
| 55N1seq6 | TGTGCAAGCATCGACAGAAT |
| 55N1seq7 | TTGGCATAAGCAGTTCGATG |
| 55N1seq8 | ATTCAGCAAGAGGCCTTGAA |
| 55N1seq9 | TGAACGGATCATAACGACGA |
| 55N1seq10 | CCAAGTTGAGCAATCTGCAA |
| 55N1seq11 | GCTCAACTTGGGAGAGCTTG |
| 54B21seq1 | GAGTTTCTGGGGCATGTGTT |
| 54B21seq2 | TCAGGCCTGGGAATAATTTG |
| 54B21seq3 | ACATGATGTGAAAAGGAGAGCA |
| 54B21seq4 | CTTGCAGAAAAACAGGTTTGG |
| 54B21seq5 | TCCGGGTAAAGTCTCTGGAA |
| 54B21seq6 | CGTGCAAGCATCAACAGAAT |
| 54B21seq7 | ATTCAGCAAGAGGCCTTGAA |
| 54B21seq8 | TTGATTGTGGAAAACGAGCA |
| 54B21seq9 | CCAAGTTGAGCAATCTGCAA |
| 54B21seq10 | GCTCAACTTGGGAGAGCTTG |

TABLE 6
Nucleotide sequence of GmNFR5 primers
(SEQ ID NOS: 55 to 76)

| | |
|---|---|
| NFR5U | ATTGCAAGAGCCAGTAACATAG |
| NFR5R | GTATGTTCATGCATGTATTGC |
| Nf5seq5pr | GATGTTGGCCAGCAAGCCG |
| Nf5seq3UTR | AAGTTGCAATTGACCTCAGAC |
| Nf5RTd | TAGGTTTCACATGAAGGCGGTG |
| Nf5PrD | GGGGATCCACCATTGCTGTTTAGTTGTGAACA |
| Nf5BinvHind | GGAAGCTTGGTTTAGGGGAGTGTG |
| Nf5Binv1 | GTCACTTCCATAGCAGCTCGTTGA |
| Nf5BinvUP | GTAAGGGAGGCCCTTGAGTCTG |
| Nf5inv2down | ACCTGTGGTTGCACTGGAAACC |
| Nf5seq5pr2 | GTATGCAATTCATGCGCATG |
| NF5AsacFW1 | GGGGAGCTCATATCAACAACTGCAGTTGCC |
| NF5AhindR | GGTATGAAACATAAGCTTAATGCAAT |
| NF5BsacFW1 | GGGGAGCTCATATCAACAACGGCAATTGCT |
| NF5BhindR | CATAAGCTTGATGCAACCAGTGGT |
| NF5kpnFW | AAAGGTACCCAAAGAAAAGGGTGCAAG |
| NF5Bseq3 | CACTCAAATGCCGTCCTTATC |
| Nfr5D1 | TCTGCAGAAGGTGAATCATG |
| Nfr5R2 | TTCATGCATGTACTGCAAACCC |
| Nfr5R3 | GCCAAGGAGGCCAAGCTGAG |
| Nfr5D2 | GCATTTGGGGTGGTTCTGA |

What is claimed:

1. An isolated nodulation factor (NF) receptor protein comprising an amino acid sequence selected from the group consisting of SEQ ID NO:1, SEQ ID NO:2, and SEQ ID NO:4.

2. An isolated nodulation factor (NF) receptor complex comprising a nodulation factor (NF) receptor protein that comprises an amino acid sequence selected from the group consisting of SEQ ID NO:1, SEQ ID NO:2, and SEQ ID NO:4.

3. The isolated NF receptor complex of claim 2, further comprising an isolated nodulation factor (NF) receptor protein comprising the amino acid sequence of SEQ ID NO:3.

4. An isolated variant NF receptor protein, wherein the variant NF receptor protein comprises an amino acid sequence selected from the group consisting of:
(i) an amino acid sequence that is at least about 80% identical to SEQ ID NO:1;
(ii) an amino acid sequence that is at least about 90% identical to SEQ ID NO:2; and
(iii) an amino acid sequence that is at least about 90% identical to SEQ ID NO:4.

5. A protein fragment of the isolated NF receptor protein of claim 1, which protein fragment is encoded by one or more exons of a nodulation factor (NF) receptor gene.

6. An isolated nucleic acid that encodes an amino acid sequence of a nodulation factor (NF) receptor protein, variant thereof, or protein fragment thereof, wherein the NF receptor protein, variant thereof, or protein fragment thereof is selected from the group consisting of: (a) an amino acid sequence according to SEQ ID NO:1; (b) an amino acid sequence according to SEQ ID NO:2; (c) an amino acid sequence according to SEQ ID NO:4; (d) an amino acid sequence that is at least about 80% identical to SEQ ID NO:1; (e) an amino acid sequence that is at least about 90% identical to SEQ ID NO:2; (f) an amino acid sequence that is at least about 90% identical to SEQ ID NO:4; and (g) a protein fragment of any one of (a)-(f) that is encoded by one or more exons of a nodulation factor (NF) receptor gene.

7. The isolated nucleic acid of claim 6, wherein the isolated nucleic acid comprises a nucleotide sequence selected from the group consisting of SEQ ID NO:5, SEQ ID NO:6, SEQ ID NO:8, SEQ ID NO:9, SEQ ID NO:10, and SEQ ID NO:12.

8. A gene fragment of a gene encoding the isolated NF receptor protein of claim 1, or the isolated variant NF receptor protein of claim 4, wherein the gene fragment comprises an intron or exon of said gene.

9. A promoter-active fragment of a gene encoding the isolated NF receptor protein of claim 1 or the isolated variant NF receptor protein of claim 4.

10. A chimeric gene comprising the promoter-active fragment of claim 9, and a heterologous nucleic acid operably linked to said promoter-active fragment.

11. A genetic construct comprising the isolated nucleic acid of claim 6, wherein the isolated nucleic acid is operably linked to one or more regulatory sequences.

12. A genetic construct comprising the promoter-active fragment of claim 9.

13. The genetic construct of claim 12, wherein said promoter-active fragment is operably linked to a heterologous nucleic acid.

14. A genetically-modified plant, plant cell, or plant tissue comprising the genetic construct of claim 11.

15. A genetically-modified plant, plant cell, or plant tissue comprising the genetic construct of claim 12.

16. The genetically-modified plant, plant cell, or plant tissue of claim 14, wherein the genetically-modified plant, plant cell, or plant tissue displays one or more altered characteristics as compared to a plant that did not have the genetic construct introduced into it, which altered characteristics are selected from the group consisting of: (1) improved, enhanced, or otherwise facilitated nodulation or nitrogen fixation; (2) reduced nodulation or nitrogen fixation; and (3) enhanced acid tolerance.

17. A method of producing a genetically-modified plant, plant cell or plant tissue including the step of introducing the genetic construct of claim 11 into a plant cell or plant tissue.

18. A method of producing a genetically-modified plant, plant cell, or plant tissue including the step of introducing the genetic construct of claim 12 into a plant cell or plant tissue.

19. The method of claim 17, wherein the genetically-modified plant, plant cell or plant tissue displays one or more altered characteristics as compared to a plant that did not have the genetic construct introduced into it, which altered characteristics are selected from the group consisting of: (1) improved, enhanced, or otherwise facilitated nodulation or nitrogen fixation; (2) inhibited, diminished, or otherwise reduced nodulation or nitrogen fixation; and (3) enhanced acid tolerance.

20. The method of claim 18, wherein the genetically-modified plant, plant cell, or plant tissue displays one or more altered characteristics as compared to a plant that did not have the genetic construct introduced into it, which altered characteristics are selected from the group consisting of: (1) improved, enhanced, or otherwise facilitated nodulation or nitrogen fixation; (2) inhibited, diminished, or otherwise reduced nodulation or nitrogen fixation; and (3) enhanced acid tolerance.

21. An antibody or antibody fragment that binds the isolated nodulation factor (NF) receptor protein of claim 1.


SEQUENCE LISTING (86 sequences)

SEQ ID NO: 1 - protein, 611 aa, Glycine max
MELKKGLLVFFLLLECVCYNVESKCVKGCDVAFASYYVSPDLSLENIARLMESSIEVIISFNEDNISNGYPLSFYRLNIPFPCDCIGGEFLGHVFEYSASAGDTYDSIAKVTYANLTTVELLRRFNGYDQNGIPANARVNVTVNCSCGNSQVSKDYGMFITYPLRPGNNLHDIANEARLDAQLLQRYNPGVNFSKESGTVFIPGRDQHGDYVPLYPRKTGLARGAAVGISIAGICSLLLLVICLYGKYFQKKEGEKTKLPTENSMAFSTQDVSGSAEYETSGSSGTASATGLTGIMVAKSMEFSYQELAKATNNFSLENKIGQGGFGAVYYAELRGEKTAIKKMDVQASTEFLCELKVLTHVHHFNLVRLIGYCVEGSLFLVYEYIDNGNLGQYLHGTGKDPLPWSGRVQIALDSARGLEYIHEHTVPVYIHRDVKSANILIDKNIRGKVADFGLTKLIEVGGSTLHTRLVGTFGYMPPEYAQYGDISPKVDVYAFGVVLYELISAKNAVLKTGESVAESKGLVALFEEALNQSNPSESIRKLVDPRLGENYPIDSVLKIAQLGRACTRDNPLLRPSMRSIVVALMTLSSPTEDCDTSYENQTLINLLSVR

SEQ ID NO: 2 - protein, 618 aa, Glycine max
MELKKWLLFFLLLEYVCCNAESKCVKGCDVALASYYVSPGYLLFENITRLMESIVLSNSDVIIYNKDKIFNENVLAFSRLNIPFPCGCIDGEFLGHVFEYSASAGDTYDSIAKVTYANLTTVELLRRFNSYDQNGIPANATVNVTVNCSCGNSQVSKDYGLFITYPLRPGNNLHDIANEARLDAQLLQSYNPSVNFSKESGDIVFIPGRDQHGDYVPLYPRKTGLATSASVGIPIAGICVLLLVICIYVKYFQKKEGEKAKLATENSMAFSTQDVSGSAEYETSGSSGTASTSATGLTGIMVAKSMEFSYQELAKATNNFSLENKIGQGEFGIVYYAELRGEKTAIKKMDVQASTEFLCELKVLTHVHHLNLVRLIGYCVEGSLFLVYEYIDNGNLGQYLHGTGKDPFLWSSRVQIALDSARGLEYIHEHTVPVYIHRDVKSANILIDKNFRGKVADFGLTKLIEVGGSTLQTRLVGTFGYMPPEYVQYGDISPKVDVYSFGVVLYELISAKNAVLKTGESVAESKGLVALFEEALNQSNPSESIRKLVDPRLGENYPIDSVLKIAQLGRACTRDNPLLRPSMRSIVVALLTLSSPTEDCYDDTSYENQTLINLLSVR

SEQ ID NO: 3 - protein, 598 aa, Glycine max
MAVFFPFLPLHSQILCLVIMLFSTNIVAQSQQDNRTNFSCPSDSPPSCETYVTYIAQSPNFLSLTNISNIFDTSPLSIARASNLEPMDDKLVKDQVLLVPVTCGCTGNRSFANISYEINQGDSFYFVATTSYENLTNWRAVMDLNPVLSPNKLPIGIQVVFPLFCKCPSKNQLDKEIKYLITYVWKPGDNVSLVSDKFGASPEDIMSENNYGQNFTAANNLPVLIPVTRLPVLARSPSDGRKGGIRLPVIIGISLGCTLLVLVLAVLLVYVYCLKMKTLNRSASSAETADKLLSGVSGYVSKPTMYETDAIMEATMNLSEQCKIGESVYKANIEGKVLAVKRFKEDVTEELKILQKVNHGNLVKLMGVSSDNDGNCFVVYEYAENGSLDEWLFSKSCSDTSNSRASLTWCQRISMAVDVAMGLQYMHEHAYPRIVHRDITSSNILLDSNFKAKIANFSMARTFTNPMMPKIDVFAFGVVLIELLTGRKAVTTKENGEVVMLWKDIWKIFDQEENREERLKKWMDPKLESYYPIDYALSLASLAVNCTADKSLSRPTIAEIVLSLSLLTQPSPATLERSLTSSGLDVEATQIVTSIAAR

SEQ ID NO: 4 - protein, 599 aa, Glycine max
MAVFFSFLPLRSQILCLVLMLFFTNIVAQSQQTNETNFSCPSDSPPPSCETYVTYIAQSPNFLSLTSISNIFDTSPLSIARASNLEPEDDKLIADQVLLIPVTCGCTGNRSFANISYEINPGDSFYFVATTSYENLTNWRVVMDLNPSLSPNTLPIGIQVVFPLFCKCPSKNQLDKGIKYLITYVWQPSDNVSLVSEKFGASPEDILSENNYGQNFTAANNLPVLIPVTRLPVLAQSPSDVRKGGIRLPVIIGISLGCTLLVVVLAVLLVYVYCLKIKSLNRSASSAETADKLLSGVSGYVSKPTMYETDAIMEATMNLSEQCKIGESVYKANIEGKVLAVKRFKENVTEELKILQKVNHGNLVKLMGVSSDNDGNCFVVYEYAQNGSLDEWLFYKSCSDTSDSRASLTWCQRISIAVDVAMGLQYMHEHAYPRIVHRDIASSNILLDSNFKAKIANFSMARTFTNPTMPKIDVFAFGVVLIELLTGRKAMTTKENGEVVMLWKDIWKIFDQEENREERLKKWMDPKLESYYPIDYALSLASLAVNCTADKSLSRSTIAEIVLSLSLLTQPSPATLERSLTSSGLDVEATQIVTSIAAR

SEQ ID NO: 5 - DNA, 6383 bp, Glycine max
aataaaatattaattatgcttttcaactatatcaatatagtttatagtatctatattaggtgaaatgaagagcattaacgaatcaagagataatataattaattaaataagtatatattcttttaattgatcgtgtttatgaatttattctattttttaaacaattgtatccttcacaagtgccgtgaagcactctttagcatttctagtaaagccaacaataaattatacagagatgtgcgactatgcaatcggtgatatcacacagattcctttttgtttgttattagtgaagtcaatgaagtatattgggtcatagccaagctgcacaggcgtgcctcaaaatttaaaatgcaaaattgttctgtgtttgttagaacaatgagaagacgcgataagaagtggtttgttggcacattggccgacatggttggcatttcggatacaaaggattaaacaaagccagcattctcaatcacaaagattccccttgtcgttctgtatccctctctaccatattcaatgtacaccaaatatgcccttaataaataaaatggcatgcaagttgttacccaagcatgcaataaataaatgacatgcaagtcaactacaataattttctagcctatcctactgtttccttccacactctcattgaaactgtaaatggtataacctatcaggtgttagttctaaaacaggcataaacgtgtgcatatgaattcatatactctaacacaaatttcggacaccactaatatctaaaatataggtatttgggtactacttacactcacaaataaagagattctaatcaaatgaaaaattaataacatataataaatcaaatatctaaattgatgttatattcatctatttaaaccagttttaatttttatgtttttcaagtgtattaattgtgtaaaggtgacgccttaagtgtttaagtcaataaagagtaatttttgaaccagacacctaataagaagtgttaaacaagtgtccaggtgtatcggtgttgaaaacatatatgaaacaacgacacttcaaacaagcaaggcctccgtgtttcataggtttaatgttcgcacgcattcacttaagttacctacaacattcttttatgttttagtgattaaaagaggaagtgtgacattggtttcaactttgaagagaaaaagaaatgaaaataattattgattaaaccctgtatagaaagtcctagaaatcttgttttctgatttggatttctttgtgttcctcttatttgctccctgtgatccaatggaactcaaaaaagggttacttgtgttctttttgctgctggagtgtgtttgttacaatgtggaatccaagtgtgtgaagggatgtgatgtagctttcgcttcctactatgtcagtccggatttaagcttagaaaatatagcgcggttgatggaatcaagcattgaagttataatcagcttcaatgaagacaatatatcgaatggttatccgctatccttttacagactcaatattccattcccctgtgactgtattggtggtgagtttctggggcatgtgtttgagtactcagcttctgcaggtgacacctatgattcgattgcgaaagtgacatacgccaatctcaccaccgttgagcttttgcggaggttcaatggctatgatcaaaatggtatacctgcaaatgccagggttaatgtcacggtcaattgttcttgtgggaacagccaggtttcaaaagattatgggatgtttattacctatccactcaggcctgggaataatttgcatgatattgccaatgaggctcgtcttgatgcacagttgctgcagcgttacaatcctggtgtcaatttcagcaaagagagtgggactgttttcattccaggaagaggtatgctctccttttcgcatcatcaatgtattttttgatgtggacaaaacttagatacaactccttaggtgtttttgatgttgttctctaatcggatttggtgtttcacttcggtaagctattctaaatttctaatatttaatgcaaatctttataacatgaattatcaagatgaacctgcaattctgaatagagagcaatgcctctaagttattttccttttggtattatcagcatattgagggttcaaataactcatttatttttctgagtgtttgggataacattttatgtatttgtctaacgtttcaattttatttaacttgccagatcaacatggagactatgttcccttgtacccgaggtgggtaattttgattgtatcacctttcatgctgaattatgcacttacaattgaatatagctacatgtttgattctatctttttaactttcattttcttttccatctttcagaaaaacaggtttggatctcaaacttcatagagagttggttacatgaggattattttcagcttgatgttcacataaatatgagaaagaaagaaaaatcagagcctcatagattaaatttgcttctgtatataagcaggtcttgctaggggtgctgcagttggaatatctatagcaggaatatgcagtcttctattattagtaatttgcttatatggcaagtacttccagaagaaggaaggagagaaaactaaattgccaacagaaaattctatggcgttttcaactcaagatggtacgggtaaattttcgtattcatataacgcattcctttcaaactattcacattacatattcccagatatgggtgaaagttagtactctgaattttcatgtctttaagcttttgttatactatcttttttttttctttcaaagattatcctgtataaacttattacctgattaaattttaggctgttttaccttgtttcagaggtagaaaaattaacccttattttcttttacacattctcctcttagtcttgtatcccttgtaaaaaaaaaaaggccagctatttatcaacctcttcaaagtttacatgtcatcaactctggcatattcctagaattttatgtgtactagactactagcttaatggccatggtaacactttttgatttttcttactcttccgggtaaagtctctggaagtgcagaatatgaaacttcaggatccagtgggactgctagtgctactggcctcacaggcattatggtggcaaaatcaatggagttctcatatcaagaactagccaaggctacaaataactttagcttggagaataaaattggtcaaggtggatttggagctgtctattatgcagaactgagaggcgaggtatgaagttacatctatattcagttctataacataagcagacaaaaaacatattaatggaaagaaggaaaaacaaaaatggagatgatagaagtttttacttttactcgggtgatgttattagtgaaacatacggctttcccatgttattgctattttacatcagaaaagtagggatatgcttaaattgtaaggctttcagttcattgtgtggtatataagtttttacagtagactattcattgaactgaaaggtacatatggcattgttcacttattagtggataatattctcaagggtggattgacaagtttctggcttttatgccgtttcaggttaaccgtttaccttttctactctattcctggatttcctctcatataattcatttctatgcagaaaactgcaattaagaagatggatgtgcaagcatcgacagaatttctttgcgagttgaaggtcttaactcacgttcatcactttaatctggtacagcatccttcaaacaaccccaagcatgtatatatctgggaaggataattaatcattttctgtatagtttgaaaaacaataaggcagttagaaaaaaaaaaatatccagggtgattttgtgaacagaattgcaaaacagtctataattatccagcaaaattatttctgcagatccacgtgaaaatcctacaaattaacaagagatcagcattgcttgtgtaaaaaaacatgcaatatctttatcttacttctgtatttgtttgtgagcatcaatgtagtttatttttttggcataagcagttcgatgtaagttcaatatcattgttgtaaaggagaagattataggaagtatttgagaaaaatgaaggaggagagaatatttgaaagaaggctagtttttatgacttgagaaaagcttttgttttgacttcttttggtttcattgatttctttaaaaacgacaacctctcccccttttatagacttcaagggagagttctagatacatcaaaaaagattctacacatttcaagggagagttctacatgcttctcccaactacttagttctacacattccttccaattaaatattacttctacttatttctacacttctctagaagtttctttagagtagtagcacaaacttaattggcctaacacttagactaaatcaagtttatattattcaacaatttctgtatttatataactaccttttgtgaacaacaacacaggtgcgcttgattggatattgtgttgagggatcccttttccttgtatatgaatatattgacaatggaaacttaggccaatatttgcatggtacaggtgagaacagcatacattaataatattttcctgtgatgtttcatgttaccttattgtcaaacaataaataatgatgatagcatgattccagggaaagatcctttgccatggtctggtcgagtgcaaattgcgctagattcagcaagaggccttgaatatattcacgagcacactgtgcctgtgtatatccatcgtgatgtaaaatcagcaaatatattaatagacaagaacatccgtggaaaggttgcattgttatcattcttcatgatcctcactccacatcctgatttttcatatttttttagactaaaccgtgtaatcttttaattacaggttgcagattttggcttgaccaaacttattgaagttggaggctccacacttcacactcgtcttgtgggaacatttggatacatgccaccagagtatgatttgtttattgtgctaaataatcaaaacgaaatttcggttttgttgggaaaaaaaacatgtgttctctgtgttgttaatagtaggccctcttattattgatgaatcattagttgatgttattgatgaacggatcataacgacgaggaaaatattgtatgattaactagtaaaatcaaattcagttttagtaacatatcattgttacttagttcattaattatctcttttaatttttgcaaggatattactaggtttgtttttccatggattagagatcttatcttaatctttttaattgtggaaaacgagccctttagttttaattttgtatgaacaaaacttattttattgattacctggatttcctgcagatatgctcaatatggtgacatttctccaaaagtagatgtatatgcttttggagtggttctttatgaacttatttctgcaaagaatgctgttctaaagacaggtgaatctgttgctgaatcaaagggccttgtagctttggtgagtttgcatactccttctatgaaccagtttactaacaaaacactctcaattcacagaaaggaagttaaggttgacttgttttgtatttcagtttgaagaagcacttaatcagagtaatccttcagaaagtattcgcaaactggtggatcctaggcttggcgaaaactatccaattgattcagttctcaaggtggaagcatttttctgtgaaaataatttgaatatttatatcttatacaactttatcccagccaaatcttaaggtaagttgattgtttgatgagttgcagattgctcaacttgggagagcttgtacaagagataacccactactacgcccgagtatgaggtctatagttgttgctctcatgacactttcatcacctactgaggattgcgacacttcctacgaaaatcagactctcataaatctactgtctgtgagatgaaggttctttataacgattacaccatgtttttaatgacttttggaagcacgttgtgcattgtttgacaagtttgtacatgcatgaatggagttgagatttttgtaaatgagttttctacaattttcttctatctgatttgaaaactcctgttttgactcctaatagaaggtttttttttaaggctcaacttttttagatctcaatttttaatcattcaaaagttttttttaacaaattttagttcttggttaattttctgagtattttttagttcctcaacttttttttgtttatttttagtctctcatttatctttaatgcttaagataagaatttgttttgtccatctttaatctctcatttttatatattttgaactaattttgaa

SEQ ID NO: 6 - DNA, 5630 bp, Glycine max
aaaactggttcataagggggtggtctacccaactatataagcacttatcatattcatgaattactcgatgtgagactattcttaacatttgttatgtcaacggagtatatttggtcatagccaagctgcacaggcgtgcctcaaaatttaaaatggaaaattgttcttcgtttgttatgttagaacaatgagaggacacaatacgaagtggtttgttggcacattggccgacacggttggcatttcggatagaaaggatcaaacaaagccaacattttcaatcacagagatttccgcgtccatattatgcagccctctctaccataaaaaatatcactatattcaaagtacaccaaatatgtcctcctcaataaatgacatgcaagttgttatccaaaattaaataaataaataaattagggttcttgctaatagggtattggttaaggaattaaaacgagaaaatatttaatgtaaaaaccataagagaacataaaaaagtcaagtaaaacataattttgtgcatttgaataaatttttttttcttttagtttcttaatcaatatcttaagaacaccgatcaatatttgtcatataaataaatgacatgcaagtcaacttcaataattttctagccaatcctactgtttccttccacattctcgtggaaaactatttagcgttataacctatcaggtgtatgttctgaaaaaactaaaaagcataaacgtgtgcatgtgaattcttagtttatgttcattcacttaattagttacacctttatacttttattttatgttttgagttacttttctatagtctgtgtgttaattaaaagaggaagtgtgacattggtttcaagagaaaaaagaaatgaatatgattaaggctggtgtataaagtcctagaaatccacttttctgatttgagtttctttatgtctctcttgtgtgctctccgtgacccaatggaactcaaaaaatggttactgttctttttgttgctggagtatgtttgttgcaatgcggagtctaagtgtgtgaagggatgtgatgtagctttagcttcatactatgttagtccagggtatttactcttcgaaaatataacgcgcttgatggaatcaattgttctgtccaattctgatgttataatctacaacaaagacaaaatattcaatgaaaatgtgctagcattttccagactcaatattccattcccctgtggctgtatcgatggtgagtttctggggcatgtgtttgagtactcagcttctgcaggtgacacctatgattcgattgcgaaagtgacatatgccaatctcaccactgttgagcttttgcggaggttcaacagttatgatcaaaatggtatacctgcaaatgccacggttaatgtcacggtcaattgttcttgtgggaacagccaggtttcaaaagattatgggctgtttattacctatccactcaggcctgggaataatttgcatgatattgccaacgaggctcgccttgatgcacagttgctacagagttacaatcctagtgtcaatttcagcaaagagagtggggatattgttttcattccaggaagaggtatgctctccttttcacatcatgttattttggtgtactcatcaatgtatttttttggtatggacaaaacttagagtcttagatacaactccttaggtgtttttggtattgttctctaaaccaaattggtgtttcactttggtaagctattctaatatttaatgcaaacctttataacgtgaattatcaagatgaacctgcaattctgaatagagagcaatgtcaagttattttccttttggtattatcagcatattgagggttaaaataactcatttatttttcttcaaagcatttgggaaaacattttatgcatctgtctaacgtttcaattttatttaacttgccagatcaacatggagattatgttcccttgtaccctaggtgggtaattttgattgtctcacctttcatgctgaattatgctcttagaattgaatattgctacgtgcttgattctatctttttaactttcattttcttttccatcttgcagaaaaacaggtttggctctcaaacttcatagagagttggttacatgaagattattttcagcttcacaaaatatgagaaagcaaaaaaaaaaagaagtcagagcctgggagcttaaatttgcttctgtatataagcaggtcttgctacgagtgcttcagttggaatacctatagcaggaatatgcgttcttctattagtaatttgcatatatgtcaagtacttccagaagaaggaaggagagaaagctaaattggcaacagaaaattctatggcgttttcaactcaagatggtatgggtaaactttcgtattcatataacgcattccttctaaactattcacataacatattcccaaatatgggttaaagatagtactctgaattttcatgtctttaagcttttgttatactatcttttttttttctttcaaagattatcctgtataagtttattacctgatccaattttaggctgttttatcatttttcatgttttttctttaacacattctcctcttagtcttgtatctattataaaaaaaaaaatgcctgctatttatcaacctcttcaaagtttacttgtcatcaactctggcatattcctagaatttgatatgtactagactaatggggccatggcaacacttgttgatttttcttcctcttccgggtaaagtctctggaagtgcagaatatgaaacttcaggatccagtgggactgctagtactagtgctactggccttacaggcattatggtggcaaaatcaatggagttctcatatcaagaactagccaaggctacaaataacttcagcttggagaataaaattggtcaaggtgaatttggaattgtctattatgcagaactgagaggcgaggtaggaagttacatgtatattcagttctataacataatcagacaaaagaatattaatggaaagaaggaaaacaaaaatggaggatagaagtttttacttttactcgtgtgttgctattactgacacatacagttttcccatgctattgctattttacatcagaaaagtactgatatgtttaaattgtaaggctttcagttcattgtgtgatatataagttatataatttagttaatagtataagacaattcattgaactgaaaggtacctatggaattgttcacttattagttgataatattgtcaagggtggattggcaagtttctggcttttatgccatttcaggttaaccctttaccttttttactctattcctggatatgctctcatataattcatttctatgcagaaaactgcaatcaagaagatggacgtgcaagcatcaacagaatttctttgcgagttgaaggtcttaactcatgttcatcacttgaatctggtataacatccttcaaataactccaagcatgtattatgtatatatctgggaaggataattaatcattttccgtatagtttgaaaaacaataaggaagttaggaaaaaatatccagggtgattttgtgaacagaattgcaaaaacagtctataattatcctgaaatattatttctgcagatccacatgataatcctgcaaattaacatgagatcagcattacttgtgtgaaaaaaacttgtgatatctatatcttattcctgtaattgattgtgagcgtcaatgtagtttatttttttggcataagcagttccatgtaagttcaatacctttttctgtatttttatagctacctttttgtgaacaacaatacaggtgcgcttgattggatattgtgttgaggggtctcttttccttgtatatgaatatattgacaatggaaacttaggccaatatttgcatggtacaggtgagaacagcatgtatttatgatatttttcctatgatgtttcatgttaccttattgtcaaacaatgaataatgatgataacatgattccagggaaagatcctttcctatggtctagccgagtgcaaattgcactagattcagcaagaggccttgaatatattcacgagcacactgtgccagtgtatatccatcgtgatgtaaaatctgcaaatatattaatagacaaaaacttccgtggaaaggttgcattgttaccattcttcctgatcctttcttcaaatcattattttccatttctgttttgagactaaaccatgtctgcttttaaatacaggttgcagattttggtttgaccaaacttattgaagttggaggttccacacttcaaactcgtcttgtgggaacatttggatacatgccaccagagtatgatttgttctgttgtgttaaataatcaaaatgaaatttcggttttgttggaaaaaacatgtgttctctgtattgttaatagtaggccctcttattattgatgaatcgtaagttgatgttattgatgaacagatcacaacaacaagggaaatgttgtatgattaactagtaaaatcaaattcagttttagtgacatatcattgataattagttcattaattatctcttttaatttttgcaaggatattactaggtttgtttgtccatggattagcgatcttatcttaaactttttgattgcggaaaacgagcactttagttttaattttgtatgaacagaactaattattttattgattacctgaattttctgcagatatgttcaatatggtgacatttctccaaaagtagatgtatattcttttggagttgttctttatgaacttatttctgcaaagaatgctgtcctaaagacaggagaatctgttgctgaatcaaagggccttgtagctttggtgagtttacatactccttctctgaactgaactagtttactaacaaaataccctcaattcacagaaaggaagttacagttgacttgttttgtatttcagtttgaagaagcacttaatcagagtaatccttcagaaagtattcgcaaactggtggatcctaggcttggagaaaactatccaatcgattcagttctcaaggtggaagcatttttctgtgaaaataatttgattatttatatcttatacagttttataccaaccaaaacttaaggtaagttgattgtttgatgagttgcagattgctcaacttgggagagcttgtacaagagataacccactactacgcccgagtatgaggtctatagttgttgctctcttgacactttcatcacctactgaggattgctatgatgacacttcctacgaaaatcagactctcataaatctactgtctgtgagatgaaggttctttgtgacaattacaccatgtttttaatgagttttggaagcactttatgtaaggtctgaaaagtttgtacatgaatggagttgagatttttgtaaatgagttttgttcaattttcttctatctgatttggaaacacctgttgttctgactcctaatagaagtttttatttatcagcgaatattaatttgttggaatgttagttttctgagaagagaagatcgaactcaccatcttttctttcttcttttcttccttaaccatctggtccatcttatatt

SEQ ID NO: 7 - DNA, 3474 bp, Glycine max
tcaggtactcaaagaaaagggtgcgagaacgacattgagagagtaacataaggacggcattcaagggaaccatcaatctgatccttgagatatgattctctctcattaatagtccttaaagtaagaaaaactacttatatagttctaaaagttttagaaattataccaaataatttcttaaatattgaaaaaccctttaaattgatctttgaactttactaaaataaatcatcaatttaaggactaaactaatgggtattcaattcatatcaagtactaggctactacaaccataatcctattctttgatgtacgtcttgctcagctgctgaagacagtccagtattgagtttctttgattaaagataaaatgaaggtgaatttgatgaagtgctttagtatgttgaatcctatgcaaatggacaattcaacactccaagctgtgtaaattacaatagcaaataatggtctctgtccttgttaaaaattatcgagtttagttggtctgtaggtgtcggcttgctggccaacatcgtgcatagataaaacattaactggccgtccaaaggttggattaggcattgcaatactcttattgtcatttttttttatagcatgcgcatgaattgcatacaattaggctaatataatattgacgtgtccacagtgtcaaagattcggaaaaccaaaagaaaatatagttaaagctaatgacaaagacatgagcagatttttatatatattaaaccaaaggctatttttttgttgacaaagaatgcttcatacatatcaacaactgcagttgcctgtgataatagactctccttattctttccctcgttacttacatttgttcacaactaaacagcaatggctgtcttctttccctttcttcctctccactctcagattctttgtcttgtgatcatgttgttttccactaatattgtagctcaatcacaacaggacaatagaacaaacttttcatgcccttctgattcaccgccttcatgtgaaacctatgtaacatacattgctcagtctccaaattttttgagtctaaccaacatatccaatatatttgacacaagccctttatccattgcaagagccagtaacttagagcctatggatgacaagctagtcaaagaccaagtcttactcgtaccagtaacctgtggttgcactggaaaccgctcttttgccaatatctcctatgagatcaaccaaggtgatagcttctactttgttgcaaccacttcatacgagaatctcacgaattggcgtgcagtgatggatttaaaccccgttctaagtccaaataagttgccaataggaatccaagtagtatttcctttattctgcaagtgcccttcaaagaaccagttggacaaagagataaagtacctgattacatacgtgtggaagcccggtgacaatgtttcccttgtaagtgacaagtttggtgcatcaccagaggacataatgagtgaaaacaactatggtcagaactttactgctgcaaacaaccttccagttctgatcccagtgacacgcttgccagttcttgctcgatctccttcggacggaagaaaaggcggaattcgtcttccggttataattggtattagcttgggatgcacgctactggttctggttttagcagtgttactggtgtatgtatattgtctgaaaatgaagactttgaataggagtgcttcatcggctgaaactgcagataaactactttctggagtttcaggctatgtaagtaagcctaccatgtatgaaactgatgcgatcatggaagctacaatgaacctcagtgagcagtgcaagattggggaatcagtgtacaaggcaaacatagagggtaaggttttggcagtaaaaagattcaaggaagatgtcacggaagagctgaaaattctgcagaaggtgaatcatgggaatctggtgaaactaatgggtgtctcatcagacaatgatggaaactgttttgtggtttatgaatacgctgaaaatgggtctcttgatgagtggctattctccaagtcttgttcagacacatcaaactcaagggcatcccttacatggtgtcagaggataagcatggcagtggatgttgcgatgggtttgcagtacatgcatgaacatgcttatccaagaatagtccacagggacatcacaagcagtaatatccttcttgactcgaactttaaggccaagatagcaaatttctccatggccagaacttttaccaaccccatgatgccaaagatagatgtctttgcatttggggtggttctgattgagttgcttaccggaaggaaagccatgacaaccaaggaaaatggtgaggtggtcatgctgtggaaggacatttggaagatctttgatcaagaagagaatagagaggagaggctcaaaaaatggatggatcctaagttagagagttattatcctatagattacgctctcagcttggcctccttggcggtgaattgtactgcagataagtctttgtccagaccaaccattgcagaaattgtccttagcctctcccttctcactcaaccatctcccgcaacattggagagatccttgacttcttctggattggatgtagaagctactcaaattgtcacttccatagcagctcgttgattgagtgaaggaaatttagtttctcaaatccatgatggtattttgtttacatgatgattattacatctttagtcattaatggttggcttggtttgggggagtgtgttcaaaatttcgtttttttccatccctgttattttttttaagtttggggtagagtcagcaaaaatggaagttgcaattgacctcagactaaacttgcttatttccctgtatcttttttgtgtgataattgaaactgaattatatgatggattatctgttacatgtacaaacaaattcaagcgagaaaaaatgattgagtttgaaatatacgtttctgccactgattgcattaagcttatgtttcatacctcataaagtcacaatactgcacggatagaatttaggattttgttcatccaattacatcctcaattcttcttatctaggtactttttgccattaacacttggatcgctacaatacaattaatctatcccacttttttgtcttctaattttttgtcacaaggctggacattgaaacttaatggagaatttatgcaagaaggcctttggatgcggcctcagctctgttaaattattattattgtatgtctttaaaattgagagtgtatggcctataatatctgctcatattcttgactacaatgccaatcccttggtgaaccttcatccatatctcacagcccgcattaaggaatagatgcatttttaacgtatattgatgcatggagtaaccaaaggtaaaaagtgcaaataatattttgtgcatttatgatatgcctctacgtttataatgtat

SEQ ID NO: 8 - DNA, 3169 bp, Glycine max
caaatttcagttatgaataaacaagagatgcattgaaaaggtactcaaagaaaagggtgcaagaacggcattgatagagtaagataaggacggcatttgagtgaaccatcaatttgatccttgagatatgattctatctcattaatagtccttaaagtaagaaaaactacttaaataatcctaaaactattagaaattatactaaataattccttaaatattgaaaaaacctttaaattgatctttgaactttactaaaatacatcaatttaagaactacactaatgggtattcaattcatatcaagtactaggctactgcaaccatagtcctattttttggtgtacgtcttgctcagctgctgtagacagtccagtattgagtttctctgattaaaaataaaatgaaggtgaatttgatgaagtgttttactttttcttttctttttttgaaaaggtgaatttgatgaagtgttttactttgttgcatatcctatacgcaaatggaggattcaacactccaagctgtctaatgcctgtgtaaattacaatagcaaataatgatcttgcatcttggtgctagctaaaagtctatccaaacctacacctactccaagcaatcatcaagtgtagttggtctgtaggtatcggcttgctggccaacatcgtgcatagatagaactggtaggaacattaactgggcgtccaaaggtttgattaggcattacaatactctattgtcattttttatatcatgtcatgcgcatgaattgcatacaatttggctaacataatattgacgtgtccacagtgtctaggattccaaaagccaaaagaaaatatagttaaagctagtgaccggcaggagcagatttttatattaaaccaatgggtattttgttgacagaatgctacatacatatcaacaacggcaattgcttgtgataatagactctccttattctttccctcattacttacatttgttcacaactaaacagcaatggctgtcttcttttcctttcttccgctccgttctcagattctttgtcttgtacttatgttgtttttcactaatattgtagctcaatcacaacagaccaatgaaacaaacttttcatgcccttctgattcaccaccgccttcatgtgaaacctatgtaacatacattgctcagtctccaaattttttgagtctaaccagcatatccaatatatttgacacaagtcctttatccattgcaagagcaagtaacttagagcctgaagacgacaagctgatcgcagaccaagtcttactgataccagtaacctgtggttgcactggaaaccgttctttcgccaatatctcctatgagatcaacccaggtgatagcttctactttgttgcaaccacttcatacgagaatctcacgaattggcgtgtagtgatggatttaaaccccagtctaagtccaaatacgttgccaataggaatccaagtagtatttcctttattctgcaagtgtccttcaaagaaccagttggacaaagggataaagtacctgattacatacgtgtggcagcccagtgacaatgtttcccttgtaagtgaaaagtttggtgcatcaccagaggacatattgagtgaaaacaactatggtcagaactttactgctgcaaacaaccttccagttctgatcccagtgacacgcttgcctgttcttgctcaatctccttcagatgtaagaaaaggcggaattcgtcttccagttataattggtattagcttgggatgcacgctactggtcgtggttttagcagtattactggtgtatgtatactgtctgaaaattaagagtttgaataggagtgcttcatcagctgaaactgcagataaactactttctggagtttcaggctatgtaagtaagcctaccatgtatgaaactgatgcgatcatggaagctaccatgaacctcagtgagcagtgcaagattggggaatcagtgtacaaggcaaacatagagggtaaggttttggcagtaaaaagattcaaggaaaatgtcacagaggagttgaaaattctgcagaaggtgaatcatggaaatctggtgaaattaatgggtgtctcgtcagacaatgatggaaattgttttgtggtttatgaatatgctcaaaatggatctcttgatgagtggctattctacaagtcttgttcagacacatcagactcaagggcctcccttacatggtgtcagaggataagcatagcagtggatgttgcaatgggtttgcagtacatgcatgaacatgcatatccaagaatagtccacagggacatcgcaagcagcaatatccttcttgactcaaacttcaaggccaagatagcaaatttctccatggccagaacttttaccaaccccacgatgccaaagatagatgtctttgcatttggggtggttctgatagagttgcttactggtaggaaagccatgacaaccaaggaaaatggtgaggtagttatgctgtggaaggacatttggaagatctttgatcaagaagagaatagagaggagaggctcaaaaaatggatggatcctaagttagagagttattatcctatagattatgctctcagcttggcctccttggcagtgaattgtactgcagataagtctttgtccagatcaaccattgcagaaattgtccttagcctctcccttctcactcaaccatctcccgtgacattggagagatccttgacttcttctggattagatgtagaagctactcaaattgtcacttccatagcagctcgttgattgagtgagggcaatttagtttctcaaatccatgatggtattttgtttacatgatgattattacatctttagtcattaatggtgggcttggtttaggggagtgtgttcaaaaatttgtttttccatccctgttactttttttaagtttggggtagagtcggcaaaaatgaaagttgcaattgacctcagactaaacttgcttatttcttggtatcttttttgtatgacaattgaaactcaattaaatgatggattatctgttacatgtacaaacaaattcaagcgaaagaataattattcgagtttgaaatatacgtttctaccactggttgcatcaagcttatgtttcatacctcataaagtcaca

SEQ ID NO: 9 - DNA, 1836 bp, Glycine max
atggaactcaaaaaagggttacttgtgttctttttgctgctggagtgtgtttgttacaatgtggaatccaagtgtgtgaagggatgtgatgtagctttcgcttcctactatgtcagtccggatttaagcttagaaaatatagcgcggttgatggaatcaagcattgaagttataatcagcttcaatgaagacaatatatcgaatggttatccgctatccttttacagactcaatattccattcccctgtgactgtattggtggtgagtttctggggcatgtgtttgagtactcagcttctgcaggtgacacctatgattcgattgcgaaagtgacatacgccaatctcaccaccgttgagcttttgcggaggttcaatggctatgatcaaaatggtatacctgcaaatgccagggttaatgtcacggtcaattgttcttgtgggaacagccaggtttcaaaagattatgggatgtttattacctatccactcaggcctgggaataatttgcatgatattgccaatgaggctcgtcttgatgcacagttgctgcagcgttacaatcctggtgtcaatttcagcaaagagagtgggactgttttcattccaggaagagatcaacatggagactatgttcccttgtacccgagaaaaacaggtcttgctaggggtgctgcagttggaatatctatagcaggaatatgcagtcttctattattagtaatttgcttatatggcaagtacttccagaagaaggaaggagagaaaactaaattgccaacagaaaattctatggcgttttcaactcaagatgtctctggaagtgcagaatatgaaacttcaggatccagtgggactgctagtgctactggcctcacaggcattatggtggcaaaatcaatggagttctcatatcaagaactagccaaggctacaaataactttagcttggagaataaaattggtcaaggtggatttggagctgtctattatgcagaactgagaggcgagaaaactgcaattaagaagatggatgtgcaagcatcgacagaatttctttgcgagttgaaggtcttaactcacgttcatcactttaatctggtgcgcttgattggatattgtgttgagggatcccttttccttgtatatgaatatattgacaatggaaacttaggccaatatttgcatggtacagggaaagatcctttgccatggtctggtcgagtgcaaattgcgctagattcagcaagaggccttgaatatattcacgagcacactgtgcctgtgtatatccatcgtgatgtaaaatcagcaaatatattaatagacaagaacatccgtggaaaggttgcagattttggcttgaccaaacttattgaagttggaggctccacacttcacactcgtcttgtgggaacatttggatacatgccaccagaatatgctcaatatggtgacatttctccaaaagtagatgtatatgcttttggagtggttctttatgaacttatttctgcaaagaatgctgttctaaagacaggtgaatctgttgctgaatcaaagggccttgtagctttgtttgaagaagcacttaatcagagtaatccttcagaaagtattcgcaaactggtggatcctaggcttggcgaaaactatccaattgattcagttctcaagattgctcaacttgggagagcttgtacaagagataacccactactacgcccgagtatgaggtctatagttgttgctctcatgacactttcatcacctactgaggattgcgacacttcctacgaaaatcagactctcataaatctactgtctgtgagatga

SEQ ID NO: 10 - DNA, 1857 bp, Glycine max
atggaactcaaaaaatggttactgttctttttgttgctggagtatgtttgttgcaatgcggagtctaagtgtgtgaagggatgtgatgtagctttagcttcatactatgttagtccagggtatttactcttcgaaaatataacgcgcttgatggaatcaattgttctgtccaattctgatgttataatctacaacaaagacaaaatattcaatgaaaatgtgctagcattttccagactcaatattccattcccctgtggctgtatcgatggtgagtttctggggcatgtgtttgagtactcagcttctgcaggtgacacctatgattcgattgcgaaagtgacatatgccaatctcaccactgttgagcttttgcggaggttcaacagttatgatcaaaatggtatacctgcaaatgccacggttaatgtcacggtcaattgttcttgtgggaacagccaggtttcaaaagattatgggctgtttattacctatccactcaggcctgggaataatttgcatgatattgccaacgaggctcgccttgatgcacagttgctacagagttacaatcctagtgtcaatttcagcaaagagagtggggatattgttttcattccaggaagagatcaacatggagattatgttcccttgtaccctagaaaaacaggtcttgctacgagtgcttcagttggaatacctatagcaggaatatgcgttcttctattagtaatttgcatatatgtcaagtacttccagaagaaggaaggagagaaagctaaattggcaacagaaaattctatggcgttttcaactcaagatgtctctggaagtgcagaatatgaaacttcaggatccagtgggactgctagtactagtgctactggccttacaggcattatggtggcaaaatcaatggagttctcatatcaagaactagccaaggctacaaataacttcagcttggagaataaaattggtcaaggtgaatttggaattgtctattatgcagaactgagaggcgagaaaactgcaatcaagaagatggacgtgcaagcatcaacagaatttctttgcgagttgaaggtcttaactcatgttcatcacttgaatctggtgcgcttgattggatattgtgttgaggggtctcttttccttgtatatgaatatattgacaatggaaacttaggccaatatttgcatggtacagggaaagatcctttcctatggtctagccgagtgcaaattgcactagattcagcaagaggccttgaatatattcacgagcacactgtgccagtgtatatccatcgtgatgtaaaatctgcaaatatattaatagacaaaaacttccgtggaaaggttgcagattttggtttgaccaaacttattgaagttggaggttccacacttcaaactcgtcttgtgggaacatttggatacatgccaccagaatatgttcaatatggtgacatttctccaaaagtagatgtatattcttttggagttgttctttatgaacttatttctgcaaagaatgctgtcctaaagacaggagaatctgttgctgaatcaaagggccttgtagctttgtttgaagaagcacttaatcagagtaatccttcagaaagtattcgcaaactggtggatcctaggcttggagaaaactatccaatcgattcagttctcaagattgctcaacttgggagagcttgtacaagagataacccactactacgcccgagtatgaggtctatagttgttgctctcttgacactttcatcacctactgaggattgctatgatgacacttcctacgaaaatcagactctcataaatctactgtctgtgagatga

SEQ ID NO: 11 - DNA, 1797 bp, Glycine max
atggctgtcttctttccctttcttcctctccactctcagattctttgtcttgtgatcatgttgttttccactaatattgtagctcaatcacaacaggacaatagaacaaacttttcatgcccttctgattcaccgccttcatgtgaaacctatgtaacatacattgctcagtctccaaattttttgagtctaaccaacatatccaatatatttgacacaagccctttatccattgcaagagccagtaacttagagcctatggatgacaagctagtcaaagaccaagtcttactcgtaccagtaacctgtggttgcactggaaaccgctcttttgccaatatctcctatgagatcaaccaaggtgatagcttctactttgttgcaaccacttcatacgagaatctcacgaattggcgtgcagtgatggatttaaaccccgttctaagtccaaataagttgccaataggaatccaagtagtatttcctttattctgcaagtgcccttcaaagaaccagttggacaaagagataaagtacctgattacatacgtgtggaagcccggtgacaatgtttcccttgtaagtgacaagtttggtgcatcaccagaggacataatgagtgaaaacaactatggtcagaactttactgctgcaaacaaccttccagttctgatcccagtgacacgcttgccagttcttgctcgatctccttcggacggaagaaaaggcggaattcgtcttccggttataattggtattagcttgggatgcacgctactggttctggttttagcagtgttactggtgtatgtatattgtctgaaaatgaagactttgaataggagtgcttcatcggctgaaactgcagataaactactttctggagtttcaggctatgtaagtaagcctaccatgtatgaaactgatgcgatcatggaagctacaatgaacctcagtgagcagtgcaagattggggaatcagtgtacaaggcaaacatagagggtaaggttttggcagtaaaaagattcaaggaagatgtcacggaagagctgaaaattctgcagaaggtgaatcatgggaatctggtgaaactaatgggtgtctcatcagacaatgatggaaactgttttgtggtttatgaatacgctgaaaatgggtctcttgatgagtggctattctccaagtcttgttcagacacatcaaactcaagggcatcccttacatggtgtcagaggataagcatggcagtggatgttgcgatgggtttgcagtacatgcatgaacatgcttatccaagaatagtccacagggacatcacaagcagtaatatccttcttgactcgaactttaaggccaagatagcaaatttctccatggccagaacttttaccaaccccatgatgccaaagatagatgtctttgcatttggggtggttctgattgagttgcttaccggaaggaaagccatgacaaccaaggaaaatggtgaggtggtcatgctgtggaaggacatttggaagatctttgatcaagaagagaatagagaggagaggctcaaaaaatggatggatcctaagttagagagttattatcctatagattacgctctcagcttggcctccttggcggtgaattgtactgcagataagtctttgtccagaccaaccattgcagaaattgtccttagcctctcccttctcactcaaccatctcccgcaacattggagagatccttgacttcttctggattggatgtagaagctactcaaattgtcacttccatagcagctcgttga

SEQ ID NO: 12 - DNA, 1800 bp, Glycine max
atggctgtcttcttttcctttcttccgctccgttctcagattctttgtcttgtacttatgttgtttttcactaatattgtagctcaatcacaacagaccaatgaaacaaacttttcatgcccttctgattcaccaccgccttcatgtgaaacctatgtaacatacattgctcagtctccaaattttttgagtctaaccagcatatccaatatatttgacacaagtcctttatccattgcaagagcaagtaacttagagcctgaagacgacaagctgatcgcagaccaagtcttactgataccagtaacctgtggttgcactggaaaccgttctttcgccaatatctcctatgagatcaacccaggtgatagcttctactttgttgcaaccacttcatacgagaatctcacgaattggcgtgtagtgatggatttaaaccccagtctaagtccaaatacgttgccaataggaatccaagtagtatttcctttattctgcaagtgtccttcaaagaaccagttggacaaagggataaagtacctgattacatacgtgtggcagcccagtgacaatgtttcccttgtaagtgaaaagtttggtgcatcaccagaggacatattgagtgaaaacaactatggtcagaactttactgctgcaaacaaccttccagttctgatcccagtgacacgcttgcctgttcttgctcaatctccttcagatgtaagaaaaggcggaattcgtcttccagttataattggtattagcttgggatgcacgctactggtcgtggttttagcagtattactggtgtatgtatactgtctgaaaattaagagtttgaataggagtgcttcatcagctgaaactgcagataaactactttctggagtttcaggctatgtaagtaagcctaccatgtatgaaactgatgcgatcatggaagctaccatgaacctcagtgagcagtgcaagattggggaatcagtgtacaaggcaaacatagagggtaaggttttggcagtaaaaagattcaaggaaaatgtcacagaggagttgaaaattctgcagaaggtgaatcatggaaatctggtgaaattaatgggtgtctcgtcagacaatgatggaaattgttttgtggtttatgaatatgctcaaaatggatctcttgatgagtggctattctacaagtcttgttcagacacatcagactcaagggcctcccttacatggtgtcagaggataagcatagcagtggatgttgcaatgggtttgcagtacatgcatgaacatgcatatccaagaatagtccacagggacatcgcaagcagcaatatccttcttgactcaaacttcaaggccaagatagcaaatttctccatggccagaacttttaccaaccccacgatgccaaagatagatgtctttgcatttggggtggttctgatagagttgcttactggtaggaaagccatgacaaccaaggaaaatggtgaggtagttatgctgtggaaggacatttggaagatctttgatcaagaagagaatagagaggagaggctcaaaaaatggatggatcctaagttagagagttattatcctatagattatgctctcagcttggcctccttggcagtgaattgtactgcagataagtctttgtccagatcaaccattgcagaaattgtccttagcctctcccttctcactcaaccatctcccgtgacattggagagatccttgacttcttctggattagatgtagaagctactcaaattgtcacttccatagcagctcgttga

SEQ ID NO: 13 - DNA, 1284 bp, Glycine max
aataaaatattaattatgcttttcaactatatcaatatagtttatagtatctatattaggtgaaatgaagagcattaacgaatcaagagataatataattaattaaataagtatatattcttttaattgatcgtgtttatgaatttattctattttttaaacaattgtatccttcacaagtgccgtgaagcactctttagcatttctagtaaagccaacaataaattatacagagatgtgcgactatgcaatcggtgatatcacacagattcctttttgtttgttattagtgaagtcaatgaagtatattgggtcatagccaagctgcacaggcgtgcctcaaaatttaaaatgcaaaattgttctgtgtttgttagaacaatgagaagacgcgataagaagtggtttgttggcacattggccgacatggttggcatttcggatacaaaggattaaacaaagccagcattctcaatcacaaagattccccttgtcgttctgtatccctctctaccatattcaatgtacaccaaatatgcccttaataaataaaatggcatgcaagttgttacccaagcatgcaataaataaatgacatgcaagtcaactacaataattttctagcctatcctactgtttccttccacactctcattgaaactgtaaatggtataacctatcaggtgttagttctaaaacaggcataaacgtgtgcatatgaattcatatactctaacacaaatttcggacaccactaatatctaaaatataggtatttgggtactacttacactcacaaataaagagattctaatcaaatgaaaaattaataacatataataaatcaaatatctaaattgatgttatattcatctatttaaaccagttttaatttttatgtttttcaagtgtattaattgtgtaaaggtgacgccttaagtgtttaagtcaataaagagtaatttttgaaccagacacctaataagaagtgttaaacaagtgtccaggtgtatcggtgttgaaaacatatatgaaacaacgacacttcaaacaagcaaggcctccgtgtttcataggtttaatgttcgcacgcattcacttaagttacctacaacattcttttatgttttagtgattaaaagaggaagtgtgacattggtttcaactttgaagagaaaaagaaatgaaaataattattgattaaaccctgtatagaaagtcctagaaatcttgttttctgatttggatttctttgtgttcctcttatttgctccctgtgatcca

SEQ ID NO: 14 - DNA, 967 bp, Glycine max
aaaactggttcataagggggtggtctacccaactatataagcacttatcatattcatgaattactcgatgtgagactattcttaacatttgttatgtcaacggagtatatttggtcatagccaagctgcacaggcgtgcctcaaaatttaaaatggaaaattgttcttcgtttgttatgttagaacaatgagaggacacaatacgaagtggtttgttggcacattggccgacacggttggcatttcggatagaaaggatcaaacaaagccaacattttcaatcacagagatttccgcgtccatattatgcagccctctctaccataaaaaatatcactatattcaaagtacaccaaatatgtcctcctcaataaatgacatgcaagttgttatccaaaattaaataaataaataaattagggttcttgctaatagggtattggttaaggaattaaaacgagaaaatatttaatgtaaaaaccataagagaacataaaaaagtcaagtaaaacataattttgtgcatttgaataaatttttttttcttttagtttcttaatcaatatcttaagaacaccgatcaatatttgtcatataaataaatgacatgcaagtcaacttcaataattttctagccaatcctactgtttccttccacattctcgtggaaaactatttagcgttataacctatcaggtgtatgttctgaaaaaactaaaaagcataaacgtgtgcatgtgaattcttagtttatgttcattcacttaattagttacacctttatacttttattttatgttttgagttacttttctatagtctgtgtgttaattaaaagaggaagtgtgacattggtttcaagagaaaaaagaaatgaatatgattaaggctggtgtataaagtcctagaaatccacttttctgatttgagtttctttatgtctctcttgtgtgctctccgtgaccca

SEQ ID NO: 15 - DNA, 870 bp, Glycine max
tcaggtactcaaagaaaagggtgcgagaacgacattgagagagtaacataaggacggcattcaagggaaccatcaatctgatccttgagatatgattctctctcattaatagtccttaaagtaagaaaaactacttatatagttctaaaagttttagaaattataccaaataatttcttaaatattgaaaaaccctttaaattgatctttgaactttactaaaataaatcatcaatttaaggactaaactaatgggtattcaattcatatcaagtactaggctactacaaccataatcctattctttgatgtacgtcttgctcagctgctgaagacagtccagtattgagtttctttgattaaagataaaatgaaggtgaatttgatgaagtgctttagtatgttgaatcctatgcaaatggacaattcaacactccaagctgtgtaaattacaatagcaaataatggtctctgtccttgttaaaaattatcgagtttagttggtctgtaggtgtcggcttgctggccaacatcgtgcatagataaaacattaactggccgtccaaaggttggattaggcattgcaatactcttattgtcatttttttttatagcatgcgcatgaattgcatacaattaggctaatataatattgacgtgtccacagtgtcaaagattcggaaaaccaaaagaaaatatagttaaagctaatgacaaagacatgagcagatttttatatatattaaaccaaaggctatttttttgttgacaaagaatgcttcatacatatcaacaactgcagttgcctgtgataatagactctccttattctttccctcgttacttacatttgttcacaactaaacagca

SEQ ID NO: 16 - DNA, 1002 bp, Glycine max
caaatttcagttatgaataaacaagagatgcattgaaaaggtactcaaagaaaagggtgcaagaacggcattgatagagtaagataaggacggcatttgagtgaaccatcaatttgatccttgagatatgattctatctcattaatagtccttaaagtaagaaaaactacttaaataatcctaaaactattagaaattatactaaataattccttaaatattgaaaaaacctttaaattgatctttgaactttactaaaatacatcaatttaagaactacactaatgggtattcaattcatatcaagtactaggctactgcaaccatagtcctattttttggtgtacgtcttgctcagctgctgtagacagtccagtattgagtttctctgattaaaaataaaatgaaggtgaatttgatgaagtgttttactttttcttttctttttttgaaaaggtgaatttgatgaagtgttttactttgttgcatatcctatacgcaaatggaggattcaacactccaagctgtctaatgcctgtgtaaattacaatagcaaataatgatcttgcatcttggtgctagctaaaagtctatccaaacctacacctactccaagcaatcatcaagtgtagttggtctgtaggtatcggcttgctggccaacatcgtgcatagatagaactggtaggaacattaactgggcgtccaaaggtttgattaggcattacaatactctattgtcattttttatatcatgtcatgcgcatgaattgcatacaatttggctaacataatattgacgtgtccacagtgtctaggattccaaaagccaaaagaaaatatagttaaagctagtgaccggcaggagcagatttttatattaaaccaatgggtattttgttgacagaatgctacatacatatcaacaacggcaattgcttgtgataatagactctccttattctttccctcattacttacatttgttcacaactaaacagca

SEQ ID NO: 17 - DNA, 1860 bp, Glycine max
atggaactcaaaaaatggttactgttctttttgttgctggagtatgtttgttgcaatgcggagtctaagtgtgtgaagggatgtgatgtagctttagcttcatactatgttagtccagggtatttactcttcgaaaatataacgcgcttgatggaatcaattgttctgtccaattctgatgttataatctacaacaaagacaaaatattcaatgaaaatgtgctagcattttccagactcaatattccattcccctgtggctgtatcgatggtgagtttctggggcatgtgtttgagtactcagcttctgcaggtgacacctatgattcgattgcgaaagtgacatatgccaatctcaccactgttgagcttttgcggaggttcaacagttatgatcaaaatggtatacctgcaaatgccacggttaatgtcacggtcaattgttcttgtgggaacagccaggtttcaaaagattatgggctgtttattacctatccactcaggcctgggaataatttgcatgatattgccaacgaggctcgccttgatgcacagttgctacagagttacaatcctagtgtcaatttcagcaaagagagtggggatattgttttcattccaggaagagatcaacatggagattatgttcccttgtaccctagaaaaacagcaggtcttgctacgagtgcttcagttggaatacctatagcaggaatatgcgttcttctattagtaatttgcatatatgtcaagtacttccagaagaaggaaggagagaaagctaaattggcaacagaaaattctatggcgttttcaactcaagatgtctctggaagtgcagaatatgaaacttcaggatccagtgggactgctagtactagtgctactggccttacaggcattatggtggcaaaatcaatggagttctcatatcaagaactagccaaggctacaaataacttcagcttggagaataaaattggtcaaggtgaatttggaattgtctattatgcagaactgagaggcgagaaaactgcaatcaagaagatggacgtgcaagcatcaacagaatttctttgcgagttgaaggtcttaactcatgttcatcacttgaatctggtgcgcttgattggatattgtgttgaggggtctcttttccttgtatatgaatatattgacaatggaaacttaggccaatatttgcatggtacagggaaagatcctttcctatggtctagccgagtgcaaattgcactagattcagcaagaggccttgaatatattcacgagcacactgtgccagtgtatatccatcgtgatgtaaaatctgcaaatatattaatagacaaaaacttccgtggaaaggttgcagattttggtttgaccaaacttattgaagttggaggttccacacttcaaactcgtcttgtgggaacatttggatacatgccaccagaatatgttcaatatggtgacatttctccaaaagtagatgtatattcttttggagttgttctttatgaacttatttctgcaaagaatgctgtcctaaagacaggagaatctgttgctgaatcaaagggccttgtagctttgtttgaagaagcacttaatcagagtaatccttcagaaagtattcgcaaactggtggatcctaggcttggagaaaactatccaatcgattcagttctcaagattgctcaacttgggagagcttgtacaagagataacccactactacgcccgagtatgaggtctatagttgttgctctcttgacactttcatcacctactgaggattgctatgatgacacttcctacgaaaatcagactctcataaatctactgtctgtgagatga

SEQ ID NO: 18 - DNA, 1654 bp, Glycine max
atggaactcaaaaaatggttactgttctttttgttgctggagtatgtttgttgcaatgcggagtctaagtgtgtgaagggatgtgatgtagctttagcttcatactatgttagtccagggtatttactcttcgaaaatataacgcgcttgatggaatcaattgttctgtccaattctgatgttataatctacaacaaagacaaaatattcaatgaaaatgtgctagcattttccagactcaatattccattcccctgtggctgtatcgatggtgagtttctggggcatgtgtttgagtactcagcttctgcaggtgacacctatgattcgattgcgaaagtgacatatgccaatctcaccactgttgagcttttgcggaggttcaacagttatgatcaaaatggtatacctgcaaatgccacggttaatgtcacggtcaattgttcttgtgggaacagccaggtttcaaaagattatgggctgtttattacctatccactcaggcctgggaataatttgcatgatattgccaacgaggctcgccttgatgcacagttgctacagagttacaatcctagtgtcaatttcagcaaagagagtggggatattgttttcattccaggaagagatcaacatggagattatgttcccttgtaccctagaaaaacaggtcttgctacgagtgcttcagttggaatacctatagcaggaatatgcgttcttctattagtaatttgcatatatgtcaagtacttccagaagaaggaaggagagaaagctaaattggcaacagaaaattctatggcgttttcaactcaagatgaaaactgcaatcaagaagatggacgtgcaagcatcaacagaatttctttgcgagttgaaggtcttaactcatgttcatcacttgaatctggtgcgcttgattggatattgtgttgaggggtctcttttccttgtatatgaatatattgacaatggaaacttaggccaatatttgcatggtacagggaaagatcctttcctatggtctagccgagtgcaaattgcactagattcagcaagaggccttgaatatattcacgagcacactgtgccagtgtatatccatcgtgatgtaaaatctgcaaatatattaatagacaaaaacttccgtggaaaggttgcagattttggtttgaccaaacttattgaagttggaggttccacacttcaaactcgtcttgtgggaacatttggatacatgccaccagaatatgttcaatatggtgacatttctccaaaagtagatgtatattcttttggagttgttctttatgaacttatttctgcaaagaatgctgtcctaaagacaggagaatctgttgctgaatcaaagggccttgtagctttgtttgaagaagcacttaatcagagtaatccttcagaaagtattcgcaaactggtggatcctaggcttggagaaaactatccaatcgattcagttctcaagattgctcaacttgggagagcttgtacaagagataacccactactacgcccgagtatgaggtctatagttgttgctctcttgacactttcatcacctactgaggattgctatgatgacacttcctacgaaaatcagactctcataaatctactgtctgtgagatga

SEQ ID NO: 19 - DNA, 1708 bp, Glycine max
atggaactcaaaaaatggttactgttctttttgttgctggagtatgtttgttgcaatgcggagtctaagtgtgtgaagggatgtgatgtagctttagcttcatactatgttagtccagggtatttactcttcgaaaatataacgcgcttgatggaatcaattgttctgtccaattctgatgttataatctacaacaaagacaaaatattcaatgaaaatgtgctagcattttccagactcaatattccattcccctgtggctgtatcgatggtgagtttctggggcatgtgtttgagtactcagcttctgcaggtgacacctatgattcgattgcgaaagtgacatatgccaatctcaccactgttgagcttttgcggaggttcaacagttatgatcaaaatggtatacctgcaaatgccacggttaatgtcacggtcaattgttcttgtgggaacagccaggtttcaaaagattatgggctgtttattacctatccactcaggcctgggaataatttgcatgatattgccaacgaggctcgccttgatgcacagttgctacagagttacaatcctagtgtcaatttcagcaaagagagtggggatattgttttcattccaggaagagatcaacatggagattatgttcccttgtaccctagaaaaacagcaggtcttgctacgagtgcttcagttggaatacctatagcaggaatatgcgttcttctattagtaatttgcatatatgtcaagtacttccagaagaaggaaggagagaaagctaaattggcaacagaaaattctatggcgttttcaactcaagatgtctctggaagtgcagaatatgaaacttcaggatccagtgggactgctagtactagtgctactggccttacaggcattatggtggcaaaatcaatggagttctcatatcaagaactagccaaggctacaaataacttcagcttggagaataaaattggtcaaggtgaatttggaattgtctattatgcagaactgagaggcgagaaaactgcaatcaagaagatggacgtgcaagcatcaacagaatttctttgcgagttgaaggtcttaactcatgttcatcacttgaatctggtgcgcttgattggatattgtgttgaggggtctcttttccttgtatatgaatatattgacaatggaaacttaggccaatatttgcatggtacaggttgcagattttggtttgaccaaacttattgaagttggaggttccacacttcaaactcgtcttgtgggaacatttggatacatgccaccagaatatgttcaatatggtgacatttctccaaaagtagatgtatattcttttggagttgttctttatgaacttatttctgcaaagaatgctgtcctaaagacaggagaatctgttgctgaatcaaagggccttgtagctttgtttgaagaagcacttaatcagagtaatccttcagaaagtattcgcaaactggtggatcctaggcttggagaaaactatccaatcgattcagttctcaagattgctcaacttgggagagcttgtacaagagataacccactactacgcccgagtatgaggtctatagttgttgctctcttgacactttcatcacctactgaggattgctatgatgacacttcctacgaaaatcagactctcataaatctactgtctgtgagatga

SEQ ID NO: 20 - DNA, 20 bp, Artificial Sequence
  Synthetic primer
gctctccttttcgcatcatc

SEQ ID NO: 21 - DNA, 20 bp, Artificial Sequence
  Synthetic primer
ccaagttgagcaatctgcaa

SEQ ID NO: 22 - DNA, 20 bp, Artificial Sequence
  Synthetic primer
atgcttggggttgtttgaag

SEQ ID NO: 23 - DNA, 20 bp, Artificial Sequence
  Synthetic primer
caacgtgcttccaaaagtca

SEQ ID NO: 24 - DNA, 20 bp, Artificial Sequence
  Synthetic primer
cagaaacttgccaatccacc

SEQ ID NO: 25 - DNA, 20 bp, Artificial Sequence
  Synthetic primer
ccaagttgagcaatctgcaa

SEQ ID NO: 26 - DNA, 20 bp, Artificial Sequence
  Synthetic primer
gccttgatgcacagttgcta

SEQ ID NO: 27 - DNA, 20 bp, Artificial Sequence
  Synthetic primer
cgtgcaagcatcaacagaat

SEQ ID NO: 28 - DNA, 21 bp, Artificial Sequence
  Synthetic primer
attcacgagcacactgtgcct

SEQ ID NO: 29 - DNA, 21 bp, Artificial Sequence
  Synthetic primer
gccaaaatctgcaacctttcc

SEQ ID NO: 30 - DNA, 21 bp, Artificial Sequence
  Synthetic primer
attcacgagcacactgtgcca

SEQ ID NO: 31 - DNA, 21 bp, Artificial Sequence
  Synthetic primer
accaaaatctgcaacctttcc

SEQ ID NO: 32 - DNA, 24 bp, Artificial Sequence
  Synthetic primer
ggtcgcacaactggtattgtattg

SEQ ID NO: 33 - DNA, 20 bp, Artificial Sequence
  Synthetic primer
ctcagcagaggtggtgaaca

SEQ ID NO: 34 - DNA, 20 bp, Artificial Sequence
  Synthetic primer
aacacatgccccagaaactc

SEQ ID NO: 35 - DNA, 20 bp, Artificial Sequence
  Synthetic primer
tcaggcctgggaataatttg

SEQ ID NO: 36 - DNA, 20 bp, Artificial sequence
  Synthetic primer
ttgaaccctcaatacgctga

SEQ ID NO: 37 - DNA, 21 bp, Artificial sequence
  Synthetic primer
ctttcagaaaaacaggtttgg

SEQ ID NO: 38 - DNA, 20 bp, Artificial sequence
  Synthetic primer
tccgggtaaagtctctggaa

SEQ ID NO: 39 - DNA, 20 bp, Artificial sequence
  Synthetic primer
tgtgcaagcatcgacagaat

SEQ ID NO: 40 - DNA, 20 bp, Artificial sequence
  Synthetic primer
ttggcataagcagttcgatg

SEQ ID NO: 41 - DNA, 20 bp, Artificial sequence
  Synthetic primer
attcagcaagaggccttgaa

SEQ ID NO: 42 - DNA, 20 bp, Artificial sequence
  Synthetic primer
tgaacggatcataacgacga

SEQ ID NO: 43 - DNA, 20 bp, Artificial sequence
  Synthetic primer
ccaagttgagcaatctgcaa

SEQ ID NO: 44 - DNA, 20 bp, Artificial sequence
  Synthetic primer
gctcaacttgggagagcttg

SEQ ID NO: 45 - DNA, 20 bp, Artificial sequence
  Synthetic primer
gagtttctggggcatgtgtt

SEQ ID NO: 46 - DNA, 20 bp, Artificial sequence
  Synthetic primer
tcaggcctgggaataatttg

SEQ ID NO: 47 - DNA, 22 bp, Artificial sequence
  Synthetic primer
acatgatgtgaaaaggagagca

SEQ ID NO: 48 - DNA, 21 bp, Artificial sequence
  Synthetic primer
cttgcagaaaaacaggtttgg

SEQ ID NO: 49 - DNA, 20 bp, Artificial sequence
  Synthetic primer
tccgggtaaagtctctggaa

SEQ ID NO: 50 - DNA, 20 bp, Artificial sequence
  Synthetic primer
cgtgcaagcatcaacagaat

SEQ ID NO: 51 - DNA, 20 bp, Artificial sequence
  Synthetic primer
attcagcaagaggccttgaa

SEQ ID NO: 52 - DNA, 20 bp, Artificial sequence
  Synthetic primer
ttgattgtggaaaacgagca

SEQ ID NO: 53 - DNA, 20 bp, Artificial sequence
  Synthetic primer
ccaagttgagcaatctgcaa

SEQ ID NO: 54 - DNA, 20 bp, Artificial sequence
  Synthetic primer
gctcaacttgggagagcttg

SEQ ID NO: 55 - DNA, 22 bp, Artificial sequence
  Synthetic primer
attgcaagagccagtaacatag

SEQ ID NO: 56 - DNA, 21 bp, Artificial sequence
  Synthetic primer
gtatgttcatgcatgtattgc

SEQ ID NO: 57 - DNA, 19 bp, Artificial sequence
  Synthetic primer
gatgttggccagcaagccg

SEQ ID NO: 58 - DNA, 21 bp, Artificial sequence
  Synthetic primer
aagttgcaattgacctcagac

SEQ ID NO: 59 - DNA, 22 bp, Artificial sequence
  Synthetic primer
taggtttcacatgaaggcggtg

SEQ ID NO: 60 - DNA, 32 bp, Artificial sequence
  Synthetic primer
ggggatccaccattgctgtttagttgtgaaca

SEQ ID NO: 61 - DNA, 24 bp, Artificial sequence
  Synthetic primer
ggaagcttggtttaggggagtgtg

SEQ ID NO: 62 - DNA, 24 bp, Artificial sequence
  Synthetic primer
gtcacttccatagcagctcgttga

SEQ ID NO: 63 - DNA, 22 bp, Artificial sequence
  Synthetic primer
gtaagggaggcccttgagtctg

SEQ ID NO: 64 - DNA, 22 bp, Artificial sequence
  Synthetic primer
acctgtggttgcactggaaacc

SEQ ID NO: 65 - DNA, 20 bp, Artificial sequence
  Synthetic primer
gtatgcaattcatgcgcatg

SEQ ID NO: 66 - DNA, 30 bp, Artificial sequence
  Synthetic primer
ggggagctcatatcaacaactgcagttgcc

SEQ ID NO: 67 - DNA, 26 bp, Artificial sequence
  Synthetic primer
ggtatgaaacataagcttaatgcaat

SEQ ID NO: 68 - DNA, 30 bp, Artificial sequence
  Synthetic primer
ggggagctcatatcaacaacggcaattgct

SEQ ID NO: 69 - DNA, 24 bp, Artificial sequence
  Synthetic primer
cataagcttgatgcaaccagtggt

SEQ ID NO: 70 - DNA, 27 bp, Artificial sequence
  Synthetic primer
aaaggtacccaaagaaaagggtgcaag

SEQ ID NO: 71 - DNA, 21 bp, Artificial sequence
  Synthetic primer
cactcaaatgccgtccttatc

SEQ ID NO: 72 - DNA, 20 bp, Artificial sequence
  Synthetic primer
tctgcagaaggtgaatcatg

SEQ ID NO: 73 - DNA, 22 bp, Artificial sequence
  Synthetic primer
ttcatgcatgtactgcaaaccc

SEQ ID NO: 74 - DNA, 20 bp, Artificial sequence
  Synthetic primer
gccaaggaggccaagctgag

SEQ ID NO: 75 - DNA, 19 bp, Artificial sequence
  Synthetic primer
gcatttggggtggttctga

SEQ ID NO: 76 - DNA, 728 bp, Glycine max
atccttcacaagtgccgtgaagcactctttagcatttctagtaaagccaacaataaattatacagagatgtgcgactatgcaatcggtgatatcacacagattcctttttgtttgttattagtgaagtcaatgaagtatattgggtcatagccaagctgcacaggcgtgcctcaaaatttaaaatgcaaaattgttctgtgtttgttagaacaatgagaagacgcgataagaagtggtttgttggcacattggccgacatggttggcatttcggatacaaaggattaaacaaagccagcattctcaatcacaaagattccccttgtcgttctgtatccctctctaccatattcaatgtacaccaaatatgcccttaataaataaaatggcatgcaagttgttacccaagcatgcaataaataaatgacatgcaagtcaactacaataattttctagcctatcctactgtttccttccacactctcattgaaactgtaaatggtataacctatcaggtgttagttctaaaacaggcataaacgtgtgcatatgaattcatatactctaacacaaatttcggacaccactaatatctaaaatataggtatttgggtactacttacactcacaaataaagagattctaatcaaatgaaaaattaataacatataataaatcaaatatctaaattgatgttatattcatctatttaaaccagttttaatttt

SEQ ID NO: 77 - DNA, 649 bp, Glycine max
aaaactggttcataagggggtggtctacccaactatataagcacttatcatattcatgaattactcgatgtgagactattcttaacatttgttatgtcaacggagtatatttggtcatagccaagctgcacaggcgtgcctcaaaatttaaaatggaaaattgttcttcgtttgttatgttagaacaatgagaggacacaatacgaagtggtttgttggcacattggccgacacggttggcatttcggatagaaaggatcaaacaaagccaacattttcaatcacagagatttccgcgtccatattatgcagccctctctaccataaaaaatatcactatattcaaagtacaccaaatatgtcctcctcaataaatgacatgcaagttgttatccaaaattaaataaataaataaattagggttcttgctaatagggtattggttaaggaattaaaacgagaaaatatttaatgtaaaaaccataagagaacataaaaaagtcaagtaaaacataattttgtgcatttgaataaatttttttttcttttagtttcttaatcaatatcttaagaacaccgatcaatatttgtcatataaataaatgacatgcaagtcaacttcaataattttctagccaatcctactgtt

SEQ ID NO: 78 - DNA, 712 bp, Lotus japonicus
taataagtcattgttgtgggcgaataccctaaaataagaataaaattaaatatagcatccaagttattgcccaaatatataaacaatggtattgttgacattattaggcataaaagcagtaggtaagtgtattatatttatttaattttttaaaattttgaaattaattaataattgttaacataagtaaaccatttttagcaaaaactctacacttctattaccttaacaagtacatttttgatggtacaccttaacaattaacaagtcatatgattgacaaacatattttatatgctttacaatttattctaaaatcaaagtttatgggaagaagctcataaaagtagttcctgggtgttttttagaatagagaagttgatcatgttagaaattaagttaaaaatgagttgaaagtgatttatgtttgattatatttatgagaaaaatgaattgtctgatgtaatattgtaaaatctaacaattaattaagtaccacagaaactagaatttatagcttcaccttagaattgattttggagttaaaatcaattattaaaggagcaattattaaaggagacatccaaatacactagttaattttgacaatcaattctaacacttgcaaatgtgtaaccaaacttactatcagtaagtgaactaatgattcccaagtcaacttttgttctagctagccaaccgttactatgtt

SEQ ID NO: 79 - protein, 621 aa, Lotus japonicus
MKLKTGLLLFFILLLGHVCFHVESNCLKGCDLALASYYILPGVFILQNITTFMQSEIVSSNDAITSYNKDKILNDINIQSFQRLNIPFPCDCIGGEFLGHVFEYSASKGDTYETIANLYYANLTTVDLLKRFNSYDPKNIPVNAKVNVTVNCSCGNSQVSKDYGLFITYPIRPGDTLQDIANQSSLDAGLIQSFNPSVNFSKDSGIAFIPGRYKNGVYVPLYHRTAGLASGAAVGISIAGTFVLLLLAFCMYVRYQKKEEEKAKLPTDISMALSTQDASSSAEYETSGSSGPGTASATGLTSIMVAKSMEFSYQELAKATNNFSLDNKIGQGGFGAVYYAELRGKKTAIKKMDVQASTEFLCELKVLTHVHHLNLVRLIGYCVEGSLFLVYEHIDNGNLGQYLHGSGKEPLPWSSRVQIALDAARGLEYIHEHTVPVYIHRDVKSANILIDKNLRGKVADFGLTKLIEVGNSTLQTRLVGTFGYMPPEYAQYGDISPKIDVYAFGVVLFELISAKNAVLKTGELVAESKGLVALFEEALNKSDPCDALRKLVDPRLGENYPIDSVLKIAQLGRACTRDNPLLRPSMRSLVVALMTLSSLTEDCDDESSYESQTLINLLSVR

SEQ ID NO: 80 - protein, 620 aa, Medicago truncatula
MNLKNGLLLFILFLDCVFFKVESKCVKGCDVALASYYIIPSIQLRNISNFMQSKIVLTNSFDVIMSYNRDVVFDKSGLISYTRINVPFPCECIGGEFLGHVFEYTTKEGDDYDLIANTYYASLTTVELLKKFNSYDPNHIPVKAKINVTVICSCGNSQISKDYGLFVTYPLRSDDTLAKIATKAGLDEGLIQNFNQDANFSIGSGIVFIPGRDQNGHFFPLYSRTGIAKGSAVGIAMAGIFGLLLFVIYIYAKYFQKKEEEKTKLPQTSRAFSTQDASGSAEYETSGSSGHATGSAAGLTGIMVAKSTEFTYQELAKATNNFSLDNKIGQGGFGAVYYAELRGEKTAIKKMDVQASSEFLCELKVLTHVHHLNLVRLIGYCVEGSLFLVYEHIDNGNLGQYLHGIGTEPLPWSSRVQIALDSARGLEYIHEHTVPVYIHRDVKSANILIDKNLRGKVADFGLTKLIEVGNSTLHTRLVGTFGYMPPEYAQYGDVSPKIDVYAFGVVLYELITAKNAVLKTGESVAESKGLVQLFEEALHRMDPLEGLRKLVDPRLKENYPIDSVLKMAQLGRACTRDNPLLRPSMRSIVVALMTLSSPTEDCDDDSSYENQSLINLLSTR

SEQ ID NO: 81 - protein, 595 aa, Lotus japonicus
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVYVYCRRKKALNRTASSAETADKLLSGVSGYVSKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 82 - protein, 594 aa, Pisum sativum
MAIFFLPSSSHALFLALMFFVTNISAQPLQLSGTNFSCPVDSPPSCETYVTYFARSPNFLSLTNISDIFDMSPLSIAKASNIEDEDKKLVEGQVLLIPVTCGCTRNRYFANFTYTIKLGDNYFIVSTTSYQNLTNYVEMENFNPNLSPNLLPPEIKVVVPLFCKCPSKNQLSKGIKHLITYVWQANDNVTRVSSKFGASQVDMFTENNQNFTASTNVPILIPVTKLPVIDQPSSNGRKNSTQKPAFIIGISLGCAFFVVVLTLSLVYVYCLKMKRLNRSTSLAETADKLLSGVSGYVSKPTMYEMDAIMEATMNLSENCKIGESVYKANIDGRVLAVKKIKKDASEELKILQKVNHGNLVKLMGVSSDNEGNCFLVYEYAENGSLDEWLFSELSKTSNSVVSLTWSQRITVAVDVAVGLQYMHEHTYPRIIHRDITTSNILLDSNFKAKIANFSMARTSTNSMMPKIDVFAFGVVLIELLTGKKAITTMENGEVVILWKDFWKIFDLEGNREESLRKWMDPKLENFYPIDNALSLASLAVNCTADKSLSRPSIAEIVLCLSLLNQSSSEPMLERSLTSGLDVEATHVVTSIVAR

SEQ ID NO: 83 - protein, 595 aa, Medicago truncatula
MSAFFLPSSSHALFLVLMLFFLTNISAQPLYISETNFTCPVDSPPSCETYVAYRAQSPNFLSLSNISDIFNLSPLRIAKASNIEAEDKKLIPDQLLLVPVTCGCTKNHSFANITYSIKQGDNFFILSITSYQNLTNYLEFKNFNPNLSPTLLPLDTKVSVPLFCKCPSKNQLNKGIKYLITYVWQDNDNVTLVSSKFGASQVEMLAENNHNFTASTNRSVLIPVTSLPKLDQPSSNGRKSSSQNLALIIGISLGSAFFILVLTLSLVYVYCLKMKRLNRSTSSSETADKLLSGVSGYVSKPTMYEIDAIMEGTTNLSDNCKIGESVYKANIDGRVLAVKKIKKDASEELKILQKVNHGNLVKLMGVSSDNDGNCFLVYEYAENGSLEEWLFSESSKTSNSVVSLTWSQRITIAMDVAIGLQYMHEHTYPRIIHRDITTSNILLGSNFKAKIANFGMARTSTNSMMPKIDVFAFGVVLIELLTGKKAMTTKENGEVVILWKDFWKIFDLEGNREERLRKWMDPKLESFYPIDNALSLASLAVNCTADKSLSRPTIAEIVLCLSLLNQPSSEPMLERSLTSGLDAEATHVVTSVIAR

SEQ ID NO: 84 - DNA, 1866 bp, Lotus japonicus
atgaagctaaaaactggtctacttttgtttttcattcttttgctggggcatgtttgtttccatgtggaatcaaactgtctgaaggggtgtgatctagctttagcttcctattatatcttgcctggtgttttcatcttacaaaacataacaacctttatgcaatcagagattgtctcaagtaatgatgccataaccagctacaacaaagacaaaattctcaatgatatcaacatccaatcctttcaaagactcaacattccatttccatgtgactgtattggtggtgagtttctagggcatgtatttgagtactcagcttcaaaaggagacacttatgaaactattgccaacctctactatgcaaatttgacaacagttgatcttttgaaaaggttcaacagctatgatccaaaaaacatacctgttaatgccaaggttaatgtcactgttaattgttcttgtgggaacagccaggtttcaaaagattatggcttgtttattacctatcccattaggcctggggatacactgcaggatattgcaaaccagagtagtcttgatgcagggttgatacagagtttcaacccaagtgtcaatttcagcaaagatagtgggatagctttcattcctggaagatataaaaatggagtctatgttcccttgtaccacagaaccgcaggtctagctagtggtgcagctgttggtatatctattgcaggaaccttcgtgcttctgttactagcattttgtatgtatgttagataccagaagaaggaagaagagaaagctaaattgccaacagatatttctatggccctttcaacacaagatgcctctagtagtgcagaatatgaaacttctggatccagtgggccagggactgctagtgctacaggtcttactagcattatggtggcgaaatcaatggagttctcatatcaggaactagcgaaggctacaaataactttagcttggataataaaattggtcaaggtggatttggagctgtctattatgcagaattgagaggcaagaaaacagcaattaagaagatggatgtacaagcatcaacagaatttctttgtgagttgaaggtcttaacacatgttcaccacttgaatctggtgcgcttgattggatactgcgttgagggatctctattccttgtttatgaacatattgacaatggaaacttaggccaatatttgcatggttcaggtaaagaaccattgccatggtctagccgagtacaaatagctctagatgcagcaagaggccttgaatacattcatgagcacactgtgcctgtgtatatccatcgcgatgtgaaatctgcaaacatattgatagataagaacttgcgtggaaaggttgcagattttggcttgaccaagcttattgaagttgggaactccacactacaaactcgtctggtgggaacatttggatacatgcccccagaatatgctcaatatggtgatatttctccaaaaatagatgtatatgcatttggagttgttctttttgaacttatttctgcaaagaatgctgttctgaagacaggtgaattagttgctgaatcaaagggccttgtagctttgtttgaagaagcacttaataagagtgatccttgtgatgctcttcgcaaactggtggatcctaggcttggagaaaactatccaattgattctgttctcaagattgcacaactagggagagcttgtacaagagataatccactgctaagaccaagtatgagatctttagttgttgctcttatgaccctttcatcacttactgaggattgtgatgatgaatcttcctacgaaagtcaaactctcataaatttactgtctgtgagataa

SEQ ID NO: 85 - DNA, 1863 bp, Medicago trunculata
atgaatctcaaaaatggattactattgttcattctgtttctggattgtgtttttttcaaagttgaatccaaatgtgtaaaagggtgtgatgtagctttagcttcctactatattataccatcaattcaactcagaaatatatcaaactttatgcaatcaaagattgttcttaccaattcctttgatgttataatgagctacaatagagacgtagtattcgataaatctggtcttatttcctatactagaatcaacgttccgttcccatgtgaatgtattggaggtgaatttctaggacatgtgtttgaatatacaacaaaagaaggagacgattatgatttaattgcaaatacttattacgcaagtttgacaactgttgagttattgaaaaagttcaacagctatgatccaaatcatatacctgttaaggctaagattaatgtcactgtaatttgttcatgtgggaatagccagatttcaaaagattatggcttgtttgttacctatccactcaggtctgatgatactcttgcgaaaattgcgaccaaagctggtcttgatgaagggttgatacaaaatttcaatcaagatgccaatttcagcataggaagtgggatagtgttcattccaggaagagatcaaaatggacatttttttcctttgtattctagaacaggtattgctaagggttcagctgttggtatagctatggcaggaatatttggacttctattatttgttatctatatatatgccaaatacttccaaaagaaggaagaagagaaaactaaacttccacaaacttctagggcattttcaactcaagatgcctcaggtagtgcagaatatgaaacttcaggatccagtgggcatgctactggtagtgctgccggccttacaggcattatggtggcaaagtcgacagagtttacgtatcaagaattagccaaggcgacaaataatttcagcttggataataaaattggtcaaggtggatttggagctgtctattatgcagaacttagaggcgagaaaacagcaattaagaagatggatgtacaagcatcgtccgaatttctctgtgagttgaaggtcttaacacatgttcatcacttgaatctggtgcggttgattggatattgcgttgaagggtcacttttcctcgtatatgaacatattgacaatggaaacttgggtcaatatttacatggtataggtacagaaccattaccatggtctagtagagtgcagattgctctagattcagccagaggcctagaatacattcatgaacacactgtgcctgtttatatccatcgcgacgtaaaatcagcaaatatattgatagacaaaaatttgcgtggaaaggttgctgattttggcttgaccaaacttattgaagttggaaactcgacacttcacactcgtcttgtgggaacatttggatacatgccaccagaatatgctcaatatggcgatgtttctccaaaaatagatgtatatgcttttggcgttgttctttatgaacttattactgcaaagaatgctgtcctgaagacaggtgaatctgttgcagaatcaaagggtcttgtacaattgtttgaagaagcacttcatcgaatggatcctttagaaggtcttcgaaaattggtggatcctaggcttaaagaaaactatcccattgattctgttctcaagatggctcaacttgggagagcatgtacgagagacaatccgctactacgcccaagcatgagatctatagttgttgctcttatgacactttcatcaccaactgaagattgtgatgatgactcttcatatgaaaatcaatctctcataaatctgttgtcaactagatga

SEQ ID NO: 86 - DNA, 21 bp, Artificial Sequence
  Synthetic primer
ttttttttttttttttttttv